(19) United States
(12) Patent Application Publication
(10) Pub. No.: US 2021/0115087 A1
(43) Pub. Date: Apr. 22, 2021

(54) CYCLIC PEPTIDE INHIBITORS OF HEDGEHOG PROTEINS

(71) Applicant: University of Rochester, Rochester, NY (US)

(72) Inventors: Rudi Fasan, Rochester, NY (US); Andrew Owens, Rochester, NY (US)

(21) Appl. No.: 17/136,285

(22) Filed: Dec. 29, 2020

Related U.S. Application Data
(63) Continuation of application No. 16/311,598, filed on Dec. 19, 2018.
(60) Provisional application No. 62/353,101, filed on Jun. 22, 2016.

(51) Int. Cl. C07K 7/08; C07K 7/50; C07K 7/06

(52) U.S. Cl. CPC C07K 7/08; C07K 7/06; C07K 7/50

(57) ABSTRACT

Cyclic peptide compounds are provided that can bind to one or more homologs of Hedghehog signaling proteins and block their interaction with the Patched receptor. These compounds are useful for suppressing Hedgehog-dependent stimulation of the Hedgehog pathway in cells and in living organisms. Methods for using the cyclic peptide compounds, and compositions comprising them, for treatment of cancers and other diseases which are characterized by aberrant Hedgehog-dependent activation of the Hedgehog pathway and/or which can benefit from chemical suppression of Hedgehog-dependent signaling are also disclosed.

### CROSS-REFERENCE TO RELATED APPLICATIONS

This application is a continuation of U.S. patent application Ser. No. 16/311,598, filed on Dec. 19, 2018, which is a national stage application under 35 U.S.C. § 371 of PCT Application No. PCT/US2017/038559, filed Jun. 21, 2017, which claims priority to and the benefit of U.S. provisional patent application Ser. No. 62/353,101 entitled Cyclic Peptide Inhibitors of Hedgehog Proteins, filed Jun. 22, 2016. These applications are incorporated herein by reference in their entirety.

### STATEMENT REGARDING FEDERALLY SPONSORED RESEARCH OR DEVELOPMENT

This invention was made with government support under CA187502 awarded by the the National Institutes of Health. The government has certain rights in the invention.

### 1. TECHNICAL FIELD

The present invention relates to macrocyclic peptides that bind to Hedgehog proteins (Sonic Hedgehog, Indian Hedgehog, Desert Hedgehog) and are able to inhibit Hedgehog protein mediated stimulation of the Hedgehog signaling pathway. These compounds are useful for suppressing the activity of the Hedgehog signaling pathway in cells and in the treatment of human diseases and conditions that are associated with physiological or aberrant ligand mediated activation of the Hedgehog pathway. Methods for using the macrocyclic peptides, and compositions comprising them, for treating such conditions and diseases, which include proliferative diseases, inflammatory diseases, and developmental disorders, are also disclosed.

### 2. BACKGROUND

The Hedgehog (Hh) signaling pathway (FIG. 1) covers a central role during embryonic development controlling cell growth and differentiation, tissue patterning, and organogenesis (Chiang, Ying et al. 1996; Ingham and McMahon 2001; Ingham and Placzek 2006). Stimulation of the Hedgehog pathway is mediated by a complex sequence of molecular events at the level of the membrane and primary cilia of vertebrate cells, resulting in an intracellular signaling cascade and transcriptional response (Ingham and McMahon 2001; Jiang and Hui 2008; Wong and Reiter 2008). Canonical activation of this pathway is initiated by binding of the Hedgehog signaling proteins (i.e., Sonic (Shh), Indian (Ihh), and/or Desert (Dhh) Hedgehog), to the extracellular domain of the 12-pass transmembrane Patched (PTCH1) receptor (Carpenter, Stone et al. 1998). This event relieves Patched-mediated inhibition on the 7-pass transmembrane Smoothened (Smo) receptor, allowing Smo to translocate from the plasma membrane and endoplasmic vesicles to the primary cilium (Denef, Neubuser et al. 2000). Smo activation results in the accumulation of the active forms of Gli2 and Gli3 transcription factors (Kim, Kato et al. 2009; Wen, Lai et al. 2010), which stimulate the transcription of Gli-controlled genes, including Gli1 and PTCH1 (Yoon, Kita et al. 2002; Kasper, Regl et al. 2006). Aberrant activation of the Hedgehog pathway has been associated with tumorigenesis in several human tissues (Rubin and de Sauvage 2006; Theunissen and de Sauvage 2009). In particular, an increasing number of tumors, including leukemia (Dierks, Beigi et al. 2008; Hegde, Peterson et al. 2008; Zhao, Chen et al. 2009), small-cell lung (Watkins, Berman et al. 2003), pancreas (Thayer, di Magliano et al. 2003), and colon (Berman, Karhadkar et al. 2003) cancer, have been found to rely on ligand-dependent Hedgehog signaling for sustained growth and proliferation. Hh signaling is also implicated in the maintenance and propagation of cancer stem cells (Liu, Dontu et al. 2006; Dierks, Beigi et al. 2008; Zhao, Chen et al. 2009), which are believed to play a crucial role in tumor self-renewal, survival against chemotherapy, and metastasis (Reya, Morrison et al. 2001; Beachy, Karhadkar et al. 2004). Beside cancer, the development or progression of other human pathologies have been associated with stimulation of the Hedgehog pathway.

Because of the therapeutic potential of targeting the Hedgehog pathway, important efforts have been devoted to the development of chemical modulators of this signaling pathway (Peukert and Miller-Moslin 2010; Stanton and Peng 2010; Sharpe, Wang et al. 2015). These efforts have led to the identification of several potent inhibitors of the Smo receptor (Peukert and Miller-Moslin 2010; Stanton and Peng 2010; Sharpe, Wang et al. 2015), including cyclopamine (Cooper, Porter et al. 1998) and vismodegib (Robarge, Brunton et al. 2009), which represent the archetypal member and first FDA-approved drug, respectively, belonging to this class of Hh pathway antagonists. Compounds that target other downstream components of this pathway (Peukert and Miller-Moslin 2010; Stanton and Peng 2010; Sharpe, Wang et al. 2015) have also been reported. In stark contrast, potent inhibitors of the Shh/Patched protein-protein interaction have remained elusive. To date, the only compound known to target this interaction is Robotnikinin and derivatives thereof (Stanton, Peng et al. 2009) (U.S. Pat. No. 8,530,456 by Schreiber et al.). These molecules are however significantly less potent as Hedgehog pathway antagonists than the Smo inhibitors currently under investigation in clinical trials (Peukert and Miller-Moslin 2010; Stanton and Peng 2010; Sharpe, Wang et al. 2015), possibly reflecting the challenge of disrupting protein-protein interactions with small-molecule compounds. In addition, our own studies show that Robotnikinin has significantly lower affinity for Dhh and Ihh compared to Shh. While Shh is the best studied ligand of the Hedgehog pathway, paracrine and/or autocrine stimulation of Hedgehog signaling in normal and cancer cells is also mediated by the Indian (Ihh) and Desert (Dhh) homologs (Azoulay, Terry et al. 2008; Ibuki, Ghaffari et al. 2013). Hedgehog-targeted inhibitors capable of targeting multiple homologs of Hedgehog are thus required for effective suppression of ligand-induced activation of this pathway. On the other hand, anti-Hedgehog antibodies have been useful in investigating the therapeutic potential of disrupting the Hedgehog/Patched interaction in animal models of leukemia and other cancers (Dierks, Grbic et al. 2007; Yauch, Gould et al. 2008). However, the development, manufacturing, and application of monoclonal antibodies as therapeutics are faced with numerous challenges and limitations (Chames, Van Regenmortel et al. 2009).

Therefore, chemical compounds that can potently bind to and inhibit the signaling activity of Hedgehog proteins are currently missing. These compounds would have several important applications, including their use for the pharmacological treatment of pathologies and conditions associated with aberrant activity or activation of the Hedgehog pathway. These molecules would be also useful as chemical probes for detection, capturing, and imaging of Hedgehog proteins in vitro, in tissues, and in living organisms. The methods and compositions described herein provide a solution to this need.

Citation or identification of any reference in Section 2, or in any other section of this application, shall not be considered an admission that such reference is available as prior art to the present invention.

### 3. SUMMARY

A cyclic peptide is provided of Formula (I) or Formula (II), or a pharmaceutically acceptable salt thereof:

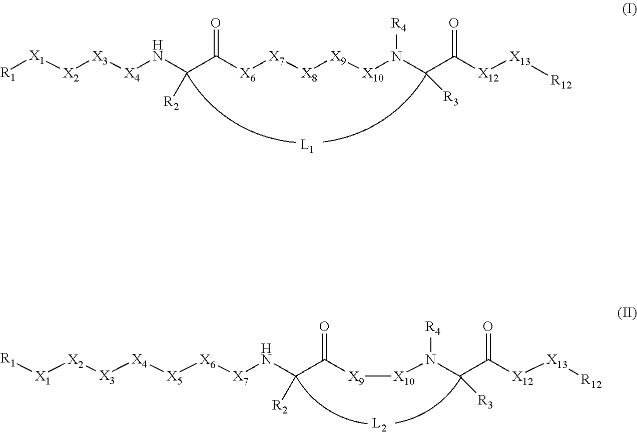

wherein:
   X₁ is Thr, Asp, Phe, Glu, Asn, Gln, Ser, Tyr, substituted Phe, substituted Tyr, or substituted Trp;
   X₂ is Leu, His, Ser, Val, Trp, Ile, Phe, Tyr, Thr, Nval, Nleu, substituted Phe, substituted Tyr, or substituted Trp;
   X₃ is Asp, Ser, Lys, Thr, Val, Asn, Leu, Glu, Ile, Gln, Arg, Orn, Dab, Dap, Nleu, or Nval;
   X₄ is Asp, Trp, Gly, Ala, Pro, Tyr, Phe, Glu, Aib, substituted Phe, substituted Tyr, substituted Trp, or an amino acid of formula (IV)

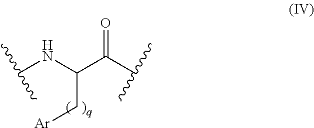

wherein:
   q is 0, 1, or 2; and Ar is a C₅-C₁₅ aryl group or a C₅-C₁₅ substituted aryl group;
   X₆ is Glu, Ser, Leu, Val, Trp, Gly, Ala, Arg, Tyr, Phe, Ile, Thr, Asp, Lys, Orn, Dab, Dap, substituted Phe, substituted Tyr, substituted Trp, Nval, or Nleu;
   X₇ is Glu, Ala, Lys, Gly, Tyr, Phe, Arg, Asp, Orn, Dab, Dap, substituted Phe, substituted Tyr, substituted Trp, Aib, 3,3,3-trifluoro-alanine, alpha,alpha-bis-trifluoromethyl-glycine, or 2-cyclopropylglycine;
   X₈ is Met, Nleu, Gly, or Pro;
   X₉ is Asp, Glu, Cys, His, or an amino acid residue of formula (III)

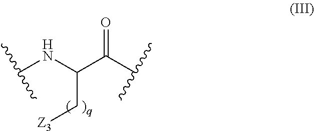

wherein:
q is 0, 1, or 2; and Z₃ is selected from the group consisting of —CONHOH, —N(OH)COCH₃, —CONHOCH₃, —ONHCOCH₃, —P(═O)(OH)₂, —P(═O)H(OH), —SO₃H, —SO₂NH₂, —NHSO₂CH₃, —CONHSO₂CH₃, —NHCONHSO₂CH₃, imidazole, 1,2,3-triazole, 1,2,4-triazole, tetrazole, —CH(OH)CF₃, —C(OH)₂CF₃, thiazolidine-2,4-dione, oxazolidine-2,4-dione, 1,2,4-oxadiazol-5(4H)-one, 1,2,4-thiadiazol-5(4H)-one, 3H-1,2,3,5-oxathiadiazole 2-oxide, 1,2,4-oxadiazole-5(4H)-thione, isoxazol-3-ol, isothiazol-3-ol, pyrrolidine-2,4-dione, furan-2,4(3H,5H)-dione, 3-hydroxycyclopent-2-enone, 3-hydroxycyclobut-3-ene-1,2-dione, and 2,6-difluorophenol;
X₁₀ is Gly, Met, D-Met, Nleu, D-Nleu, Thr, D-Thr, Ser, D-Ser, or —N(R₉)CH₂C(O)—, wherein R₉ is a C₁-C₁₅ aliphatic, C₁-C₁₅ substituted aliphatic, C₅-C₁₅ aryl, C₅-C₁₅ substituted aryl, C₆-C₁₅ alkylaryl, or C₆-C₁₅ substituted alkylaryl group;
X₁₂ is Ser, Thr, Met, Leu, Ile, Val, Nleu, or Nval;
X₁₃ is Asp, Glu, or absent;
X₅ is Met, Gly, or Nleu;
R₁ is hydrogen, an acetyl group, a label molecule, or a R₁₃CO— group, wherein R₁₃ is C₅-C₁₅ alkanoic acid or C₅-C₁₅ alkenoic acid;
R₂ and R₃ are, independently, a hydrogen or an alkyl group comprising between one and three carbon atoms;
R₄ is hydrogen, or a linear or branched alkyl group comprising between one and eight carbon atoms;
R₁₂ is —OH or NH₂;
L₁ is a linker unit, such that the linear dimension between the Cα carbon atoms connected by the linker unit is between about 6 and 15 Angstrom units; and
L₂ is a linker unit, such that the linear dimension between the Cα carbon atoms connected by the linker unit is between about 5 and 12 Angstrom units; and wherein the cyclic peptide is able to bind at least one homolog of a Hedgehog protein.

A cyclic peptide is provided of Formula (I), or a pharmaceutically acceptable salt thereof:

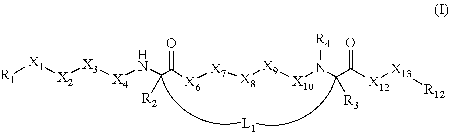

wherein:
   X₁ is Thr, Asp, Phe, Glu, Asn, Gln, Ser, or Tyr;
   X₂ is Leu, His, Ser, Val, Trp, Ile, Phe, Tyr, or Thr;
   X₃ is Asp, Ser, Lys, Thr, Val, Asn, Leu, Glu, Ile, Gln, Gly, Pro, Ala, Trp or Arg;
   X₄ is Asp, Trp, Gly, Ala, Pro, Tyr, Phe, or Glu;
   X₆ is Glu, Ser, Leu, Val, Trp, Gly, Ala, Arg, Tyr, Phe, Ile, Thr, Asp, or Lys;
   X₇ is Glu, Ala, Lys, Gly, Tyr, Phe, Arg, or Asp;
   X₈ is Met, Nleu, Gly, or Pro;
   X₉ is Asp or Glu;
   X₁₀ is Gly, Met, Nleu, Thr, or Ser;
   X₁₂ is Ser, Thr, Met, Leu, Ile, or Val;
   X₁₃ is Asp, Glu, or absent;
   R₁ is hydrogen, an acetyl group, or an acyl group;
   R₂ and R₃ are, independently, a hydrogen or an alkyl group comprising between one and three carbon atoms;
   R₄ is hydrogen, or a linear or branched alkyl group comprising between one and eight carbon atoms;
   R₁₂ is —OH or NH₂; and
   L₁ is a linker unit, such that the linear dimension between the Cα carbon atoms connected by the linker unit is between about 6 and 15 Angstrom units.

In an embodiment of the cyclic peptide, X₁ is Thr; X₂ is Leu, His, Ser, Val, or Trp; X₃ is Asp, or Ser; X₄ is Asp, Trp, or Gly; X₆ is Glu, Ser, Leu, Val, Trp, Gly, Ala, Arg, Tyr, or Phe; X₇ is Glu, Ala, Lys, or Gly; X₈ is Met; X₉ is Asp; X₁₀ is Gly, or Met; X₁₂ is Ser, or Thr; X₁₃ is Asp.

A cyclic peptide is provided of Formula (II), or a pharmaceutically acceptable salt thereof:

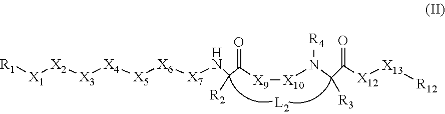

wherein:
   X₁ is Thr, Asp, Phe, Glu, Asn, Gln, Ser, or Tyr;
   X₂ is Leu, His, Ser, Val, Trp, Ile, Phe, Tyr, or Thr;
   X₃ is Asp, Ser, Lys, Thr, Val, Asn, Leu, Glu, Ile, Gln, or Arg;
   X₄ is Asp, Trp, Gly, Ala, Pro, Tyr, Phe, or Glu;
   X₅ is Met or Gly;
   X₆ is Glu, Ser, Leu, Val, Trp, Gly, Ala, Arg, Tyr, Phe, Ile, Thr, Asp, or Lys;
   X₇ is Glu, Ala, Lys, Gly, Tyr, Phe, Arg, or Asp;
   X₉ is Asp or Glu;
   X₁₀ is Gly, Met, Nleu, Thr, or Ser;
   X₁₂ is Ser, Thr, Met, Leu, Ile, or Val;
   X₁₃ is Asp, Glu, or absent;
   R₁ is hydrogen, an acetyl group, or an acyl group;
   R₂ and R₃ are, independently, a hydrogen or an alkyl group comprising between one and three carbon atoms;
   R₄ is hydrogen or an alkyl group comprising between one and eight carbon atoms;
   R₁₂ is —OH or NH₂; and
   L₂ is a linker unit, such that the linear dimension between the Cα carbon atoms connected by the linker unit is between about 5 and 12 Angstrom units.

In an embodiment of the cyclic peptide, X₁ is Thr; X₂ is Leu; X₃ is Asp; X₄ is Asp; X₅ is Met; X₆ is Glu; X₇ is Glu; X₈ is Met; X₉ is Asp; X₁₀ is Gly; X₁₂ is Ser, Leu, or Val; X₁₃ is Asp.

In an embodiment of the cyclic peptide, L₁ is —R₉—S—R₁₀— or —R₁₀—S—R₉—, wherein R₉ is an alkylaryl or a substituted alkylaryl group comprising between 5 and 20 carbon atoms, and R₁₀ is an alkyl or a substituted alkyl group comprising between 1 and 10 carbon atoms.

In an embodiment of the cyclic peptide, L₁ is

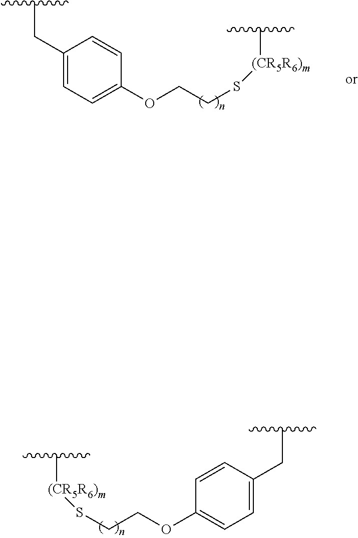

wherein n is an integer number comprised between 1 and 6; m is an integer number comprised between 1 and 3; and R₅ and R₆ are, independently, a hydrogen atom or a methyl group.

In an embodiment of the cyclic peptide, L₁ is —(CH₂)ₙ—Z₁—(CH₂)ₚ—, wherein Z₁ is —S—, —S—S—, —NHCO—, —CONH—, —CH═CH—, —(CH₂)₂—, —CH—CH—, or a triazole group, and n and p are, independently, an integer number between 1 and 6.

In an embodiment of the cyclic peptide, L₁ is —C(R₅R₆)ₘ-S—Z₂—S—C(R₇R₈)ₒ, wherein Z₂ is selected from the group consisting of a C₁-C₁₀ alkyl, C₁-C₁₀ substituted alkyl, C₂-C₁₀ alkenyl, C₂-C₁₀ substituted alkenyl, C₆-C₁₂ aryl, C₆-C₁₂ substituted aryl, C₅-C₁₄ alkylaryl, C₅-C₁₄ substituted alkylaryl group, —CH₂COCH₂—, and —CH₂COCOCH₂—; m and o are, independently, an integer number comprised between 1 and 3; R₅, R₆, R₇, and R₈ are, independently, a hydrogen atom or a methyl group.

In an embodiment of the cyclic peptide, L₂ is —R₉—S—R₁₀— or —R₁₀—S—R₉—, wherein R₉ is an alkylaryl or a substituted alkylaryl group comprising between 5 and 20 carbon atoms, and R₁₀ is an alkyl or a substituted alkyl group comprising between 1 and 10 carbon atoms.

In an embodiment of the cyclic peptide, L₂ is

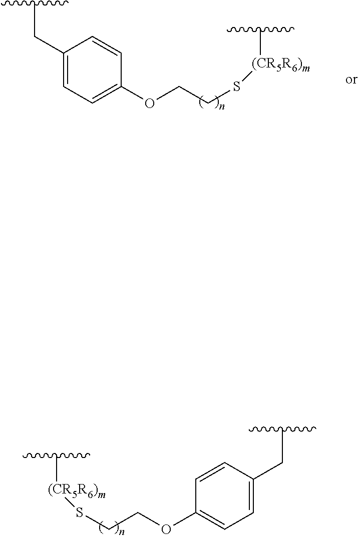

wherein n is an integer number comprised between 1 and 6; m is an integer number comprised between 1 and 3; and R₅ and R₆ are, independently, a hydrogen atom or a methyl group.

In an embodiment of the cyclic peptide, L₂ is —(CH₂)ₙ—Z₁—(CH₂)ₚ—, wherein Z₁ is —S—, —S—S—, —NHCO—, —CONH—, —CH═CH—, —(CH₂)₂—, —CH—CH—, or a triazole group, and n and p are, independently, an integer number between 1 and 6.

In an embodiment of the cyclic peptide, L₂ is —C(R₅R₆)ₘ—S—Z₂—S—C(R₇R₈)ₒ, wherein Z₂ is selected from the group consisting of a C₁-C₁₀ alkyl, C₁-C₁₀ substituted alkyl, C₂-C₁₀ alkenyl, C₂-C₁₀ substituted alkenyl, C₆-C₁₂ aryl, C₆-C₁₂ substituted aryl, C₈-C₁₄ alkylaryl, C₈-C₁₄ substituted alkylaryl group, —CH₂COCH₂—, and —CH₂COCOCH₂—; m and o are, independently, an integer number comprised between 1 and 3; R₅, R₆, R₇, and R₈ are, independently, a hydrogen atom or a methyl group.

In an embodiment of the cyclic peptide, at least one among residues X₁, X₂, X₃, X₄, X₅, X₆, and X₇ is an alpha,alpha-disubstituted amino acid carrying an alpha-methyl group.

In an embodiment of the cyclic peptide, at least one of residue X₁, X₂, X₁₀, X₁₁, X₁₂, and X₁₃ is an N-methylated amino acid.

In an embodiment of the cyclic peptide, the peptide comprises a warhead group. In another embodiment, the warhead group is —SO₂F, —Ar₂—SO₂F, —(CH₂)ₙSO₂F, —Ar₂—NCS, —NHSO₂CH═CH₂, or —(CH₂)ᵣCH₂Br, wherein Ar₂ is a C₅-C₁₀ aryl or a C₅-C₁₀ substituted aryl group; n is an integral number between 1 and 6; r is an integral number between 1 and 10.

In an embodiment of the cyclic peptide, L is —CH₂SSCH₂—, —CH₂SCH₂SCH₂—, —CH₂S(CH₂)₂SCH₂—, —CH₂S(CH₂)₃SCH₂—, —CH₂S(CH₂)₄SCH₂—, —CH₂S(CH₂)₅SCH₂—, —CH₂SCH₂CH═CHCH₂SCH₂CH₂—,

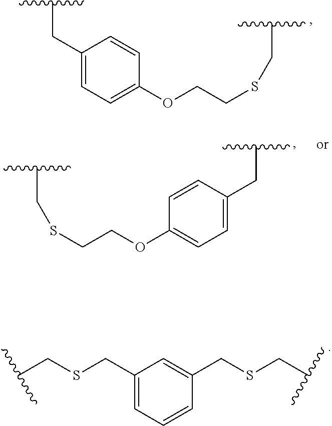 [SMILES: *CSCCOc1ccc(C*)cc1 ; *CSCCOc1ccc(C*)cc1 ; *CSCc1cccc(CSC*)c1]

In an embodiment of the cyclic peptide, the peptide is one selected from a peptide set forth in Tables 1, 2, 3 or 4.

In an embodiment of the cyclic peptide, the peptide comprises a label molecule selected from an affinity label molecule, a photoaffinity label, a dye, a chromophore, a fluorescent molecule, a phosphorescent molecule, a chemiluminescent molecule, an energy transfer agent, a photocrosslinker molecule, a redox-active molecule, an isotopic label molecule, a spin label molecule, a radioactive moiety, a contrast agent molecule, a MRI contrast agent, an isotopically labeled molecule, a PET agent, an electron dense group, a magnetic group, a cofactor, a biotin, a biotin analogue or a combination thereof.

A pharmaceutical composition is provided comprising a cyclic peptide disclosed herein and a pharmaceutically acceptable carrier.

A method is provided for treating a patient afflicted with cancer, comprising the step of administering to the patient a pharmaceutical composition comprising an effective amount of a cyclic peptide disclosed herein or a pharmaceutical composition comprising a cyclic peptide disclosed herein and a pharmaceutically acceptable carrier.

In an embodiment of the method, the cancer is leukemia or lymphoma, a pancreatic tumor, a tumor of the gastrointestinal tract, a hepatic carcinoma, a lung carcinoma, an ovarian carcinoma, a prostate carcinoma, a breast carcinoma, or a brain cancer.

A method is provided for treating a patient afflicted with a disease, whose ethiopathology is associated with Hedgehog-dependent stimulation of the Hedgehog pathway and/or whose treatment benefits from suppression of Hedgehog protein-dependent signaling, this method comprising the step of administering to the patient a pharmaceutical composition comprising an effective amount of a cyclic peptide of Claim 1 or a pharmaceutical composition of claim 21.

A method is provided for treating a patient afflicted with a disease, whose ethiopathology is associated with Hedgehog-dependent stimulation of the Hedgehog pathway and/or whose treatment benefits from suppression of Hedgehog protein-dependent signaling, this method comprising the step of administering to the patient a pharmaceutical composition comprising an effective amount of a cyclic peptide of Claim 1 or a pharmaceutical composition of claim 21.

### 4. BRIEF DESCRIPTION OF THE DRAWINGS

FIG. 1. Hedgehog signaling pathway. Binding of the Hh ligand(s) (Shh, Ihh, or Dhh) to the Patched receptor relieves its inhibitory effect on Smoothened (Smo), resulting in the activation of Gli transcription factors and induction of Gli-controlled genes. HHIP inhibits the signaling pathway by competing with Patched for binding to Hh ligands.

FIG. 2. Crystal structure of Shh in complex with the extracellular domain of HHIP (pdb 3HO5). The three loop regions of HHIP involved in Shh binding are labeled and the zinc ion in the L2 binding cleft of Shh is shown as a sphere model.

FIG. 3. Schematic structure of cyclic peptide HL2-m1 (top). Model of HL2-m1 (stick model) bound to Shh (surface model). The L2 loop of HHIP (ribbon model) is superimposed to the modeled HL2-m1/Shh complex. The peptide labelled “HL2-m1” shown in FIG. 3 corresponds to the homonymous peptide “HL2-m1” in Table 1 (second entry). The amino acid sequence thereof is provided in SEQ. ID. NO. 7.

FIG. 4. Optimization of cyclic peptide Hedgehog inhibitors. A plasmid library encoding for partially randomized peptide sequences fused to a FLAG tag and a C-terminal GyrA intein are transformed into E. coli cells and arrayed on multiwell plates. The corresponding precursor polypeptides are produced via ribosomal translation and O2beY incorporation via amber stop codon (TAG) suppression. The macrocyclic peptides are produced inside cells through ‘self-processing’ of the biosynthetic precursors via O2beY/Cys cyclization and aspartate-induced intein cleavage. After cell lysis, peptide binding to immobilized Shh is quantified colorimetrically. The variants showing improved Shh binding activity are deconvoluted via DNA sequencing. The best variant and acquired SAR data are used for the next round of affinity maturation.

FIG. 5. Shh binding affinity of linear L2-derived peptide (FLAG-HL2-pep=MDYKDDDDK-GSGS-TLDDMEEMDGLSDT) (SEQ ID NO: 6) and cyclic peptides. The plot shows dose-response curves for direct binding of the selected FLAG tag-fused peptides to plate-immobilized GST-Shh as determined using HRP-conjugated anti-FLAG antibody. The corresponding K_{D} values are reported in the table.

FIG. 6. Relative Shh binding activity for representative cyclic peptides from the single-site mutagenesis libraries. Shh binding activity was determined using the colorimetric assay with immobilized GST-Shh protein, FLAG tag-fused peptides, and HRP-conjugated anti-FLAG antibodies. The absorbance values (X axis) are normalized to that of cyclic peptide FLAG-HL2-m1. Indicated mutations (Y axis) are relative to the FLAG-HL2-m1 sequence according to numbering scheme shown in Table 1.

FIG. 7. Relative Shh binding activity for representative cyclic peptides from the multi-site mutagenesis libraries. Shh binding activity was determined using the colorimetric assay with immobilized GST-Shh protein, FLAG tag-fused peptides, and HRP-conjugated anti-FLAG antibodies. The absorbance values (X axis) are normalized to that of cyclic peptide FLAG-HL2-m1. Indicated mutations (Y axis) are relative to the FLAG-HL2-m1 sequence according to numbering scheme shown in Table 1.

FIG. 8. Relative Shh binding activity for representative cyclic peptides from HL2-m5-derived site-saturation mutagenesis libraries. Shh binding activity was determined using the colorimetric assay with immobilized GST-Shh protein, FLAG tag-fused peptides, and HRP-conjugated anti-FLAG antibodies. The absorbance values (X axis) are normalized to that of cyclic peptide FLAG-HL2-m5. Indicated mutations (Y axis) are relative to the FLAG-HL2-m5 sequence according to numbering scheme shown in Table 1.

FIG. 9. Shh binding affinity of X₅-X₈-linked cyclic peptides. The plot shows dose-response curves for direct binding of the selected FLAG tag-fused peptides to plate-immobilized GST-Shh as determined using HRP-conjugated anti-FLAG antibody. The corresponding K_{D} values are reported in the table.

FIG. 10. Dose-response curves for direct binding of FLAG-HL2-m5 to plate-immobilized GST-Shh, GST-Ihh, or GST-Dhh as determined using the colorimetric assay with HRP-conjugated anti-FLAG antibody. The corresponding K_{D} values are reported in the table.

FIG. 11. Suppression of Hedgehog pathway signaling in cells by the cyclic peptide HL2-m5. The graphs shows dose dependent inhibition of ligand-dependent Hedgehog pathway activation induced by the cyclic peptide HL2-m5 as determined using Shh-stimulated NIH-3T3 cells containing a dual luciferase reporter system. In contrast to the cyclic peptide, a linear peptide derived from the HHIP L2 loop (HL2-pep) is unable to suppress signaling through the Hedgehog pathway in the cells. Luminescence values were normalized to that of Shh-stimulated cells grown in the absence of the inhibitor.

FIG. 12. Restoration of Hedgehog pathway signaling with purmorphamine. These graph shows the relative extent of Hedgehog pathway activity in NIH-3T3 Gli activity reporter cells upon incubation with (i) Shh, (ii) Shh and the cyclic peptide inhibitor HL2-m5 (10 mM), and (iii) Shh, HL2-m5, and the Smo activator purmorphamine (5 mM). The luciferase activity response was normalized to that of cells stimulated with Shh in the absence of the cyclic peptide inhibitor and the Smo activator.

FIG. 13. Inhibition of Hedgehog-induced transcriptional response. The graph shows the relative transcriptional levels of the Hedgehog pathway target genes Gli, Gi2, and Ptch1 in Shh-stimulated NIH-3T3 cells in the presence and in the absence of HL2-m5 (10 mM) as determined by real-time PCR. The mRNA levels of the same genes in unstimulated cells are included for comparison.

FIG. 14. Hh analog selectivity for linear HHIP L2-derived peptide (HL2-pep) and representative cyclic peptide inhibitors. The data relative to the peptides were derived from direct binding experiments with immobilized Hedgehog proteins (i.e., GST-Shh, GST-Ihh, and GST-Dhh), biotinylated peptides (i.e., Biot-HL2-pep, Biot-HL2-m1, Biot-HL2-m2, Biot-HL2-m3, Biot-HL2-m4, Biot-HL2-m4), and HRP-conjugated streptavidin. The data relative to robotnikinin were derived from competition experiments (10 mM robotnikinin+400 nM HL2-m5). For each compound, the response values were normalized to the highest binding response measured across the three Hedgehog analogs.

FIG. 15. Model of evolved macrocyclic peptide HL2-m5 in complex with Shh. Shh protein is shown as a surface model, whereas the macrocyclic peptide is shown as a stick model. The N-terminal and C-terminal residues along with Trp4 and Met10 are labeled. According to the model, the side chains of Trp4 and Met10 establish contacts with the regions of the Shh surface that are not contacted by the L2 loop of HHIP.

FIG. 16. Synthetic route for the preparation of the diamino acid building block encompassing the linker unit L-1.

FIG. 17. Solid-phase synthesis of cyclic peptide HL2-m5. This procedures exemplifies a synthetic method for the preparation of a cyclic peptide of formula (I) or (II) wherein the linker unit corresponds to L-1. SPPS=solid phase peptide synthesis. The peptide labelled HL2-m5 in this Figure corresponds to the homonymous peptide “HL2-m5” in Table 1. The amino acid sequence thereof is provided in SEQ. ID. NO. 37.

FIG. 18. Solid-phase synthesis of cyclic peptide m5-s4. This procedure exemplifies a synthetic method for the preparation of a cyclic peptide of formula (I) or (II) wherein the linker unit corresponds to L-6. For simplicity, only the side-chain protecting group of the cysteine residues (Trt=trityl) is shown. SPPS=solid phase peptide synthesis. The peptide labelled m5-s4 in this Figure corresponds to the homonymous peptide “m5-s4” in Table 2. The amino acid sequence thereof is provided in SEQ. ID. NO. 5.

FIG. 19. Solid-phase synthesis of cyclic peptide m5-s4. This procedure exemplifies a synthetic method for the preparation of a cyclic peptide of formula (I) or (II) wherein the linker unit corresponds to L-6. For simplicity, only the side-chain protecting group of the cysteine residues (Mtt=4-methyltrityl) is shown. SPPS=solid phase peptide synthesis. The peptide labelled m5-s4 in this Figure corresponds to the homonymous peptide “m5-s4” in Table 2. The amino acid sequence thereof is provided in SEQ. ID. NO. 5.

FIG. 20. Solid-phase synthesis of cyclic peptide m5-s4.2. This procedure exemplifies a synthetic method for the preparation of a warhead-containing cyclic peptide of formula (I), wherein the linker unit corresponds to L-6 and the warhead group is a sulfonylfluoride group. For simplicity, only the side-chain protecting groups of the cysteine residues (Trt=trityl) and Dap residue (Alloc=allyloxycarbonyl) are shown. SPPS=solid phase peptide synthesis. The peptide labelled m5-s4.2 in this Figure corresponds to the homonymous peptide “m5-s4.2” in Table 4. The amino acid sequence thereof is provided in SEQ. ID. NO. 61.

### 5. DETAILED DESCRIPTION

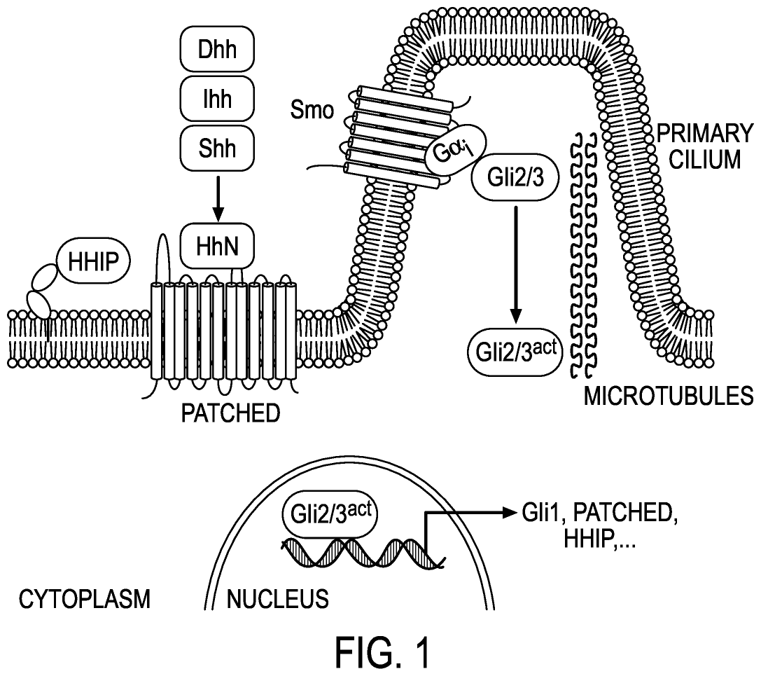

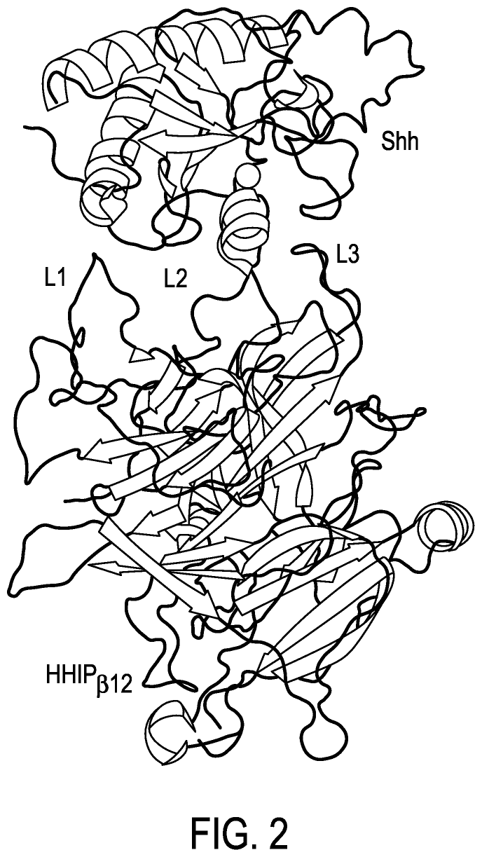

Canonical activation of the Hedgehog pathway initiates with the interaction of the Hedgehog protein ligands (Shh, Ihh, or Dhh) with the Patched receptor, resulting in the derepression of Smo receptor and consequent activation of Gli transcription factors and transcription of Gli-controlled target genes (FIG. 1). Hedgehog-interacting protein (HHIP) is a membrane protein that acts as a negative regulator of the Hedgehog pathway (Chuang and McMahon 1999). HHIP expression is upregulated in response to Hedgehog signaling (Chuang, Kawcak et al. 2003) and downregulated in various cancers (Tojo, Kiyosawa et al. 2002; Olsen, Hsu et al. 2004; Tada, Kanai et al. 2008). Recent crystallographic studies have provided insights into the structure of Shh in complex with HHIP (Bishop, Aricescu et al. 2009; Bosanac, Maun et al. 2009). In the crystallized complex, HHIP is found to interact with Shh primarily via an extended loop (L2) located in the extracellular domain of HHIP (FIG. 2). These studies also indicated that the Shh binding site involved in the interaction with HHIP L2 loop is shared by Patched, as suggested by the ability of a linear 18mer peptide derived from HHIP L2 loop to inhibit the Shh/Patched interaction in vitro, albeit with only very weak affinity (IC₅₀: 150 μM). The weak inhibitory activity of this peptide and other well-known limitations of linear peptides (i.e., proteolytic instability, limited cell permeability, rapid clearance in vivo, etc.) make this compound unsuitable for therapeutic purposes or as a probe for in vitro and/or in vivo studies. The inventors have instead discovered that small-size, cyclic peptides comprising part of the HHIP L2 sequence can provide high affinity binders for and potent inhibitors of the Hedgehog proteins. As disclosed herein, a new class of cyclic peptide compounds is provided. These cyclic peptide compounds are capable of binding and blocking the signaling properties of Hedgehog proteins, resulting in a suppression of Hedgehog protein-dependent activation of the Hedgehog pathway in a cell. In certain embodiments, provided compounds are useful to treat a proliferative disease, an inflammatory disease, a developmental disorder, or a neurodegenerative disease.

As used herein, the term “inhibitor” is defined as a compound that binds to and/or inhibits with measurable affinity at least one homolog of the Hedgehog protein, i.e., Shh, Ihh, and/or Dhh. In certain embodiments, an inhibitor has an IC₅₀ and/or binding constant of less about 50 μM, less than about 1 μM, less than about 500 nM, less than about 100 nM, less than about 10 nM, or less than about 1 nM.

Each of the cyclic peptide disclosed herein here can be viewed as a mimic of HHIP L2 loop, which corresponds to the 13mer sequence comprising residue 375 to residue 387 of HHIP primary sequence (TLDDMEEMDGLSD; SEQ ID NO: 1). As discussed in more details below, a cyclic peptide disclosed herein can bind to at least one homolog of Hedgehog proteins such as Shh, Ihh, and/or Dhh. In an embodiment, a cyclic peptide disclosed herein can bind more tightly to at least one homolog of Hedgehog than does a linear peptide derived from HHIP L2 loop. Furthermore, a cyclic peptide disclosed herein is able to inhibit Hedgehog ligand-induced stimulation of the Hedgehog signaling pathway in a cell. In an embodiment, a cyclic peptide disclosed herein is able to inhibit Hedgehog ligand-induced stimulation of the Hedgehog signaling pathway in a cell more effectively than a linear peptide derived from HHIP L2 loop.

For clarity of disclosure, and not by way of limitation, the detailed description is divided into the subsections set forth below.

### 5.1 Definitions

Unless defined otherwise herein, all technical and scientific terms used herein have the same meaning as commonly understood by one of ordinary skill in the art to which the disclosure pertains.

The singular forms “a,” “an,” and “the” used herein include plural referents unless the content clearly dictates otherwise.

The term “plurality” includes two or more referents unless the content clearly dictates otherwise.

The term “functional group” as used herein refers to a contiguous group of atoms that, together, may undergo a chemical reaction under certain reaction conditions. Examples of functional groups are, among many others, —OH, —NH₂, —SH, —(C═O)—, —N₃, —C≡CH.

The term “aliphatic” or “aliphatic group” as used herein means a straight or branched C₁₋₁₅ hydrocarbon chain that is completely saturated or that contains at least one unit of unsaturation, or a monocyclic C₃₋₈ hydrocarbon, or bicyclic C₈₋₁₂ hydrocarbon that is completely saturated or that contains at least one unit of unsaturation, but which is not aromatic (also referred to herein as “cycloalkyl”). For example, suitable aliphatic groups include, but are not limited to, linear or branched alkyl, alkenyl, alkynyl groups or hybrids thereof such as (cycloalkyl)alkyl, (cycloalkenyl)alkyl, or (cycloalkynyl)alkyl. The alkyl, alkenyl, or alkynyl group may be linear, branched, or cyclic and may contain up to 15, up to 8, or up to 5 carbon atoms. In an embodiment, an alkyl group can include, but not be limited to, a methyl, ethyl, propyl, cyclopropyl, butyl, cyclobutyl, pentyl, or cyclopentyl group. In an embodiment, an alkenyl group can include, but not be limited to, a propenyl, butenyl, or pentenyl group. In an embodiment, an alkynyl groups can include, but not be limited to, a propynyl, butynyl, or pentynyl group.

As used herein, the term “acyl” refers to a group R—C(O)— of from 1 to 10 carbon atoms of a straight, branched, or cyclic configuration or a combination thereof, attached to the parent structure through carbonyl functionality. Such group may be saturated or unsaturated, and aliphatic or aromatic. In an embodiment, R in the acyl residue is alkyl, or alkoxy, or aryl, or heteroaryl. In an embodiment, at least one carbon in the acyl residue may be replaced by nitrogen, oxygen or sulfur as long as the point of attachment to the parent remains at the carbonyl. Examples include, but are not limited to, acetyl, benzoyl, propionyl, isobutyryl, t-butoxycarbonyl, benzyloxycarbonyl and the like. Lower acyl refers to acyl containing one to four carbons.

The term “aryl” and “aryl group” as used herein refers to an aromatic substituent containing a single aromatic or multiple aromatic rings that are fused together, directly linked, or indirectly linked (such as linked through a methylene or an ethylene moiety). In an embodiment, an aryl group may contain from 5 to 24 carbon atoms, 5 to 18 carbon atoms, or 5 to 14 carbon atoms.

The term “alkylaryl” as used herein refers to an aryl group connected to an alkyl group via carbon-carbon bond or a carbon-heteroatom bond.

The terms “heteroatom” means nitrogen, oxygen, or sulphur, and includes any oxidized forms of nitrogen and sulfur, and the quaternized form of any basic nitrogen. Heteroatoms further include Se, Si, and P.

The term “heteroaryl” as used herein refer to an aryl group in which at least one carbon atom is replaced with a heteroatom. In an embodiment, a heteroaryl group is a 5- to 18-membered, a 5- to 14-membered, or a 5- to 10-membered aromatic ring system containing at least one heteroatom selected from the group consisting of oxygen, sulphur, and nitrogen atoms. Heteroaryl groups include, but are not limited to, pyridyl, pyrrolyl, furyl, thienyl, indolyl, isoindolyl, indolizinyl, imidazolyl, pyridonyl, pyrimidyl, pyrazinyl, oxazolyl, thiazolyl, purinyl, quinolinyl, isoquinolinyl, benzofuranyl, and benzoxazolyl groups.

A heterocyclic group may be any monocyclic or polycyclic ring system which contains at least one heteroatom and may be unsaturated or partially or fully saturated. The term “heterocyclic” thus includes heteroaryl groups as defined above as well as non-aromatic heterocyclic groups. In an embodiment, a heterocyclic group is a 3- to 18-membered, a 3- to 14-membered, or a 3- to 10-membered, ring system containing at least one heteroatom selected from the group consisting of oxygen, sulphur, and nitrogen atoms. In an embodiment, a heterocyclic group includes, but is not limited to, the specific heteroaryl groups listed above as well as pyranyl, piperidinyl, pyrrolidinyl, dioaxanyl, piperazinyl, macrocyleolinyl, thiomacrocyleolinyl, macrocyleolinosulfonyl, tetrahydroisoquinolinyl, and tetrahydrofuranyl groups.

A halogen atom may be a fluorine, chlorine, bromine, or iodine atom.

By “substituted” or “optionally substituted”, it is intended that in the any of the chemical groups listed above (e.g., alkyl, alkenyl, alkynyl, heteroalkyl, heteroalkenyl, heteroalkynyl, aryl, heteroaryl, heterocyclic, triazolyl groups), at least one hydrogen atom is optionally replaced with an atom or chemical group other than hydrogen. Specific examples of such substituents include, without limitation, halogen atoms, hydroxyl (—OH), sulfhydryl (—SH), substituted sulfhydryl, carbonyl (—CO—), carboxy (—COOH), amino (—NH₂), nitro (—NO₂), sulfo (—SO₂—OH), cyano (—C≡N), thiocyanato (—S—C≡N), phosphono (—P(O)OH₂), alkyl, alkenyl, alkynyl, heteroalkyl, heteroalkenyl, heteroalkynyl, aryl, heteroaryl, heterocyclic, alkylthiol, alkyloxy, alkylamino, arylthiol, aryloxy, or arylamino groups. Where “optionally substituted” modifies a series of groups separated by commas (e.g., “optionally substituted AA, BB, or CC”; or “AA, BB, or CC optionally substituted with”), it is intended that each of the groups (e.g., AA, BB, or CC) is optionally substituted.

The term “heteroatom-containing aliphatic” as used herein refer to an aliphatic moiety where at least one carbon atom is replaced with a heteroatom, e.g., oxygen, nitrogen, sulphur, selenium, phosphorus, or silicon, and typically oxygen, nitrogen, or sulphur.

The terms “alkyl” and “alkyl group” as used herein refer to a linear, branched, or cyclic saturated hydrocarbon typically containing 1 to 24 carbon atoms or 1 to 12 carbon atoms, such as methyl, ethyl, n-propyl, isopropyl, n-butyl, isobutyl, t-butyl, octyl, decyl and the like.

The term “heteroatom-containing alkyl” as used herein refers to an alkyl moiety where at least one carbon atom is replaced with a heteroatom, e.g., oxygen, nitrogen, sulphur, phosphorus, or silicon, and typically oxygen, nitrogen, or sulphur.

The terms “alkenyl” and “alkenyl group” as used herein refer to a linear, branched, or cyclic hydrocarbon group of 2 to 24 carbon atoms or, in an embodiment, of 2 to 12 carbon atoms, containing at least one double bond, such as ethenyl, n-propenyl, isopropenyl, n-butenyl, isobutenyl, octenyl, decenyl, and the like.

The term “heteroatom-containing alkenyl” as used herein refer to an alkenyl moiety where at least one carbon atom is replaced with a heteroatom.

The terms “alkynyl” and “alkynyl group” as used herein refer to a linear, branched, or cyclic hydrocarbon group of 2 to 24 carbon atoms or, in an embodiment, of 2 to 12 carbon atoms, containing at least one triple bond, such as ethynyl, n-propynyl, and the like.

The term “heteroatom-containing alkynyl” as used herein refer to an alkynyl moiety where at least one carbon atom is replaced with a heteroatom.

The term “heteroatom-containing aryl” as used herein refer to an aryl moiety where at least one carbon atom is replaced with a heteroatom.

The terms “alkoxy” and “alkoxy group” as used herein refer to an aliphatic group or a heteroatom-containing aliphatic group bound through a single, terminal ether linkage. In embodiments, aryl alkoxy groups can contain 1 to 24 carbon atoms, and in an embodiment, contain 1 to 14 carbon atoms.

The terms “aryloxy” and “aryloxy group” as used herein refer to an aryl group or a heteroatom-containing aryl group bound through a single, terminal ether linkage. Aryloxy groups can contain 5 to 24 carbon atoms, and in an embodiment, contain 5 to 14 carbon atoms.

The term “substituents” refers to a contiguous group of atoms. Examples of “substituents” include, without limitation: alkoxy, aryloxy, alkyl, heteroatom-containing alkyl, alkenyl, heteroatom-containing alkenyl, alkynyl, heteroatom-containing alkynyl, aryl, heteroatom-containing aryl, alkoxy, heteroatom-containing alkoxy, aryloxy, heteroatom-containing aryloxy, halo, hydroxyl (—OH), sulfhydryl (—SH), substituted sulfhydryl, carbonyl (—CO—), thiocarbonyl, (—CS—), carboxy (—COOH), amino (—NH₂), substituted amino, nitro (—NO₂), nitroso (—NO), sulfo (—SO₂—OH), cyano (—C≡N), cyanato (—O—C≡N), thiocyanato (—S—C≡N), formyl (—CO—H), thioformyl (—CS—H), phosphono (—P(O)OH₂), substituted phosphono, and phospho (—PO₂).

The term “cyclic” and “macrocyclic” as used herein means having constituent atoms forming a ring. Thus, a “cyclic peptide” is a peptide molecule that contains at least one ring formed by atoms comprised in the molecule. “Cyclization” or “macrocyclization” as used herein refers to a process or reaction whereby a cyclic molecule is formed or is made to be formed.

The term “α-amino acid” or simply “amino acid” refers to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. Unless the context specifically indicates otherwise, the term amino acid, as used herein, is intended to include amino acid analogs.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The term “amino acid analog” refers to a molecule which is structurally similar to an amino acid and which can be substituted for an amino acid in the formation of a peptide or peptidomimetic macrocycle. Amino acid analogs include compounds which are structurally identical to an amino acid, as defined herein, except for the inclusion of at least one additional methylene group between the amino and carboxyl group (e.g., α-amino O-carboxy acids), or for the substitution of the amino or carboxy group by a similarly reactive group (e.g., substitution of the primary amine with a secondary or tertiary amine, or substitution or the carboxy group with an ester).

The term “amino acid side chain” refers to a moiety attached to the α-carbon in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an α,α di-substituted amino acid).

The term “isostere” as used herein refers to two or more molecules that share at least one physico-chemical property. Physical chemical properties that can shared by two or more isostere molecules include, but are not limited to, H-bonding properties, acidity constant (pKₐ), basicity constant (pK_{b}), molecular weight, molecular surface area, molecular volume, length of the major molecular axis, water/octane partition coefficient, and the like.

The terms “peptide” and “polypeptide” as used herein refers to any chain of two or more naturally or non-naturally-occurring amino acids joined by a covalent bond (e.g., an amide bond. Polypeptides as described herein include full length proteins (e.g., fully processed proteins) as well as shorter amino acids sequences (e.g., fragments of naturally occurring proteins or synthetic polypeptide fragments).

The term “fatty acid” or “fatty acid molecule” refers to a terminal carboxylic acid containing a straight aliphatic chain consisting of eight to 22 carbon atoms and containing zero, 1, or two carbon-carbon double bonds. Fatty acid molecules can be also described as C₅-C₂₂ alkan-1-oic acids or C₈-C₂₂ alken-1-oic acids. Representative examples of fatty acid molecules include, but are not limited to, laurate, myristate, palmitate, stearate, arachidate, palmitoleate, oleate, linoleate, linolenate, and arachidonate. The term “fatty acid-like molecule” as used herein refers to a linear or branched terminal alkanoic acid or to a linear or branched terminal alkenoic acids comprising between 3 and 22 carbon atoms.

The terms “label molecule” or “tag molecule” as used herein refer to a molecule that allows detection of or monitoring of the structural changes in another molecule covalently bound to it (e.g., a target polypeptide) by physical detection methods. Examples of physical detection methods include, but are not limited to, mass spectrometry, UV absorbance, fluorescence, luminescence, PET, radiography, circular dichroism, nuclear magnetic resonance, and the like. The terms “affinity label molecule” or “affinity tag” as used herein refer to a molecule that allows for the isolation of another molecule covalently bound to it (e.g., a target polypeptide) by physical methods. Examples of physical methods include, but are not limited to, affinity chromatography, reverse-phase chromatography, ion-exchange chromatography, gel-permeation chromatography, and related techniques. The term “photoaffinity label,” as used herein, refers to a label molecule with a functional group, which, upon exposure to light, forms a linkage with a molecule for which the label molecule has an affinity. By way of example only, such a linkage may be covalent or non-covalent.

The term “dye,” as used herein, refers to a soluble, coloring substance that comprises a chromophore. The term “chromophore,” as used herein, refers to a molecule that absorbs light of visible wavelengths, UV wavelengths or IR wavelengths. The term “fluorescent molecule” as used herein refers to a molecule which upon excitation emits photons and is thereby fluorescent. The term “chemiluminescent molecule” as used herein refers to a molecule that emits light as a result of a chemical reaction without the addition of heat. The term “energy transfer agent,” as used herein, refers to a molecule that can either donate or accept energy from another molecule.

The term “photocrosslinker,” as used herein, refers to a compound comprising two or more functional groups which, upon exposure to light, are reactive and form a covalent or non-covalent linkage with two or more monomeric or polymeric molecules.

The term “redox-active agent,” as used herein, refers to a molecule that oxidizes or reduces another molecule, whereby the redox active agent becomes reduced or oxidized. Examples of redox active agent include, but are not limited to, ferrocene, quinones, Ru^{2+/3+} complexes, Co^{2+/3+} complexes, and Os^{2+/3+} complexes.

The term “spin label,” as used herein, refers to molecules that comprise an atom or a group of atoms exhibiting an unpaired electron spin (i.e., a stable paramagnetic group) that can be detected by electron spin resonance spectroscopy and can be attached to another molecule. Such spin-label molecules include, but are not limited to, nitryl radicals and nitroxides, and may be single spin-labels or double spin-labels.

The term “heavy atom,” as used herein, refers to an atom that is usually heavier than carbon. Such ions or atoms include, but are not limited to, silicon, tungsten, gold, lead, and uranium.

The term “radioactive moiety,” as used herein, refers to a group whose nuclei spontaneously release nuclear radiation, such as alpha, or beta particles, or gamma radiation.

The term “contrast agent” as used herein refers to a molecule that can be visualized, typically in the context of a biological tissue or organism, by means of physical detection methods. The term “MRI contrast agent” as used herein refers to a molecule that can be visualized, typically in biological tissue or organism, by means of magnetic resonance imaging (MRI). An example of an MRI contrast agents are gadolinium-based complexes and the like. The term “PET agent” as used herein refers to a molecule that can be visualized, typically in biological tissue or organism, by means of positron emission tomography (PET).

The term “photocaged moiety,” as used herein, refers to a group that, upon illumination at certain wavelengths, covalently or non-covalently binds ions or other molecules. The term “photoisomerizable moiety,” as used herein, refers to a group wherein upon illumination with light changes from one isomeric form to another.

The term “chemically cleavable group” as used herein refers to a functional group that breaks or cleaves upon exposure to acid, base, oxidizing agents, reducing agents, chemical initiators, or radical initiators. The term “photocleavable group” as used herein refers to a functional group that breaks or cleaves upon exposure to light.

The term “electron dense group,” as used herein, refers to a group that scatters electrons when irradiated with an electron beam. Such groups include, but are not limited to, ammonium molybdate, bismuth subnitrate cadmium iodide, carbohydrazide, ferric chloride hexahydrate, hexamethylene tetramine, and potassium ferricyanide.

The term “antibody fragment” as used herein refers to any form of an antibody other than the full-length form. Antibody fragments include but are not limited to Fv, Fc, Fab, and (Fab′)₂, single chain Fv (scFv), diabodies, combinations of CDRs, heavy chains, or light chains, bispecific antibodies, and the like.

The term “biotin analogue,” or also referred to as “biotin mimic,” as used herein, is any molecule, other than biotin, that binds with high affinity to avidin and/or streptavidin.

The term “isotopically labeled molecule” as used herein refers to a molecule that contains an enriched amount of a specific isotope of (a) certain atom(s) within the molecule as compared to the normal isotopic distribution. Example of “isotopically labeled molecules” include, but are not limited to, molecules comprising enriched amounts of ²H, ³H, ¹³C, ¹⁴N, ¹⁸F, and the like.

The term “polymer,” as used herein, refers to a molecule composed of repeated subunits. Such molecules include, but are not limited to, proteins, polypeptides, peptides, polynucleotides, polysaccharides, polyalkylene glycols, polyethylene, and polystyrene. As used herein, the term “water soluble polymer” refers to any polymer that is soluble in aqueous solvents. By way of example only, coupling of a water soluble polymer to a cyclic peptide provided herein may result in changes including, but not limited to, increased water solubility, increased or modulated serum half-life, increased bioavailability, modulated biological activity, extended circulation time, and modulated immunogenicity and modulated physical association characteristics including, but not limited to, aggregation and multimer formation and altered receptor affinity.

The term “biologically active molecule” as used herein refers to any molecule that can affect any physical or biochemical properties of a biological system, pathway, molecule, or interaction relating to an organism, including but not limited to, viruses, bacteria, bacteriophage, transposon, prion, insects, fungi, plants, animals, and humans. Examples of biologically active molecules include, but are not limited to, peptides, proteins, DNA, RNA, small-molecule drugs, polysaccharides, carbohydrates, lipids, radionuclides, toxins, cells, viruses, liposomes, microparticles and micelles.

The term “drug” as used herein refers to any substance used in the prevention, diagnosis, alleviation, treatment, or cure of a disease or condition.

The term “cytotoxic” as used herein, refers to a compound that harms cells.

The term “solid support” is used in the commonly accepted meaning to indicate any solid inorganic or organic, polymeric or non-polymeric material onto which a given molecule can be covalently or non-covalently bound so that the molecule is immobilized onto the solid support.

The term “resin” as used herein refers to high molecular weight, insoluble polymer beads. By way of example only, such beads may be used as supports for solid phase peptide synthesis, or sites for attachment of molecules prior to purification.

The term “nanoparticle” as used herein refers to a particle that has a particle size between about 500 nm (i.e., 500 nm±10%) to about 1 nm (i.e., 1 nm±10%).

The term “contact” as used herein with reference to interactions of chemical units indicates that the chemical units are at a distance that allows short range non-covalent interactions (such as Van der Waals forces, hydrogen bonding, hydrophobic interactions, electrostatic interactions, dipole-dipole interactions) to dominate the interaction of the chemical units. For example, when a protein is ‘contacted’ with a chemical species, the protein is allowed to interact with the chemical species so that a reaction between the protein and the chemical species can occur.

### 5.2 Cyclic Peptides

In one aspect, a cyclic peptide of Formula (I) & (II) is provided:
1. A cyclic peptide of Formula (I) or Formula (II), or a pharmaceutically acceptable salt thereof:

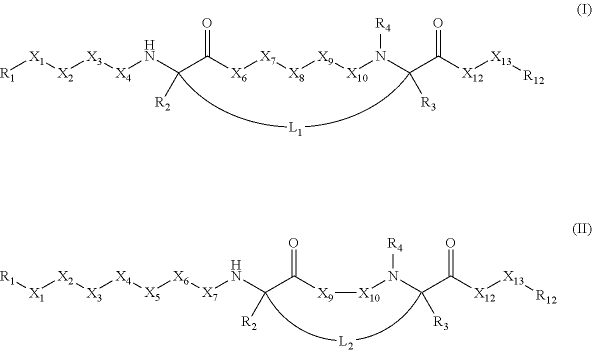

wherein:
   X₁ is Thr, Asp, Phe, Glu, Asn, Gln, Ser, Tyr, substituted Phe, substituted Tyr, or substituted Trp;
   X₂ is Leu, His, Ser, Val, Trp, Ile, Phe, Tyr, Thr, Nval, Nleu, substituted Phe, substituted Tyr, or substituted Trp;
   X₃ is Asp, Ser, Lys, Thr, Val, Asn, Leu, Glu, Ile, Gln, Arg, Orn, Dab, Dap, Nleu, or Nval;
   X₄ is Asp, Trp, Gly, Ala, Pro, Tyr, Phe, Glu, Aib, substituted Phe, substituted Tyr, substituted Trp, or an amino acid of formula (IV)

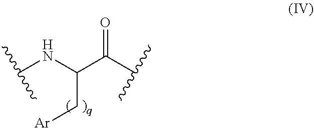

wherein:
   q is 0, 1, or 2; and Ar is a C₅-C₁₅ aryl group or a C₅-C₁₅ substituted aryl group;
   X₆ is Glu, Ser, Leu, Val, Trp, Gly, Ala, Arg, Tyr, Phe, Ile, Thr, Asp, Lys, Orn, Dab, Dap, substituted Phe, substituted Tyr, substituted Trp, Nval, or Nleu;
   X₇ is Glu, Ala, Lys, Gly, Tyr, Phe, Arg, Asp, Orn, Dab, Dap, substituted Phe, substituted Tyr, substituted Trp, Aib, 3,3,3-trifluoro-alanine, alpha,alpha-bis-trifluoromethyl-glycine, or 2-cyclopropylglycine;
   X₈ is Met, Nleu, Gly, or Pro;
   X₉ is Asp, Glu, Cys, His, or an amino acid residue of formula (III)

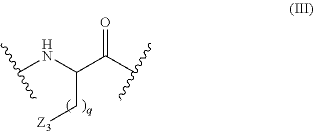

wherein:
   q is 0, 1, or 2; and Z₃ is selected from the group consisting of —CONHOH, —N(OH)COCH₃, —CONHOCH₃, —ONHCOCH₃, —P(═O)(OH)₂, —P(═O)H(OH), —SO₃H, —SO₂NH₂, —NHSO₂CH₃, —CONHSO₂CH₃, —NHCONHSO₂CH₃, imidazole, 1,2,3-triazole, 1,2,4-triazole, tetrazole, —CH(OH)CF₃, —C(OH)₂CF₃, thiazolidine-2,4-dione, oxazolidine-2,4-dione, 1,2,4-oxadiazol-5(4H)-one, 1,2,4-thiadiazol-5(4H)-one, 3H-1,2,3,5-oxathiadiazole 2-oxide, 1,2,4-oxadiazole-5(4H)-thione, isoxazol-3-ol, isothiazol-3-ol, pyrrolidine-2,4-dione, furan-2,4(3H,5H)-dione, 3-hydroxycyclopent-2-enone, 3-hydroxycyclobut-3-ene-1,2-dione, and 2,6-difluorophenol;
   X₁₀ is Gly, Met, D-Met, Nleu, D-Nleu, Thr, D-Thr, Ser, D-Ser, or —N(R₉)CH₂C(O)—, wherein R₉ is a C₁-C₁₅ aliphatic, C₁-C₁₅ substituted aliphatic, C₅-C₁₅ aryl, C₅-C₁₅ substituted aryl, C₆-C₁₅ alkylaryl, or C₆-C₁₅ substituted alkylaryl group;
   X₁₂ is Ser, Thr, Met, Leu, Ile, Val, Nleu, or Nval;
   X₁₃ is Asp, Glu, or absent;
   X₅ is Met, Gly, or Nleu;
   R₁ is hydrogen, an acetyl group, a label molecule, or a R₁₃CO— group, wherein R₁₃ is C₅-C₁₅ alkanoic acid or C₅-C₁₅ alkenoic acid;
   R₂ and R₃ are, independently, a hydrogen or an alkyl group comprising between one and three carbon atoms;
   R₄ is hydrogen, or a linear or branched alkyl group comprising between one and eight carbon atoms;
   R₁₂ is —OH or NH₂.
   L₁ is a linker unit, such that the linear dimension between the Cα carbon atoms connected by the linker unit is between about 6 and 15 Angstrom units;
   L₂ is a linker unit, such that the linear dimension between the Cα carbon atoms connected by the linker unit is between about 5 and 12 Angstrom units.

In one embodiment, the cyclic peptide is represented by Formula (I):

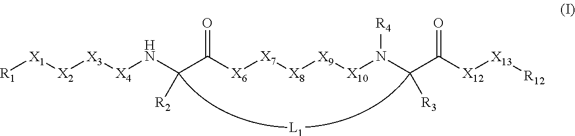

or a pharmaceutically acceptable salt thereof, wherein:
   X₁ is Thr, Asp, Phe, Glu, Asn, Gln, Ser, or Tyr;
   X₂ is Leu, His, Ser, Val, Trp, Ile, Phe, Tyr, or Thr;
   X₃ is Asp, Ser, Lys, Thr, Val, Asn, Leu, Glu, Ile, Gln, Gly, Pro, Ala, Trp, or Arg;
   X₄ is Asp, Trp, Gly, Ala, Pro, Tyr, Phe, or Glu;
   X₆ is Glu, Ser, Leu, Val, Trp, Gly, Ala, Arg, Tyr, Phe, Ile, Thr, Asp, or Lys;
   X₇ is Glu, Ala, Lys, Gly, Tyr, Phe, Arg, or Asp;
   X₈ is Met, Nleu, Gly, or Pro;
   X₉ is Asp or Glu;
   X₁₀ is Gly, Met, Nleu, Thr, or Ser;
   X₁₂ is Ser, Thr, Met, Leu, Ile, or Val;
   X₁₃ is Asp, Glu, or absent;
   R₁ is hydrogen, an acetyl group, or an acyl group;
   R₂ and R₃ are, independently, a hydrogen or an alkyl group comprising between one and three carbon atoms;
   R₄ is hydrogen, or a linear or branched alkyl group comprising between one and eight carbon atoms;
   R₁₂ is —OH or NH₂;
   L₁ is a linker unit, such that the linear dimension between the Cα carbon atoms connected by the linker unit is between about 6 and 15 Angstrom units.

In some embodiments, X₁ is Thr; X₂ is Leu, His, Ser, Val, or Trp; X₃ is Asp, or Ser; X₄ is Asp, Trp, or Gly; X₆ is Glu, Ser, Leu, Val, Trp, Gly, Ala, Arg, Tyr, or Phe; X₇ is Glu, Ala, Lys, or Gly; X₈ is Met; X₉ is Asp; X₁₀ is Gly, or Met; X₁₂ is Ser, or Thr; X₁₃ is Asp.

In one embodiment, the cyclic peptide is represented by Formula (II):

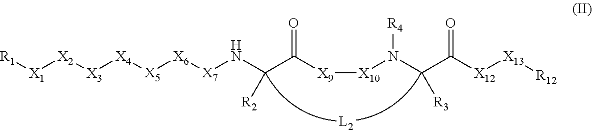

or a pharmaceutically acceptable salt thereof, wherein:
   X₁ is Thr, Asp, Phe, Glu, Asn, Gln, Ser, or Tyr;
   X₂ is Leu, His, Ser, Val, Trp, Ile, Phe, Tyr, or Thr;
   X₃ is Asp, Ser, Lys, Thr, Val, Asn, Leu, Glu, Ile, Gln, or Arg;
   X₄ is Asp, Trp, Gly, Ala, Pro, Tyr, Phe, or Glu;
   X₅ is Met or Gly;
   X₆ is Glu, Ser, Leu, Val, Trp, Gly, Ala, Arg, Tyr, Phe, Ile, Thr, Asp, or Lys;
   X₇ is Glu, Ala, Lys, Gly, Tyr, Phe, Arg, or Asp;
   X₉ is Asp or Glu;
   X₁₀ is Gly, Met, Nleu, Thr, or Ser;
   X₁₂ is Ser, Thr, Met, Leu, Ile, or Val;
   X₁₃ is Asp, Glu, or absent;
   R₁ is hydrogen, an acetyl group, or an acyl group;
   R₂ and R₃ are, independently, a hydrogen or an alkyl group comprising between one and three carbon atoms;
   R₄ is hydrogen or an alkyl group comprising between one and eight carbon atoms;
   R₁₂ is —OH or NH₂.
   L₂ is a linker unit, such that the linear dimension between the Cα carbon atoms connected by the linker unit is between about 5 and 12 Angstrom units.

In some embodiments, X₁ is Thr; X₂ is Leu; X₃ is Asp; X₄ is Asp; X₅ is Met; X₆ is Glu; X₇ is Glu; X₈ is Met; X₉ is Asp; X₁₀ is Gly; X₁₂ is Ser, Leu, or Val; X₁₃ is Asp.

Our studies revealed that structurally different linkers can be used to connect position 5 and position 11 of a cyclic peptide of formula (I) for the purpose of obtaining cyclic peptides that are able to bind to Hedgehog proteins and inhibit their signaling function. Our studies also revealed that structurally different linkers can be used to connect position 8 and position 11 of a cyclic peptide of formula (II) for the purpose of obtaining cyclic peptides that are able to bind to Hedgehog proteins and inhibit their signaling function

In some embodiments, the linker unit L₁ in the cyclic peptide of formula (I) and the linker unit L₂ in the cyclic peptide of formula (II) corresponds to —R₉—S—R₁₀— or —R₁₀—S—R₉—, wherein S is a sulfur atom, R₉ and R₁₀ are, independently, an alkyl, a substituted alkyl, an alkylaryl, or a substituted alkylaryl group. In some embodiments, the sulfur atom is oxidized to give rise to a sulfoxide (—S(O)—) or a sulfone group (—SO₂—). In embodiments, R₉ is an alkylaryl or a substituted alkylaryl group containing between 5 and 20 carbon atoms (C₅-C₂₀), whereas R₁₀ is an alkyl or a substituted alkyl group containing between 1 and 10 carbon atoms (C₁-C₁₀).

In embodiments, the linker unit L₁ in the cyclic peptide of formula (I) and the linker unit L₂ in the cyclic peptide of formula (II) are selected from the group consisting of

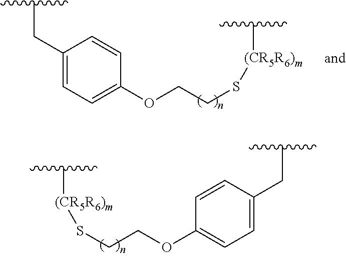

wherein n is an integer number comprised between 1 and 6; m is an integer number comprised between 1 and 3; R₅ and R₆ are, independently, a hydrogen atom or a methyl group.

In some embodiments, the linker unit L₁ in the cyclic peptide of formula (I) and the linker unit L₂ in the cyclic peptide of formula (II) corresponds to —(CH₂)ₙ—Z₁—(CH₂)ₚ—, wherein Z₁ is a functional group selected from the group consisting of —S—, —S—S—, —NHCO—, —CONH—, —CH═CH—, —(CH₂)₂—, —CH—CH—, and triazole; n and p are, independently, an integer number between 1 and 6.

In some embodiments, the linker unit L₁ corresponds to

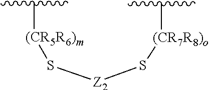

wherein Z₂ is a functional group selected from the group consisting of a C₁-C₁₀ alkyl or substituted alkyl, a C₂-C₁₀ alkenyl or substituted alkenyl, a C₆-C₁₂ aryl or substituted aryl, a C₈-C₁₄ alkylaryl or substituted alkylaryl group, —CH₂COCH₂—, and —CH₂COCOCH₂—; m and o are, independently, an integer number comprised between 1 and 3; R₅, R₆, R₇, and R₈ are, independently, a hydrogen atom or a methyl group.

In embodiments, the linker unit L₁ corresponds to

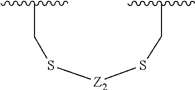

wherein Z₂ is a functional group selected from the group consisting of —(CH₂)ₙ—, wherein n is an integer number comprised between 1 and 4; —CH₂CH═CHCH₂—,

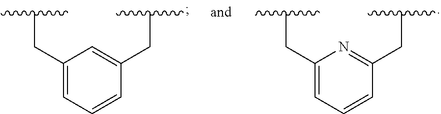 [SMILES: *Cc1cccc(C*)c1 ; *Cc1cccc(C*)n1]

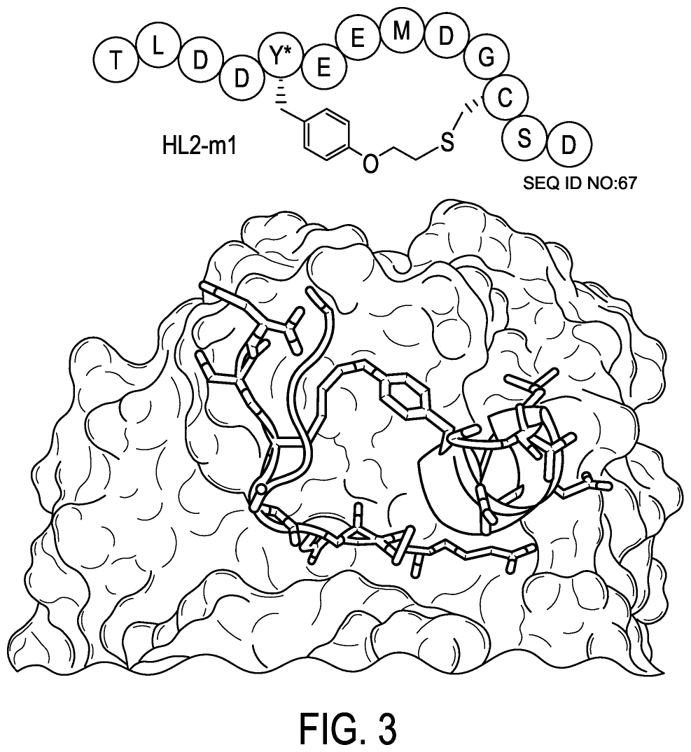

The cyclic peptides of formula (I) and (II) are presumed to bind to Hedgehog proteins by adopting a conformation that resembles that of the L2 loop in HHIP in the crystallized Shh/HHIP complex (FIG. 3). Accordingly, residue X₉ in a cyclic peptide of formula (I) or (II) is believed to mimic the role of Asp383 in HHIP, establishing an energetically important interaction with a protein-bound zinc ion within the HHIP L2 binding cleft of Sonic Hedgehog. In an embodiment, a residue X₉ in the cyclic peptides is an acidic amino acid (i.e., Asp, Glu) or another amino acid carrying a side-chain functional group capable of metal coordination such as His and Cys. Alternatively, a structural or functional isostere of these naturally occurring amino acids (i.e., Asp, Glu, His, Cys) can be introduced at this position to provide a cyclic peptide capable of inhibiting Hedgehog protein(s). In some embodiments, residue X₉ in the cyclic peptides of formula (I) and (II) is an aspartic acid, glutamic acid, histidine, or cysteine residue. In some embodiments, the amino acid residue X₉ in the cyclic peptide of formula (I) or (II) is an isostere of aspartic acid or an isostere of glutamic acid. In some embodiments, residue X₉ in the cyclic peptide of formula (I) and (II) is an amino acid analog of formula (III)

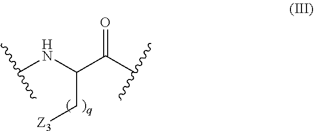

wherein q is 0, 1, or 2; and Z₃ is selected from the group consisting of —CONHOH, —N(OH)COCH₃, —CONHOCH₃, —ONHCOCH₃, —P(═O)(OH)₂, —P(═O)H(OH), —SO₃H, —SO₂NH₂, —NHSO₂CH₃, —CONHSO₂CH₃, —NHCONHSO₂CH₃, imidazole, 1,2,3-triazole, 1,2,4-triazole, tetrazole, —CH(OH)CF₃, —C(OH)₂CF₃, thiazolidine-2,4-dione, oxazolidine-2,4-dione, 1,2,4-oxadiazol-5(4H)-one, 1,2,4-thiadiazol-5(4H)-one, 3H-1,2,3,5-oxathiadiazole 2-oxide, 1,2,4-oxadiazole-5(4H)-thione, isoxazol-3-ol, isothiazol-3-ol, pyrrolidine-2,4-dione, furan-2,4(3H,5H)-dione, 3-hydroxycyclopent-2-enone, 3-hydroxycyclobut-3-ene-1,2-dione, and 2,6-difluorophenol.

Residues X₁, X₂, X₃, X₄, X₆, and X₇ in a cyclic peptide of formula (I) and residues X₁, X₂, X₃, X₄, X₅, X₆, and X₇ in a cyclic peptide of formula (II) are located within an alpha-helical region of these cyclic peptides when in complex with Sonic Hedgehog. The introduction of alpha-helix inducing residues within this region (e.g., Ala as X₇ residue in cyclic peptide of formula (I)) can stabilize the bioactive conformation of the cyclic peptide favoring binding to Hedgehog proteins. In certain embodiments, at least one of the amino acid residues X₁, X₂, X₃, X₄, X₆, and X₇ in the cyclic peptide of formula (I) and at least one of the amino acid residues X₁, X₂, X₃, X₄, X₅, X₆, and X₇ in the cyclic peptide of formula (II) is an alpha-helix inducing alpha amino acid. Art-known alpha-helix inducing alpha amino acids include, but are not restricted to, alpha-aminoisobutyric acid (Aib), 3,3,3-trifluoro-alanine, alpha,alpha-bis-trifluoromethyl-glycine, and 2-cyclopropylglycine, alpha,alpha-diethyl glycine (Deg), alpha,alpha-dipropyl glycine (Dpg), alpha,alpha-di-isobutyl glycine (Dibg), alpha,alpha-dihexyl glycine (Dhg), alpha,alpha-diphenyl-glycine (DΦg), alpha,alpha-dibenzyl glycine (Dbzg), alpha,alpha-cyclohexyl-glycine (Ac6c), and alpha,alpha-dihydroxymethyl glycine (Dmg). In some embodiments, at least one amino acid residue among residues X₁, X₂, X₃, X₄, X₆, and X₇ in the cyclic peptide of formula (I) and at least one amino acid residue among residues X₁, X₂, X₃, X₄, X₅, X₆, and X₇ in the cyclic peptide of formula (II) is an amino acid selected from the group consisting of alpha-aminoisobutyric acid (Aib), 3,3,3-trifluoro-alanine, alpha,alpha-bis-trifluoromethyl-glycine, 2-cyclopropylglycine, alpha,alpha-dihexyl glycine (Dhg), and alpha,alpha-cyclohexyl-glycine (Ac6c).

Residue X₁₀ in a cyclic peptide of formula (I) or (II) is located within a turn region of the molecule when bound to Sonic Hedgehog and that the introduction of amino acid analogs at this position can affect the conformation of the cyclic peptide, thereby favoring binding to Hedgehog proteins. Accordingly, in some embodiments, residue X₁₀ in the cyclic peptides of formula (I) and (II) is a D-amino acid, an N-substituted glycine residue, a beta amino acid, or a gamma amino acid. In embodiments, residue X₁₀ in the cyclic peptides of formula (I) and (II) is a D-amino acid selected from the group consisting of D-Met, D-Nleu, D-Thr, and D-Ser. In other embodiments, residue X₁₀ in the cyclic peptide of formula (I) and (II) is a N-substituted glycine residue corresponding to —N(R₉)CH₂C(O)—, wherein R₉ is selected from the group consisting of C₁-C₁₅ aliphatic, C₁-C₁₅ substituted aliphatic, C₅-C₁₅ aryl, C₅-C₁₅ substituted aryl, a C₆-C₁₅ alkylaryl, and C₆-C₁₅ substituted alkylaryl group.

We further discovered that the introduction of an aromatic residue in position X₄ of a cyclic peptide of formula (I) or (II) can be beneficial for favoring binding of these compounds to Hedgehog proteins, possibly by establishing interactions with a region of the proteins that is not contacted by the L2 loop of HHIP protein. Accordingly, in some embodiments, residue X₄ of the cyclic peptides of formula (I) and (II) is a non-naturally occurring alpha amino acid, whose side chain comprises an aryl group or a substituted aryl group. Representative examples of non-naturally occurring amino acids suitable for this purpose include, but are not limited to substituted tryptophan derivatives (e.g., 5-fluoro-tryptophan, 5-hydroxy-tryptophan, 5-methoxy-tryptophan, 5-methyl-tryptophan, 6-chloro-tryptophan, etc.), substituted phenylalanine derivatives (e.g., p-methoxy-Phe, p-acethyl-Phe, m,p,-difluoromethyl-Phe, pentafluoro-phenylalanine, o-trifluoromethyl-Phe, 3-chloro-Phe, m,p-dichloro-Phe, 0-phenyl-Phe, p-benzoyl-Phe, etc.), substituted tyrosine derivatives (e.g., 3-chloro-Tyr, 3-nitro-Tyr, 3,5-diiodo-Tyr, 3,3′,5-triiodo-thyronine, etc.), beta-(3-pyridyl)alanine, beta-(2-thienyl)alanine, beta-phenylalanine, beta-(1-naphtyl)alanine, beta-(2-naphtyl)alanine, 0-(1H-5-indolyl)alanine, 3-([1,1′-biphenyl]-4-yl)-2-aminopropanoic acid, 3-([2,2′-bipyridin]-5-yl)-2-aminopropanoic acid, and derivatives thereof. In some embodiments, residue X₄ in the cyclic peptide of formula (I) or (II) is an amino acid analog of formula (IV)

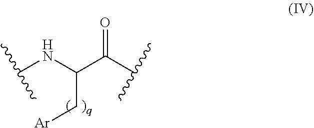

wherein q is 0, 1, or 2; and Ar is a C₅-C₁₅ aryl or a C₅-C₁₅ substituted aryl group.

In some embodiments, the carboxy terminal residue (i.e., the free carboxy group at the C-terminal end of the polypeptide) of the cyclic peptide of Formula (I) or (II) is modified (e.g., for protection against proteolytic degradation). In some embodiments, the modification is an amidation (replacement of the OH group by a NH₂ group).

In vertebrates, there are three Hedgehog homologues, namely Sonic (Shh), Indian (Ihh), and Desert Hedgehog (Dhh). The three Hedgehog homologs share a similar structure and have an overlapping albeit not identical biological function (Lees, Howie et al. 2005). Hedgehog is biosynthesized as a 45-kilodalton precursor protein, which undergoes a series of post-translational maturation events, including autoproteolysis, to give rise to the signaling molecules responsible for stimulation of the Hedgehog pathway in cells. As part of this maturation process, a cholesterol molecule is added to the C-terminus of the 20 kDa N-terminal signaling domain of Hedgehog proteins (typically referred to as Shh-N, Dhh-N, and Ihh-N). In addition, a palmitate molecule is added to the N-terminal amino group of Shh-N, Dhh-N, and Ihh-N by means of a N-palmitoyltransferase enzyme. This modification is required for efficient signaling, likely due to localization of these signaling proteins to the cell membrane where the Patched receptor is localized. The introduction of hydrophobic moiety (e.g., a fatty acid molecule) to the N-terminal or C-terminal end of the provided cyclic peptides can provide a means to localize these Hedgehog inhibitors to the cell membrane where the Patched receptor is located, thereby potentially potentiating the biological activity of these compounds. Most conveniently, the hydrophobic moiety such as a fatty acid molecule is added to the N-terminal end of the cyclic peptide.

In some embodiments, the cyclic peptide comprises a fatty acid or a fatty acid-like molecule. In some embodiments, the fatty acid or fatty acid-like molecule is attached directly to the N-terminal amino group of the X₁ residue, resulting in an N-terminal acyl group. In some embodiments, the fatty acid or fatty acid-like molecule is attached to an amine-containing spacer unit which is linked to the N-terminal amino group of the X₁ residue, resulting in acyl group connected to the N-terminal end of the cyclic peptide via a spacer unit. In some embodiments, the fatty acid or a fatty acid-like molecule is attached to the C-terminus of the cyclic peptide via a spacer unit. Representative examples of fatty acid molecules useful for this purpose include, but are not limited to, laurate, myristate, palmitate, stearate, arachidate, palmitoleate, oleate, linoleate, linolenate, and arachidonate. Representative examples of fatty acid-like molecules include linear and branched terminal alkanoic acid and terminal alkenoic acids comprising between 3 and 11 carbon atoms.

In some embodiments, the R₁ group in a cyclic peptide of formula (I) or (II) comprises a fatty acid molecule selected from the group consisting of C₃-C₂₀ alkanoic acids and C₄-C₂₀ alkenoic acids. In some embodiments, the fatty acid molecule contained within the R₁ group is connected to the N-terminus of the X₁ residue via a spacer unit. In some embodiments, the spacer unit connecting the fatty acid molecule to the X₁ residue is selected from the group consisting of (Xaa)ₙ, —NH(CH₂CH₂O)ₙC(O)—, and —NH(CH₂)ₙ(CH₂)ₚC(O)—, wherein Xaa is any amino acid, and n and p are, independently, an integer number between 1 and 6.

Beside their use in the context of targeting the Hedgehog pathway for therapeutic applications, the cyclic peptides disclosed here can be useful for the detection and/or imaging of Hedgehog proteins. Detection or imaging of Hedgehog proteins by means of the provided cyclic peptide can be carried out in a cell-free system, in a cell culture, in a tissue sample, and/or in an animal. For these applications, for example, a tag molecule, a label molecule, a polymer, a nanoparticle, and/or a solid support can be conjugated to the N-terminus, C-terminus, or to side chain of any of the amino acid residues comprised in the cyclic peptide, either directly or through a spacing unit. In an embodiment, this modification should not affect or only minimally affect the binding affinity of the cyclic peptide toward the Hedgehog protein(s). Most conveniently, such tag molecule, label molecule, polymer, nanoparticle, and solid support, among other molecules, is conjugated to the N-terminal end of the cyclic peptide, optionally via a spacer unit.

Representative examples of suitable spacer units include, but are not limited to, short (e.g., 2-15 amino acid-long) oligopeptide sequences (e.g., Gly-Gly, Gly-Ser, Gly-Gly-Gly, Gly-Ser-Gly-Ser (SEQ ID NO: 72), etc.), polyethylene glycol molecules (e.g., —NH(CH₂CH₂O)—C(O)ₙ—, wherein n is 1, 2, 3, 4, 5, 6, etc.), and alkyl spacers (e.g., —NH(CH₂)ₙ—C(O)—, wherein n is 1, 2, 3, 4, 5, 6, 8, 10, etc.).

In some embodiments, the R₁ group in the cyclic peptide of formula (I) or (II) comprises a tag molecule, a label molecule, an affinity label molecule, a photoaffinity label, a dye, a chromophore, a fluorescent molecule, a phosphorescent molecule, a chemiluminescent molecule, an energy transfer agent, a photocrosslinker molecule, a redox-active molecule, an isotopic label molecule, a spin label molecule, a metal chelator, a metal-comprising moiety, a heavy atom-comprising-moiety, a radioactive moiety, a contrast agent molecule, a MRI contrast agent, an isotopically labeled molecule, a PET agent, a photocaged moiety, a photoisomerizable moiety, a chemically cleavable group, a photocleavable group, an electron dense group, a magnetic group, an amino acid, a polypeptide, an antibody or antibody fragment, a carbohydrate, a monosaccharide, a polysaccharide, a nucleotide, a nucleoside, a DNA, a RNA, a siRNA, a polynucleotide, an antisense polynucleotide, a peptide nucleic acid (PNA), a fatty acid, a lipid, a cofactor, biotin, a biotin analogue, a biomaterial, a polymer, a water-soluble polymer, a polyethylene glycol derivative, a water-soluble dendrimer, a cyclodextrin, a small molecule, a protein-, nucleic acid-, or receptor-binding molecule, a biologically active molecule, a drug or drug candidate, a cytotoxic molecule, a solid support, a surface, a resin, a nanoparticle, or a quantum dot, or any combination thereof. In some embodiments, any of aforementioned molecules comprised within the R₁ group is linked to X₁ residue of the cyclic peptide of formula (I) or (II) via a spacer unit. In some embodiments, the spacer unit is selected from the group consisting of (Xaa)ₙ, —NH(CH₂CH₂O)ₙC(O)—, and —NH(CH₂)ₙ(CH₂)ₚC(O)—, wherein Xaa is any amino acid, and n and p are, independently, an integer number between 1 and 6.

The cyclic peptide of formula (I) or (II) may further comprise modifications that confer additional biological properties to these compounds such as enhanced resistance against proteolytic degradation, increased plasma half-life, cell permeability, increased or decreased binding to plasma proteins, etc. Such modifications include, for example, covalent attachment of fatty acids (e.g., C₆-C₁₈), conjugation to proteins such as albumin (see, for example, U.S. Pat. No. 7,268,113), PEGylation (see, for example, U.S. Pat. Nos. 7,585,837), and glycosylation.

A water-soluble polymer can be conjugated to the cyclic peptide of formula (I) or (II) for the purpose, for example, of increasing its stability in vivo in the context of the application of these compounds as pharmacological agents or as probes in animal studies. Such water-soluble polymers include, but are not limited to polyethylene glycol, polyethylene glycol propionaldehyde, mono C₁-C_{1O} alkoxy or aryloxy derivatives thereof monomethoxy-polyethylene glycol, polyvinyl pyrrolidone, polyvinyl alcohol, polyamino acids, divinylether maleic anhydride, N-(2-hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, including but not limited to methylcellulose and carboxymethyl cellulose, serum albumin, starch and starch derivatives, polypeptides, polyalkylene glycol and derivatives thereof, copolymers of polyalkylene glycols and derivatives thereof, polyvinyl ethyl ethers, and alpha-beta-poly[(2-hydroxyethyl)-aspartamide, and the like, or mixtures thereof.

In some embodiments, the R₁ group in the cyclic peptide of formula (I) or (II) comprises a water-soluble polymer. In other embodiments, the R₁ group comprises a water-soluble polymer that is linked to X₁ via a spacer unit. In some embodiments, the water-soluble polymer is selected from the group consisting of a functionalized or non-functionalized linear poly(ethyleneglycol) molecule, and a functionalized or non-functionalized branched poly(ethyleneglycol) molecule. In some embodiments, the R₁ group contains a polyether of formula —(CH₂—CH₂—O)ᵣ—, where r in an integer number between 10 and 1000. In some embodiments, the spacer unit is selected from the group consisting of (Xaa)ₙ, —NH(CH₂CH₂O),C(O)—, and —NH(CH₂)ₙ(CH₂)ₚC(O)—, wherein Xaa is any amino acid, and n and p are, independently, an integer number between 1 and 6.

The cyclic peptide provided herein can be attached to a solid support for the purpose of generating a device useful for detection or immobilization of at least one homolog of Hedgehog in a sample, e.g. for diagnostic applications, for enabling the screening and identification of other compounds capable of binding Hedgehog proteins, and/or for enabling the isolation or purification of Hedgehog proteins. Examples of solid supports that can be used include, but are not limited to, solid and semisolid matrixes, such as aerogels and hydrogels, resins, beads, biochips (including thin film coated biochips), microfluidic chip, a silicon chip, multi-well plates (also referred to as microtitre plates or microplates), membranes, cells, conducting and nonconducting metals, glass (including microscope slides) and magnetic supports. Other non-limiting examples of solid supports used in the methods and compositions described herein include silica gels, polymeric membranes, particles, derivatized plastic films, derivatized glass, derivatized silica, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like. In certain embodiments, the supports used in the methods and compositions described herein are supports used for surface analysis such as surface acoustic wave devices or devices utilizing evanescent wave analysis, such as surface plasmon resonance analysis. Other supports used in the methods and compositions described herein include, but are not limited to, resins used in peptide synthesis such as, by way of example only, polystyrene, PAM-resin, POLYHIPE™ resin, polyamide resin, polystyrene resin grafted with poly(ethylene glycol), polydimethyl-acrylamide resin and PEGA beads. The solid support can be, but is not limited to, in the form of a sheet, a multi-well plate, a bead or microbead, a slide, a microarray tray, and a test tube. Other suitable shapes and configurations for the solid support will also be recognized by the skilled artisan.

In some embodiments, the cyclic peptide is attached to a solid support. In some embodiments, the R₁ group in the compounds of formula (I) or (II) comprises a solid support. In other embodiments, the R₁ group in the compounds of formula (I) or (II) comprises a solid support that is linked to X₁ via a spacer unit. In some embodiments, the solid support is a resin. In some embodiments, the solid support is a chip. In some embodiments, the solid support is a surface. In some embodiments, the spacer unit is selected from the group consisting of (Xaa)ₙ, —NH(CH₂CH₂O)ₙC(O)—, and —NH(CH₂)ₙ(CH₂)ₚC(O)—, wherein Xaa is any amino acid, and n and p are, independently, an integer number between 1 and 6.

In some embodiments, the cyclic peptide provided herein comprises a warhead group. As used herein, the term “warhead” or “warhead group” refers to a functional group present on a compound provided herein wherein that functional group is capable of covalently binding to an amino acid residue (such as lysine, histidine, cysteine, or other residues capable of being covalently modified) in a protein, thereby forming an irreversible or reversible covalent complex with the protein. In some embodiments, the compound of formula (I) or (II) comprises a warhead group, wherein the warhead group is able to form a covalent bond with a lysine or a histidine residue. In some embodiments, the warhead group is a sulfonylfluoride group (—SO₂F). In some embodiments, the warhead sulfonylfluoride group is selected from the group consisting of 4-(sulfonylfluoride)benzoyl, 3-(sulfonylfluoride)benzoyl, 2-(sulfonylfluoride)benzoyl, benzenesulfonyl fluoride, naphthalenesulfonyl fluoride, phenylmethanesulfonyl fluoride, and —(CH₂)ₙSO₂F, where n is an integral number between 1 and 6. In some embodiments, the warhead is a (2-acetylphenyl)boronic acid group. In some embodiments, the warhead is a phenyl isothiocyanate (Ph-NCS) group. In some embodiments, the warhead is a vinylsulfonamido group (—NHSO₂CH═CH₂). In some embodiments, the warhead is —(CH₂)ᵣCH₂Br, where r is an integral number between 1 and 10. In some embodiments, the warhead group is attached to the side-chain of one of the amino acid residues of the cyclic peptide, either directly or via a spacing unit. In some embodiments, the warhead group is attached to N-terminus of the cyclic peptide, either directly or via a spacing unit. In some embodiments, the warhead group is attached to C-terminus of the cyclic peptide, either directly or via a spacing unit.

In some embodiments, the configuration of the alpha carbon in the amino acids and amino acid analogs comprised in the cyclic peptides of formula (I) or (II) is S. In other embodiments, the configuration of the alpha carbon in the amino acids and amino acid analogs comprised in the cyclic peptides of formula (I) or (II) is R. In some embodiments, some of the amino acids and amino acid analogs comprised in a cyclic peptides of formula (I) or (II) have an alpha carbon atom in S configuration, whereas some of the amino acids and amino acid analogs have an alpha carbon atom in R configuration.

In some embodiments, at least one of the amino acids and/or amino acid analogs comprised in the cyclic peptide is an alpha,alpha-disubstituted amino acid. Examples of alpha,alpha-disubstituted amino acids that can be incorporated in a cyclic peptide provided herein include, but are not limited to, alpha-methyl-(S)-cysteine, alpha-methyl-(R)-cysteine, alpha-aminoisobutyric acid (Aib), alpha,alpha-bis-trifluoromethyl-glycine, 2-cyclopropylglycine, alpha,alpha-dihexyl glycine, and alpha,alpha-cyclohexyl-glycine.

Alkylation of the backbone amide group (e.g., N-methylation) of at least one amino acid residue in a peptide molecule can potentially confer this molecule with desirable properties such as, for example, increased proteolytic stability and/or cell permeability (Chatterjee, Gilon et al. 2008). In some embodiments, at least one of the amino acids or amino acid analogs comprised in the cyclic peptide is an N-alkylated amino acid. In some embodiments, at least one of the amino acids or amino acid analogs comprised in the cyclic peptide is an N-methylated amino acid. Examples of N-alkylated amino acids include, but are not limited to such as, for example, N-methyl-(S)-cysteine, N-methyl-(R)-cysteine, N-methyl-methionine, N-butyl-glycine.

In some embodiments, the cyclic peptide inhibitors comprise analogs of a compound of formula (I) or (II) as described above, in which at least one amino acid residue has been substituted for a functionally equivalent amino acid residue or a functionally equivalent isostere of said amino acid residue. For example, at least one amino acid residue within the cyclic peptide can be substituted by another naturally occurring or non-naturally occurring amino acid of a similar polarity (having similar physico-chemical properties) which acts as a functional equivalent of said amino acid residue(s). For example, a positively charged (basic) amino acid such as arginine, lysine, and histidine can be substituted for another naturally occurring basic amino acid (i.e., arginine, lysine, or histidine) as well as a non-naturally occurring basic amino acid such as, for example, homoarginine, ornithine, 2,4-diaminobutyric acid, and 2,3-diaminopropionic acid. A nonpolar (hydrophobic) amino acid can be substituted for a naturally occurring nonpolar amino acid such as leucine, isoleucine, alanine, valine, proline, and methionine as well as for a non-naturally occurring nonpolar amino acid such as, for example, norleucine, norvaline, 2-ethylglycine, and the like. An aromatic amino acid can be substituted for a naturally occurring aromatic amino acid such as phenylalanine, tyrosine, tryptophan, histidine as well as for a non-naturally occurring aromatic amino acid such as, for example, any of phenylalanine, tyrosine, and tryptophan derivatives described earlier. A polar amino acid can be substituted for a naturally occurring polar amino acid such as serine, threonine, asparagine and glutamine as well as for a non-naturally occurring polar amino acid such as, for example, A cysteine residue can be substituted for another thiol-containing amino acid such as, for example, homocysteine, 5-mercaptonorvaline, and penicillamine. In some embodiments, this cysteine residue or analog thereof (e.g., homocysteine, 5-mercaptonorvaline, and penicillamine) is part of the inter-side-chain linkage of the cyclic peptide. Examples of structural or functional isosteres of a natural amino acid residue such as an aspartic or a glutamic acid residue have been described earlier.

One aspect of the present invention is the discovery that cyclic peptides comprising part of the HHIP L2 sequence can provide effective binders for the Hedgehog proteins and can inhibit Hedgehog protein-mediated activation of Hedgehog pathway in a cell. It is understood that, based on this information, analogs of the compounds of formula (I) or (II) can be further generated by various means for the purpose of developing a functionally equivalent or an improved inhibitor of the Hedgehog proteins. These means include, but are not limited to substituting at least one amino acid residue with the amino acid sequence comprised by a compound of formula (I) or (II) with another amino acid, amino acid analog, or amino acid isostere, or by varying the linker unit L₁ in a compound of formula (I) and the linker unit L₂ in a compound of formula (II). As described further below, an informed decision concerning which amino acid can be substituted and with which amino acid, amino acid analog, or amino acid isostere, as well as concerning which linker unit to use, can be made by measuring the effect of the substitution on the Hedgehog protein inhibitory activity of the compound as determined by a cell-free or cell-based functional assay.

All peptide formulas or sequences shown herein are written from left to right in the direction from the amino-terminus (N-terminus) to the carboxy-terminus (C-terminus). The abbreviations used herein for both naturally occurring amino acids and selected non-naturally occurring amino acids are reproduced in the following Table:

| | Abbreviation | |
|---|---|---|
| Amino Acid | 3-letter | 1-letter |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Glv | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Ornithine | Orn | — |
| 2,4-diaminobutyric acid | Dab | — |
| 2,3-diaminopropionic acid | Dap | — |
| Norleucine | Nleu | — |
| Norvaline | Nval | — |
| 2-Aminobutyric acid | Abu | — |
| 2-Aminoisobutyric acid | Aib | — |
| Penicillamine | Pen | — |
| Any amino acid | Xaa | — |

In some embodiments, the cyclic peptide provided herein is a compound of formula (I) that comprises an amino acid sequence which is at least about 40%, 50%, 60%, 80%, 90%, or 95% identical to the peptide sequence corresponding to SEQ ID NO: 1.

In other embodiments, the cyclic peptide provided herein is a compound of formula (II) that comprises an amino acid sequence which is at least about 40%, 50%, 60%, 80%, 90%, or 95% identical to the peptide sequence corresponding to SEQ ID NO: 1.

The structures of exemplary cyclic peptide Hedgehog protein inhibitors are shown in Table 1 below.

TABLE 1

| | AMINO ACID SEQUENCE | | | | | | | | | | | | | | |
|---|---|---|---|---|---|---|---|---|---|---|---|---|---|---|---|
| Name | R₁ | X₁ | X₂ | X₃ | X₄ | X₅ | X₆ | X₇ | X₈ | X₉ | X₁₀ | X₁₁ | X₁₂ | X₁₃ | Linker |
| FLAG-HL2-m1 | FLAG | Thr | Leu | Asp | Asp | X | Glu | Glu | Met | Asp | Gly | X | Ser | Asp-OH | L-1 |
| SEQ ID NO: 7 | | | | | | | | | | | | | | | |
| HL2-m1 | Ac | Thr | Leu | Asp | Asp | X | Glu | Glu | Met | Asp | Gly | X | Ser | Asp-NH₂ | L-1 |
| SEQ ID NO: 7 | | | | | | | | | | | | | | | |
| FLAG-HL2-m1r | FLAG | Thr | Leu | Asp | Asp | X | Glu | Glu | Met | Asp | Gly | X | Ser | Asp-OH | L-2 |
| SEQ ID NO: 7 | | | | | | | | | | | | | | | |
| FLAG-HL2-m1.1 | FLAG | Thr | Leu | Trp | Asp | X | Glu | Glu | Met | Asp | Gly | X | Ser | Asp-OH | L-1 |
| SEQ ID NO: 8 | | | | | | | | | | | | | | | |
| FLAG-HL2-m1.2 | FLAG | Thr | Leu | Gly | Asp | X | Glu | Glu | Met | Asp | Gly | X | Ser | Asp-OH | L-1 |
| SEQ ID NO: 9 | | | | | | | | | | | | | | | |
| FLAG-HL2-m1.3 | FLAG | Thr | Leu | Ala | Asp | X | Glu | Glu | Met | Asp | Gly | X | Ser | Asp-OH | L-1 |
| SEQ ID NO: 10 | | | | | | | | | | | | | | | |
| FLAG-HL2-m1.4 | FLAG | Thr | Leu | Pro | Asp | X | Glu | Glu | Met | Asp | Gly | X | Ser | Asp-OH | L-1 |
| SEQ ID NO: 11 | | | | | | | | | | | | | | | |
| FLAG-HL2-m1.5 | FLAG | Thr | Leu | Asp | Asp | X | Ser | Glu | Met | Asp | Gly | X | Ser | Asp-OH | L-1 |
| SEQ ID NO: 12 | | | | | | | | | | | | | | | |
| FLAG-HL2-m1.6 | FLAG | Thr | Leu | Asp | Asp | X | Leu | Glu | Met | Asp | Gly | X | Ser | Asp-OH | L-1 |
| SEQ ID NO: 13 | | | | | | | | | | | | | | | |
| FLAG-HL2-m1.7 | FLAG | Thr | Leu | Asp | Asp | X | Val | Glu | Met | Asp | Gly | X | Ser | Asp-OH | L-1 |
| SEQ ID NO: 14 | | | | | | | | | | | | | | | |
| FLAG-HL2-m1.8 | FLAG | Thr | Leu | Asp | Asp | X | Trp | Glu | Met | Asp | Gly | X | Ser | Asp-OH | L-1 |
| SEQ ID NO: 15 | | | | | | | | | | | | | | | |
| FLAG-HL2-m1.9 | FLAG | Thr | Leu | Asp | Asp | X | Glu | Lys | Met | Asp | Gly | X | Ser | Asp-OH | L-1 |
| SEQ ID NO: 16 | | | | | | | | | | | | | | | |
| FLAG-HL2-m1.10 | FLAG | Thr | Leu | Asp | Asp | X | Glu | Ala | Met | Asp | Gly | X | Ser | Asp-OH | L-1 |
| SEQ ID NO: 17 | | | | | | | | | | | | | | | |
| FLAG-HL2-m1.11 | FLAG | Thr | Leu | Asp | Asp | X | Glu | Gly | Met | Asp | Gly | X | Ser | Asp-OH | L-1 |
| SEQ ID NO: 18 | | | | | | | | | | | | | | | |
| FLAG-HL2-m1.12 | FLAG | Thr | Leu | Asp | Asp | X | Glu | Tyr | Met | Asp | Gly | X | Ser | Asp-OH | L-1 |
| SEQ ID NO: 19 | | | | | | | | | | | | | | | |
| FLAG-HL2-m1.13 | FLAG | Thr | Leu | Asp | Asp | X | Glu | Glu | Met | Asp | Thr | X | Ser | Asp-OH | L-1 |
| SEQ ID NO: 20 | | | | | | | | | | | | | | | |
| FLAG-HL2-m1.14 | FLAG | Thr | Leu | Asp | Asp | X | Glu | Glu | Met | Asp | Met | X | Ser | Asp-OH | L-1 |
| SEQ ID NO: 21 | | | | | | | | | | | | | | | |
| FLAG-HL2-m1.15 | FLAG | Thr | Leu | Asp | Asp | X | Glu | Glu | Met | Asp | Gly | X | Thr | Asp-OH | L-1 |
| SEQ ID NO: 22 | | | | | | | | | | | | | | | |
| FLAG-HL2-m1.16 | FLAG | Thr | Leu | Asp | Asp | X | Glu | Glu | Met | Asp | Gly | X | Leu | Asp-OH | L-1 |
| SEQ ID NO: 23 | | | | | | | | | | | | | | | |
| FLAG-HL2-m2 | FLAG | Thr | Leu | Asp | Asp | X | Glu | Glu | Met | Asp | Gly | X | Met | Asp-OH | L-1 |
| SEQ ID NO: 24 | | | | | | | | | | | | | | | |
| HL2-m2 | Ac | Thr | Leu | Asp | Asp | X | Glu | Glu | Met | Asp | Gly | X | Met | Asp-NH₂ | L-1 |
| SEQ ID NO: 24 | | | | | | | | | | | | | | | |
| FLAG-HL2-m2.1 | FLAG | Thr | Leu | Asp | Asp | X | Val | Glu | Met | Asp | Gly | X | Ser | Asp-OH | L-1 |
| SEQ ID NO: 25 | | | | | | | | | | | | | | | |
| FLAG-HL2-m2.2 | FLAG | Thr | Leu | Asp | Asp | X | Gly | Glu | Met | Asp | Gly | X | Leu | Asp-OH | L-1 |
| SEQ ID NO: 26 | | | | | | | | | | | | | | | |
| FLAG-HL2-m2.3 | FLAG | Thr | Leu | Asp | Asp | X | Ala | Glu | Met | Asp | Gly | X | Met | Asp-OH | L-1 |
| SEQ ID NO: 27 | | | | | | | | | | | | | | | |
| FLAG-HL2-m2.4 | FLAG | Thr | Leu | Asp | Asp | X | Val | Glu | Met | Asp | Gly | X | Leu | Asp-OH | L-1 |
| SEQ ID NO: 28 | | | | | | | | | | | | | | | |
| FLAG-HL2-m2.5 | FLAG | Thr | Leu | Asp | Asp | X | Glu | Lys | Met | Asp | Met | X | Met | Asp-OH | L-1 |
| SEQ ID NO: 29 | | | | | | | | | | | | | | | |
| FLAG-HL2-m2.6 | FLAG | Thr | Leu | Asp | Asp | X | Glu | Tyr | Met | Asp | Met | X | Met | Asp-OH | L-1 |
| SEQ ID NO: 30 | | | | | | | | | | | | | | | |
| FLAG-HL2-m2.7 | FLAG | Thr | Leu | Asp | Trp | X | Glu | Glu | Met | Asp | Thr | X | Leu | Asp-OH | L-1 |
| SEQ ID NO: 31 | | | | | | | | | | | | | | | |
| FLAG-HL2-m2.8 | FLAG | Thr | Leu | Asp | Gly | X | Glu | Glu | Met | Asp | Met | X | Met | Asp-OH | L-1 |
| SEQ ID NO: 32 | | | | | | | | | | | | | | | |
| FLAG-HL2-m2.9 | FLAG | Thr | Leu | Asp | Trp | X | Glu | Glu | Met | Asp | Thr | X | Met | Asp-OH | L-1 |
| SEQ ID NO: 33 | | | | | | | | | | | | | | | |
| FLAG-HL2-m3 | FLAG | Thr | Leu | Asp | Trp | X | Glu | Glu | Met | Asp | Met | X | Thr | Asp-OH | L-1 |
| SEQ ID NO: 34 | | | | | | | | | | | | | | | |
| HL2-m3 | Ac | Thr | Leu | Asp | Trp | X | Glu | Glu | Met | Asp | Met | X | Thr | Asp-NH₂ | L-1 |
| SEQ ID NO: 34 | | | | | | | | | | | | | | | |
| FLAG-HL2-m3.1 | FLAG | Thr | Leu | Ser | Trp | X | Glu | Glu | Met | Asp | Met | X | Thr | Asp-OH | L-1 |
| SEQ ID NO: 35 | | | | | | | | | | | | | | | |
| FLAG-HL2-m4 | FLAG | Thr | Leu | Asp | Trp | X | Glu | Ala | Met | Asp | Met | X | Thr | Asp-OH | L-1 |
| SEQ ID NO: 36 | | | | | | | | | | | | | | | |
| FLAG-HL2-m5 | FLAG | Thr | Leu | Ser | Trp | X | Glu | Ala | Met | Asp | Met | X | Thr | Asp-OH | L-1 |
| SEQ ID NO: 37 | | | | | | | | | | | | | | | |
| HL2-m5 | Ac | Thr | Leu | Ser | Trp | X | Glu | Ala | Met | Asp | Met | X | Thr | Asp-NH₂ | L-1 |
| SEQ ID NO: 37 | | | | | | | | | | | | | | | |
| FLAG-HL2-m5.1 | FLAG | Thr | Leu | Ser | Trp | X | Leu | Ala | Met | Asp | Met | X | Thr | Asp-OH | L-1 |
| SEQ ID NO: 38 | | | | | | | | | | | | | | | |
| FLAG-HL2-m5.2 | FLAG | Thr | Leu | Ser | Trp | X | Phe | Ala | Met | Asp | Met | X | Thr | Asp-OH | L-1 |
| SEQ ID NO: 39 | | | | | | | | | | | | | | | |
| FLAG-HL2-m5.3 | FLAG | Thr | Leu | Ser | Trp | X | Tyr | Ala | Met | Asp | Met | X | Thr | Asp-OH | L-1 |
| SEQ ID NO: 40 | | | | | | | | | | | | | | | |
| FLAG-HL2-m5.4 | FLAG | Thr | Leu | Ser | Trp | X | Arg | Ala | Met | Asp | Met | X | Thr | Asp-OH | L-1 |
| SEQ ID NO: 41 | | | | | | | | | | | | | | | |
| FLAG-HL2-m5.5 | FLAG | Thr | His | Ser | Trp | X | Glu | Ala | Met | Asp | Met | X | Thr | Asp-OH | L-1 |
| SEQ ID NO: 42 | | | | | | | | | | | | | | | |
| FLAG-HL2-m5.6 | FLAG | Thr | Ser | Ser | Trp | X | Glu | Ala | Met | Asp | Met | X | Thr | Asp-OH | L-1 |
| SEQ ID NO: 43 | | | | | | | | | | | | | | | |
| FLAG-HL2-m5.7 | FLAG | Thr | Val | Ser | Trp | X | Glu | Ala | Met | Asp | Met | X | Thr | Asp-OH | L-1 |
| SEQ ID NO: 44 | | | | | | | | | | | | | | | |
| FLAG-HL2-m5.8 | FLAG | Thr | Trp | Ser | Trp | X | Glu | Ala | Met | Asp | Met | X | Thr | Asp-OH | L-1 |
| SEQ ID NO: 45 | | | | | | | | | | | | | | | |
| FLAG-HL2-m5.9 | FLAG | Asp | Leu | Ser | Trp | X | Glu | Ala | Met | Asp | Met | X | Thr | Asp-OH | L-1 |
| SEQ ID NO: 46 | | | | | | | | | | | | | | | |
| FLAG-HL2-m5.10 | FLAG | Phe | Leu | Ser | Trp | X | Glu | Ala | Met | Asp | Met | X | Thr | Asp-OH | L-1 |
| SEQ ID NO: 47 | | | | | | | | | | | | | | | |
| FLAG-HL2-m5.11 | FLAG | Glu | Leu | Ser | Trp | X | Glu | Ala | Met | Asp | Met | X | Thr | Asp-OH | L-1 |
| SEQ ID NO: 48 | | | | | | | | | | | | | | | |
| FLAG-HL2-m5.12 | FLAG | Asn | Leu | Ser | Trp | X | Glu | Ala | Met | Asp | Met | X | Thr | Asp-OH | L-1 |
| SEQ ID NO: 49 | | | | | | | | | | | | | | | |
| FLAG-HL2-m5 | FLAG | Gln | Leu | Ser | Trp | X | Glu | Ala | Met | Asp | Met | X | Thr | Asp-OH | L-1 |
| SEQ ID NO: 50 | | | | | | | | | | | | | | | |

(X = amino acids linked via the linker unit)

Wherein X is the linking point between the amino acids and the linker; “Ac” corresponds to an acetyl group; “FLAG” corresponds to the oligopeptide sequence MDYKDDDDKGSGS- (SEQ ID NO: 51), i.e., a FLAG tag sequence followed by a -(GS)₂- spacer unit;
L-1 corresponds to

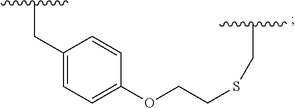 [SMILES: *CSCCOc1ccc(C*)cc1]

L-2 corresponds to O

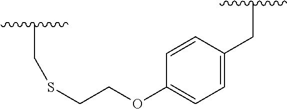 [SMILES: *CSCCOc1ccc(C*)cc1]

Xaa-OH=amino acid with free C-terminus (free carboxylic group); Xaa-NH₂=amino acid with amidated C-terminus.

Other exemplary cyclic peptide Hedgehog protein inhibitors are shown in Table 2 below.

TABLE 2

| | AMINO ACID SEQUENCE | | | | | | | | | | | | | | |
|---|---|---|---|---|---|---|---|---|---|---|---|---|---|---|---|
| Name | R₁ | X₁ | X₂ | X₃ | X₄ | X₅ | X₆ | X₇ | X₈ | X₉ | X₁₀ | X₁₁ | X₁₂ | X₁₃ | Linker |
| m5-s1 | Ac | Thr | Leu | Ser | Trp | X | Glu | Ala | Met | Asp | Met | X | Thr | Asp-NH₂ | L-3 |
| SEQ ID NO: 5 | | | | | | | | | | | | | | | |
| m5-s2 | Ac | Thr | Leu | Ser | Trp | X | Glu | Ala | Met | Asp | Met | X | Thr | Asp-NH₂ | L-4 |
| SEQ ID NO: 5 | | | | | | | | | | | | | | | |
| m5-s3 | Ac | Thr | Leu | Ser | Trp | X | Glu | Ala | Met | Asp | Met | X | Thr | Asp-NH₂ | L-5 |
| SEQ ID NO: 5 | | | | | | | | | | | | | | | |
| m5-s4 | Ac | Thr | Leu | Ser | Trp | X | Glu | Ala | Met | Asp | Met | X | Thr | Asp-NH₂ | L-6 |
| SEQ ID NO: 5 | | | | | | | | | | | | | | | |
| m5-s5 | Ac | Thr | Leu | Ser | Trp | X | Glu | Ala | Met | Asp | Met | X | Thr | Asp-NH₂ | L-7 |
| SEQ ID NO: 5 | | | | | | | | | | | | | | | |
| m5-s6 | Ac | Thr | Leu | Ser | Trp | X | Glu | Ala | Met | Asp | Met | X | Thr | Asp-NH₂ | L-8 |
| SEQ ID NO: 5 | | | | | | | | | | | | | | | |
| m5-s7 | Ac | Thr | Leu | Ser | Trp | X | Glu | Ala | Met | Asp | Met | X | Thr | Asp-NH₂ | L-9 |
| SEQ ID NO: 5 | | | | | | | | | | | | | | | |
| m5-s8 | Ac | Thr | Leu | Ser | Trp | X | Glu | Ala | Met | Asp | Met | X | Thr | Asp-NH₂ | L-10 |
| SEQ ID NO: 5 | | | | | | | | | | | | | | | |
| m5-s9 | Ac | Thr | Leu | Ser | Trp | X | Glu | Ala | Met | Asp | Met | X | Thr | Asp-NH₂ | L-11 |
| SEQ ID NO: 5 | | | | | | | | | | | | | | | |
| m5-s10 | Ac | Thr | Leu | Ser | Trp | X | Glu | Ala | Met | Asp | Met | X | Thr | Asp-NH₂ | L-12 |
| SEQ ID NO: 5 | | | | | | | | | | | | | | | |
| m5-s11 | Ac | Thr | Leu | Ser | Trp | X | Glu | Ala | Met | Asp | Met | X | Thr | Asp-NH₂ | L-13 |
| SEQ ID NO: 5 | | | | | | | | | | | | | | | |
| Biot-HL2-m5 | Biot-(GS)₂ | Thr | Leu | Ser | Trp | X | Glu | Ala | Met | Asp | Met | X | Thr | Asp-NH₂ | L-1 |
| SEQ ID NO: 5 | | | | | | | | | | | | | | | |
| Biot-βA-HL2-m5 | Biot-βAla | Thr | Leu | Ser | Trp | X | Glu | Ala | Met | Asp | Met | X | Thr | Asp-NH₂ | L-1 |
| SEQ ID NO: 5 | | | | | | | | | | | | | | | |
| Biot-m5-s1 | Biot-(GS)₂ | Thr | Leu | Ser | Trp | X | Glu | Ala | Met | Asp | Met | X | Thr | Asp-NH₂ | L-3 |
| SEQ ID NO: 5 | | | | | | | | | | | | | | | |
| Biot-m5-s2 | Biot-(GS)₂ | Thr | Leu | Ser | Trp | X | Glu | Ala | Met | Asp | Met | X | Thr | Asp-NH₂ | L-4 |
| SEQ ID NO: 5 | | | | | | | | | | | | | | | |
| Biot-m5-s3 | Biot-(GS)₂ | Thr | Leu | Ser | Trp | X | Glu | Ala | Met | Asp | Met | X | Thr | Asp-NH₂ | L-5 |
| SEQ ID NO: 5 | | | | | | | | | | | | | | | |
| Biot-m5-s4 | Biot-(GS)₂ | Thr | Leu | Ser | Trp | X | Glu | Ala | Met | Asp | Met | X | Thr | Asp-NH₂ | L-6 |
| SEQ ID NO: 5 | | | | | | | | | | | | | | | |
| Biot-m5-s7 | Biot-(GS)₂ | Thr | Leu | Ser | Trp | X | Glu | Ala | Met | Asp | Met | X | Thr | Asp-NH₂ | L-9 |
| SEQ ID NO: 5 | | | | | | | | | | | | | | | |
| Biot-m5-s8 | Biot-(GS)₂ | Thr | Leu | Ser | Trp | X | Glu | Ala | Met | Asp | Met | X | Thr | Asp-NH₂ | L-10 |
| SEQ ID NO: 5 | | | | | | | | | | | | | | | |
| Biot-m5-s9 | Biot-(GS)₂ | Thr | Leu | Ser | Trp | X | Glu | Ala | Met | Asp | Met | X | Thr | Asp-NH₂ | L-11 |
| SEQ ID NO: 5 | | | | | | | | | | | | | | | |
| Biot-m5-s10 | Biot-(GS)₂ | Thr | Leu | Ser | Trp | X | Glu | Ala | Met | Asp | Met | X | Thr | Asp-NH₂ | L-12 |
| SEQ ID NO: 5 | | | | | | | | | | | | | | | |
| Biot-m5-s7.2 | Biot-(GS)₂ | Thr | Leu | Ser | Trp | X | Glu | Aib | Met | Asp | Met | X | Thr | Asp-NH₂ | L-9 |
| SEQ ID NO: 52 | | | | | | | | | | | | | | | |
| Biot-m5-s7.2 | Biot-(GS)₂ | Thr | Leu | Ser | Trp | X | Glu | Ala | Met | Asp | Nleu | X | Thr | Asp-NH₂ | L-9 |
| SEQ ID NO: 53 | | | | | | | | | | | | | | | |
| Biot-m5-s7.3 | Biot-(GS)₂ | Thr | Leu | Ser | Trp | X | Glu | Ala | Met | Asp | N(n-butyl) | X | Thr | Asp-NH₂ | L-9 |
| SEQ ID NO: 54 | | | | | | | | | | | Gly | | | | |
| Biot-m5-s7.4 | Biot-(GS)₂ | Thr | Leu | Ser | Trp | X | Glu | Ala | Met | Asp | Met | ^{D}X | Thr | Asp-NH₂ | L-9 |
| SEQ ID NO: 55 | | | | | | | | | | | | | | | |
| Biot-m5-s7.5 | Biot-(GS)₂ | Thr | Leu | Ser | Trp | X | Glu | Ala | Met | Asp | Met | X | Thr | Asp-NH₂ | L-9b |
| SEQ ID NO: 5 | | | | | | | | | | | | | | | |

(X = amino acids linked via the linker unit)

Wherein X is the linking point between the amino acids and the linker; “Ac”=acetyl group; “Biot”=biotin; “(GS)₂”=-Gly-Ser-Gly-Ser- (SEQ ID NO: 72); “βAla”=beta-alanine; Aib=2-aminoisobutyric acid; Nleu=norleucine; N(n-butyl)Gly=N-(n-butyl)-glycine; ^{D}X=D-configured amino acid residue; Xaa-NH₂=amino acid with amidated C-terminus; L-3=—CH₂SSCH₂—; L-4=—CH₂SCH₂SCH₂—; L-5=—CH₂S(CH₂)₂SCH₂—; L-6=—CH₂S(CH₂)₃SCH₂—; L-7=—CH₂SCH₂C(O)CH₂SCH₂—; L-8=—CH₂SCH₂C(O)C(O)CH₂SCH₂—; L-9=—CH₂SCH₂CH═CHCH₂SCH₂—; L-10=

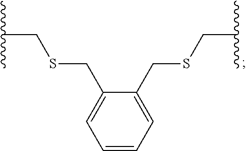 [SMILES: *CSCc1ccccc1CSC*]

L-11 corresponds to

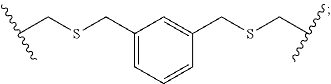 [SMILES: *CSCc1cccc(CSC*)c1]

L-12 corresponds to

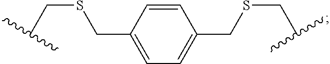 [SMILES: *CSCc1ccc(CSC*)cc1]

L-13 corresponds to

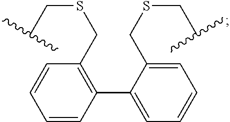 [SMILES: *CSCc1ccccc1-c1ccccc1CSC*]

L-9b corresponds to L-9=—CH₂SCH₂CH═CHCH₂SCH₂CH₂—.

Other exemplary cyclic peptide Hedgehog protein inhibitors are shown in Table 3 below.

TABLE 3

| | AMINO ACID SEQUENCE | | | | | | | | | | | | | | |
|---|---|---|---|---|---|---|---|---|---|---|---|---|---|---|---|
| Name | R₁ | X₁ | X₂ | X₃ | X₄ | X₅ | X₆ | X₇ | X₈ | X₉ | X₁₀ | X₁₁ | X₁₂ | X₁₃ | Linker |
| FLAG-HL2-2m1 | FLAG | Thr | Leu | Asp | Asp | Met | Glu | Glu | X | Asp | Gly | X | Ser | Asp-OH | L-1 |
| SEQ ID NO: 56 | | | | | | | | | | | | | | | |
| FLAG-HL2-2m1.1 | FLAG | Thr | Val | Ser | Asp | Gly | Glu | Glu | X | Asp | Gly | X | Leu | Asp-OH | L-1 |
| SEQ ID NO: 57 | | | | | | | | | | | | | | | |
| FLAG-HL2-2m1.2 | FLAG | Thr | Arg | Gly | Asp | Gly | Glu | Glu | X | Asp | Gly | X | Val | Asp-OH | L-1 |
| SEQ ID NO: 58 | | | | | | | | | | | | | | | |
| FIL2-2m1.1 | Ac | Thr | Val | Ser | Asp | Gly | Glu | Glu | X | Asp | Gly | X | Leu | Asp-NH₂ | L-1 |
| SEQ ID NO: 57 | | | | | | | | | | | | | | | |
| FIL2-2m1.2 | Ac | Thr | Arg | Gly | Asp | Gly | Glu | Glu | X | Asp | Gly | X | Val | Asp-NH₂ | L-1 |
| SEQ ID NO: 58 | | | | | | | | | | | | | | | |
| 2m1.1s1 | Ac | Thr | Val | Ser | Asp | Gly | Glu | Glu | X | Asp | Gly | X | Leu | Asp-NH₂ | L-9 |
| SEQ ID NO: 59 | | | | | | | | | | | | | | | |
| 2m1.2s1 | Ac | Thr | Arg | Gly | Asp | Gly | Glu | Glu | X | Asp | Gly | X | Val | Asp-NH₂ | L-9 |
| SEQ ID NO: 58 | | | | | | | | | | | | | | | |

(X = amino acids linked via the linker unit)

Wherein X is the linking point between the amino acids and the linker; “Ac”=acetyl group, “FLAG”=MDYKDDDDKGSGS- (SEQ ID NO: 51), Xaa-NH₂=amino acid with amidated C-terminus; Xaa-OH=amino acid with free C-terminal carboxylic group; L-1 corresponds to

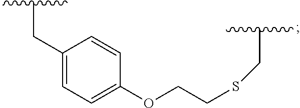 [SMILES: *CSCCOc1ccc(C*)cc1]

L-9=—CH₂SCH₂CH═CHCH₂SCH₂—.

Other exemplary cyclic peptide Hedgehog protein inhibitors are shown in Table 4 below.

TABLE 4

| | AMINO ACID SEQUENCE | | | | | | | | | | | | | | |
|---|---|---|---|---|---|---|---|---|---|---|---|---|---|---|---|
| Name | R₁ | X₁ | X₂ | X₃ | X₄ | X₅ | X₆ | X₇ | X₈ | X₉ | X₁₀ | X₁₁ | X₁₂ | X₁₃ | Linker |
| m5-s4.1 | Ac | Thr | Leu | Ser | Trp | X | Glu | Ala | Met | Asp | Met | X | Thr | Orn(Pfsb)- | L-6 |
| SEQ ID NO: 60 | | | | | | | | | | | | | | NH₂ | |
| m5-s4.2 | Ac | Thr | Leu | Ser | Trp | X | Glu | Ala | Met | Asp | Met | X | Thr | Dap(Psfb)- | L-6 |
| SEQ ID NO: 61 | | | | | | | | | | | | | | NH₂ | |
| m5-s4.3 | Ac | Thr | Leu | Ser | Trp | X | Glu | Ala | Met | Asp | Met | X | Thr | ^{D}Lys(Psfb)- | L-6 |
| SEQ ID NO: 62 | | | | | | | | | | | | | | NH₂ | |

(X = amino acids linked via the linker unit)

Wherein X is the linking point between the amino acids and the linker; “Ac”=acetyl group; Orn(Pfsb)=N5-(4-(fluorosulfonyl)benzoyl)-L-ornithine; ^{D}Lys(Pfsb)=N-(4-(fluorosulfonyl)benzoyl)-D-lysine; Dap(Pfsb)=(S)-2-amino-3-(4-(fluorosulfonyl)benzamido)propanoic acid; Xaa-NH₂=amino acid with amidated C-terminus; L-6=—CH₂S(CH₂)₃SCH₂—.

In embodiments, the cyclic peptide is a compound of formula (I), wherein R₁=acetyl; the alpha carbon atoms of residue X₅ and X₁₁ are connected together via any of the linker units corresponding to L-1 (=ethyl(2-(4-ethylphenoxy)ethyl)sulfanyl radical), L-4 (bis(methylthio)methanyl radical), L-5 (=1,2-bis(methylthio)ethyl radical), L-6 (=1,3-bis(methylthio)propyl), L-9 (=1,4-bis(methylthio)but-2-enyl radical), or L-10 (=1,2-bis((ethylthio)methyl)phenyl radical) as described above; and residues X₁, X₂, X₃, X₄, X₆, X₇, X₈, X₉, X₁₀, X₁₂, and X₁₃ are as defined in Table 5 below:

TABLE 5

| | AMINO ACID SEQUENCE | | | | | | | | | | | | |
|---|---|---|---|---|---|---|---|---|---|---|---|---|---|
| SEQ ID NO. | X₁ | X₂ | X₃ | X₄ | X₅ | X₆ | X₇ | X₈ | X₉ | X₁₀ | X₁₁ | X₁₂ | X₁₃ |
| SEQ ID NO. 2 | Thr | Leu | Asp | Asp | X | Glu | Glu | Met | Asp | Gly | X | Ser | Asp-NH₂ |
| SEQ ID NO. 3 | Thr | Leu | Asp | Asp | X | Glu | Glu | Met | Asp | Gly | X | Met | Asp-NH₂ |
| SEQ ID NO. 4 | Thr | Leu | Asp | Trp | X | Glu | Glu | Met | Asp | Met | X | Thr | Asp-NH₂ |
| SEQ ID NO. 5 | Thr | Leu | Ser | Trp | X | Glu | Ala | Met | Asp | Met | X | Thr | Asp-NH₂ |

(X = amino acids linked via the linker unit)

In some embodiments, the cyclic peptide provided herein is a compound of formula (I) that comprises an amino acid sequence which is at least about 40%, 50%, 60%, 80%, 90%, or 95% identical to the peptide sequence corresponding to SEQ ID NO: 2, 3, 4, or 5.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds provided herein are within the scope of the invention. Additionally, unless otherwise stated, structures depicted herein are also meant to include compounds that differ only in the presence of at least one isotopically enriched atom. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### 5.3 Synthesis of Cyclic Peptides

The Hedgehog inhibitors described herein are cyclic peptides or peptide derivatives and, as such, these compounds and precursors thereof, can be readily synthesized using well known synthetic methods (e.g., Jones, J., Amino Acid and Peptide Synthesis, Oxford University Press, Oxford, 1992; Bodansky, M., Principles of Peptide Synthesis, Springer-Verlag, Berlin, 1984; Fields et al., Synthetic Peptides: A User's Guide, ed. Grant, W. H. Freeman & Co., New York, N.Y., 1992). Specific synthetic procedures are provided below.

The cyclic peptides described herein can be synthesized using solid-phase methods, such as solid phase peptide synthesis (SPPS). As an example, linear precursors for the cyclic peptides can be synthesized using automated Merrifield techniques of solid phase peptide synthesis using either tBoc or Fmoc chemistry, appropriately protected amino acid building blocks, and an automated peptide synthesizer (e.g., Applied Biosystems (Foster City, Calif.), Model 430A, 431, or 433).

In solid phase peptide synthesis (SPPS) using Fmoc chemistry, the C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with a Fmoc group, which is stable in acid, but removable by base (e.g., piperidine). Side chain functional groups are protected as necessary with base stable, acid labile groups. If necessary, longer precursor peptides can be produced, for example, by conjoining individual synthetic peptides using native chemical ligation or other peptide ligation techniques known in the art. The linear precursor peptide is cyclized while attached to the resin (“on-resin cyclization”) or in solution after cleavage of the peptide chain from the resin (“in-solution cyclization”). The peptide chain is cleaved from the resin under acidic conditions. Depending on the applied condition and the strategy adopted for peptide cyclization, this acid treatment may or may not result also in the removal of the side-chain protecting groups.

The cyclic peptides can be also prepared using solution phase methods. For example, a C-amidated amino acid derivative containing a free amino group is coupled in solution to the carboxyl group of a t-Boc-protected residue using an amide coupling reagent. The t-Boc protecting group is then removed from the resulting dipeptide with acid, a further t-Boc-protected residue is similarly added, followed by similar deblockings and further additions. The N-terminal R group such as acetyl group is added after the last t-Boc removal step and the synthesis is completed, except for deprotecting the Asp residue. The cyclization reaction can be carried out during extension of the peptide chain or after synthesis of the linear peptide precursor.

The assembly of the amino acid building blocks to give the linear peptide precursor can be carried out using standard protocols for amide bond formation. For example, one such protocol involves converting the C-terminal carboxy group of the N-protected amino acid to a mixed anhydride using ethyl chloroformate, phenyl chloroformate, sec-butyl chloroformate, isobutyl chloroformate, or pivaloyl chloride or the like acid chlorides, followed by reaction with a second amino acid (or with a resin-bound amino acid) containing a free amino group. Alternatively, the amide coupling reaction can be carried out by activating the C-terminal carboxy group of the N-protected amino acid in the form of an active ester, such as a 2,4,5-trichlorophenyl ester, a pentachlorophenyl ester, a pentafluorophenyl ester, a p-nitrophenyl ester, a N-hydroxysuccinimide ester, or an ester formed from 1-hydroxybenzotriazole. Another coupling method involves the use of a suitable coupling agent, such as N,N′-dicyclohexylcarbodiimide, N,N′-diisopropylcarbodiimide, HOBt, HAMDU, HOTT, HODT, TOTT, TODT, DMAP, BOP, NOP, PyAOP, AOP, PyBOP, PyNOP, PyBroP, PyCoP, PyFOP, PyTOP, PyPOP, PyDOP, BOP-Cl, ENDPP, HBTU, TBTU, TSTU, TDBTU, HDTU, TPTU, DEPBT, TOTU, PyClU, TPyClU, HAPyU, HBPyU, HPyOPfp, HPySPfp, HAPipU, TAPipU, HOAt, HATU, HDTU, BMC, BEC, BDDC, HOSu, PfpOH, BOMI, DCC, EDC, IBCF, DIC, CIC, CDI, CBMIT, BOI, CIP, CIB, CMBI, BEMT, BEP, BEMT, BMTB, DPPA, DECP, DEPB, DEPC, MPTA, MPTO, DPP-Cl, FDPP, ENDPP, BMP-Cl, NDPP, Cpt-Cl, DEBP, BDP, DEPBO, DOPBO, DOPBT, DEPBT, CDMT, BTC, TFFH, BTFFH, DFIH, BMPI, CMPI, BEP, FEP, BEPH, FEPH, DMTMM, HODhbt, HOCt, PTF, or a combination thereof.

The α-amino group of each amino acid building block employed in the peptide synthesis must be protected during the coupling reaction to prevent side reactions involving their active α-amino function. In addition, reactive side-chain functional groups (e.g. sulfhydryl, amino, carboxyl, and hydroxyl) must be also protected with suitable protecting groups to prevent a chemical reaction from occurring at either the α-amino group site or a reactive side chain site during both the initial and subsequent coupling steps. Suitable protecting groups for peptide synthesis are well known in the art (e.g., see Gross et al., The Peptides: Analysis, Structure, Biology, Vol. 3: “Protection of Functional Groups in Peptide Synthesis”, Academic Press 1981). Alternative protecting groups and conditions for their installation/removal are described in Larock, Comprehensive Organic Transformations, VCH Publishers (1989); Greene and Wuts, Protective Groups in Organic Synthesis, 2d. Ed., John Wiley and Sons (1991); Fieser and Fieser, Fieser and Fieser's Reagentsfor Organic Synthesis, John Wiley and Sons (1994); and Paquette, ed., Encyclopedia of Reagents for Organic Synthesis, John Wiley and Sons (1995), and subsequent editions thereof.

Representative examples of protecting groups that can be used for the preparation of the cyclic peptides provided herein are described in the following. For the α-amino group of any amino acid and the side chain amino group of amino acids such as Lys, Orn or Dab: (a) aromatic urethane-type protecting groups, such as fluorenylmethyloxycarbonyl (Fmoc), carboxybenzyl (Cbz), and substituted Cbz groups (e.g., p-chlorobenzyloxycarbonyl, p-nitrobenzyloxycarbonyl, p-bromobenzyloxycarbonyl, 2,6-dichlorobenzyloxycarbonyl, and the like); (b) aliphatic urethane-type protecting groups, such as Boc, t-amyloxycarbonyl, isopropyloxycarbonyl, 2-(p-biphenyl)-isopropyloxycarbonyl, allyloxycarbonyl and the like; and (c) cycloalkyl urethane-type protecting groups, such as cyclopentyloxycarbonyl, adamantyloxycarbonyl, and cyclohexyloxycarbonyl. For the guanidino group of Arg: Boc, Pbf, 2,2,5,7,8-pentamethylchroman-6-sulfonyl (Pmc), or 2,3,6-trimethyl-4-methoxyphenylsulfonyl group. For the hydroxyl group of Ser, Thr, or Tyr: t-butyl; benzyl (Bzl), or substituted Bzl (e.g., p-methoxybenzyl, p-nitrobenzyl, p-chlorobenzyl, o-chlorobenzyl, and 2,6-dichlorobenzyl) group. For the carboxyl group of Asp or Glu: esterification with benzyl (Bzl), t-butyl, cyclohexyl, and cyclopentyl group, and the like. For the imidazole nitrogen of His: (methyl)trityl, benzyloxymethyl (Bom), and tosyl group. For the phenolic hydroxyl group of Tyr: tetrahydropyranyl, tert-butyl, trityl, Bzl, chlorobenzyl, 4-bromobenzyl, and 2,6-dichlorobenzyl group. For the side chain amido group of Asn or Gln: trityl, xanthyl (Xan). For the thio group of Cys: trityl, p-methoxytrityl, acetamido (Acm), and t-butyl group. For the indolyl group of Trp: Boc,

Regardless of the synthetic method used, the cyclic peptide is typically recovered and purified prior to use using one or a combination separation techniques such as reverse-phase chromatography, solid-phase extraction, normal phase chromatography, precipitation, and the like. These purification methods are well established in the art and will be not dealt with here.

The following section provides examples of specific procedures that can be applied for the preparation of the cyclic peptide disclosed herein. It is understood that the disclosure is not intended to limit the scope of reactions or reaction sequences that are useful in preparing the compounds disclosed herein.

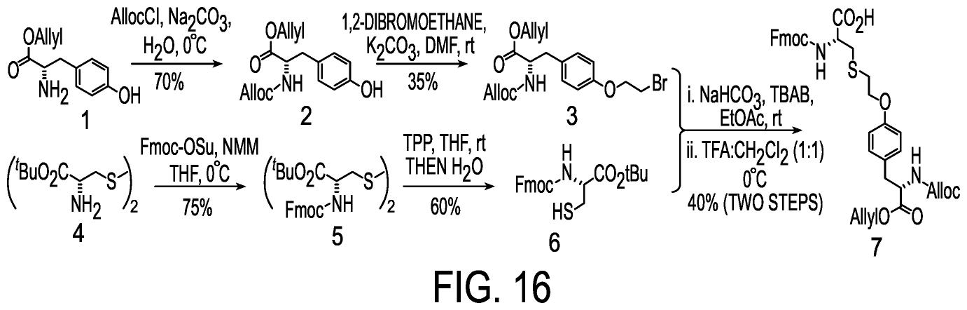

In a first general method exemplified in FIG. 16, a dipeptide building block (7) encompassing a linker unit in the form of

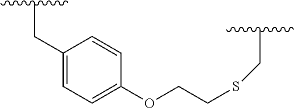 [SMILES: *CSCCOc1ccc(C*)cc1]

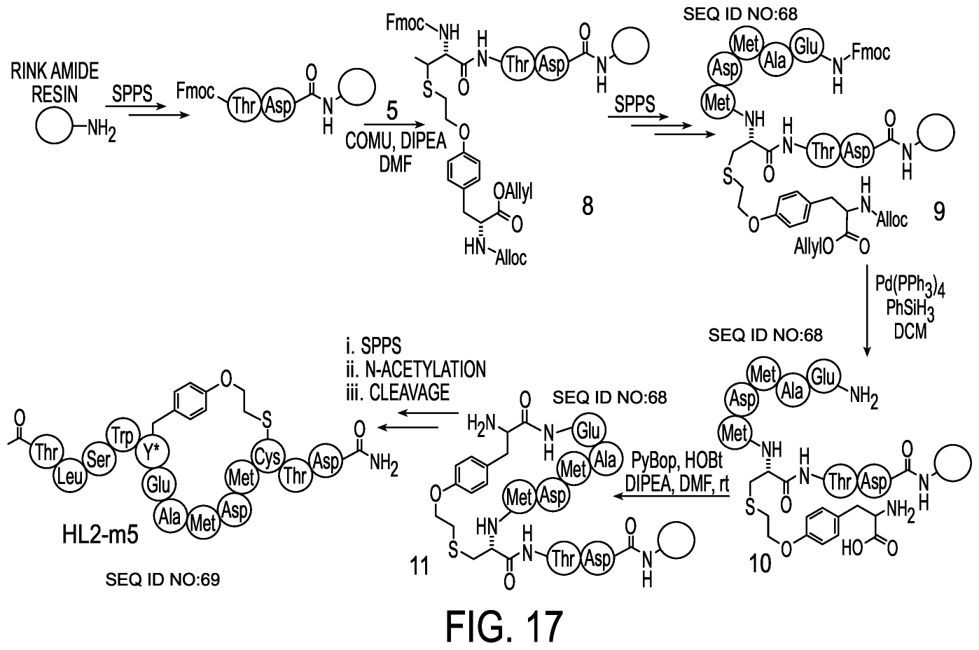

is first prepared via alkylation of N-Alloc-(O-2-bromoethyl)-tyrosine allyl ester (3) with N-Fmoc-(L)-cysteine tert-butyl ester (6), followed by removal of the tert-butyl group under acidic conditions FIG. 16. The dipeptide building block is then incorporated into a linear peptide precursor via solid-phase peptide synthesis, followed by on-resin cyclization and cleavage/deprotection of the peptide from the resin FIG. 17. As shown for the synthesis of cyclic peptide HL2-m5 (Table 1), the first two C-terminal amino acids were loaded on a Rink amide MBHA resin, followed by incorporation of the dipeptide building block (7) via amide coupling with COMU, yielding 8. The peptide chain was then further extended to include amino acid residues Met10 to Glu7, affording 9. The side-chain Alloc and allyl ester protecting groups were then removed using Pd(Ph₃)₄ catalyst in the presence of PhSiH₃, whereas the N-terminal amino group was exposed via Fmoc deprotection with piperidine. On-resin cyclization was then realized under amide coupling conditions with PyBOP and HOBt in the presence of DIPEA as the base, to afford 11. The peptide was then further extended via SPPS to include the N-terminal tail of the peptide, followed by Fmoc deprotection, and acetylation of the N-terminal amino group with acetyl anhydride. The synthesis of HL2-m5 was completed by cleavage of the peptide from the resin using a 95:2.5:2.5 trifluoroacetic acid/triisopropylsilane/water mixture. The desired macrocyclic peptide was obtained via purification by reverse-phase HPLC. This general protocol is useful for the synthesis of cyclic peptides such as, for example, HL2-m1, HL2-m3, and HL2-m5 described in Table 1. A person skilled in the art will appreciate that, by varying the nature of the dipeptide building block, this protocol can be readily adapted and applied to prepare cyclic peptides of formula (I) and (II), wherein the linker unit L₁ and L₂, respectively, is —R₉—S—R₁₀— or —R₉—S—R₁₀— as described above. A person skilled in the art will appreciate that, by varying the nature of the dipeptide building block, this protocol can be readily adapted and applied to prepare cyclic peptides of formula (I) and (II), wherein the linker unit L₁ and L₂, respectively, is —(CH₂)ₙ—Z₁—(CH₂)ₚ— as described above.

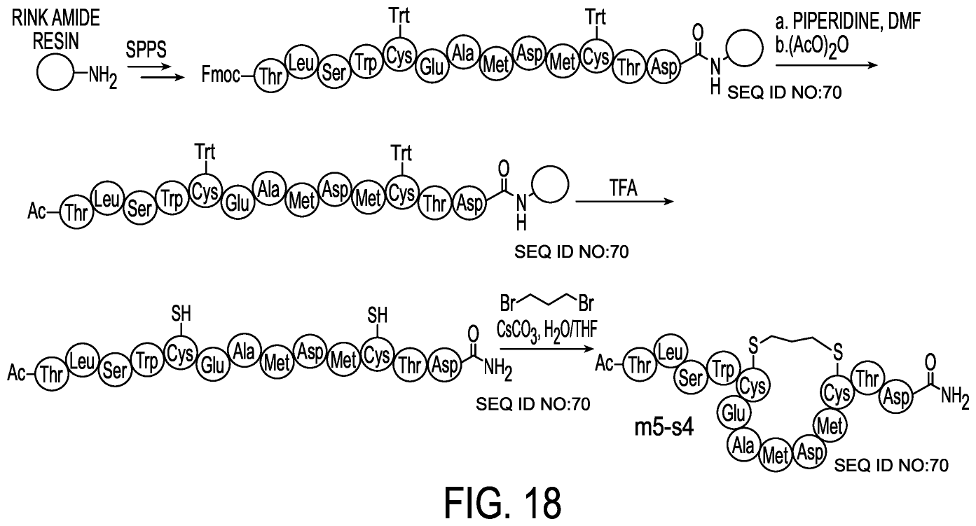

In a second general method exemplified in FIG. 18, the linear peptide precursor is first assembled using Fmoc chemistry via solid-phase peptide synthesis, followed by cyclization in solution. In this method, the two amino acid residues to be linked together are cysteine residues and the cyclization reaction is carried out using a bifunctional cysteine-reactive crosslinking reagent. As illustrated for the synthesis of cyclic peptide m5-s4 (Table 2), the linear peptide precursor was assembled via SPPS followed by N-terminal conjugation with a biotin moiety. After cleavage from the resin with TFA, the linear peptide precursor was precipitated in cold diethyl ether. The cyclization reaction was then performed on the crude peptide via a reaction with 1,3-dibromopropane in water/THF mixture in the presence of TCEP and potassium carbonate. The cyclic peptide product was isolated by semi-preparative reverse-phase HPLC. This general protocol is useful for the synthesis of cyclic peptides such as, for example, m5-s2, m5-s3, m5-s5, as well as their biotinylated counterparts described in Table 2. A person skilled in the art will appreciate that, by varying the nature of cysteine crosslinking reagent, this protocol can be readily adapted and applied to prepare cyclic peptides of formula (I) and (II), wherein the linker unit L₁ and L₂, respectively, is

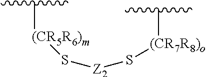

as described above. Similarly, this protocol can be adapted to prepare cyclic peptides in which one or both of the cysteine residues involved in the formation of the intramolecular linkage is/are replaced with another thiol-containing amino acid such as, for example, homocysteine, 5-mercaptonorvaline, penicillamine, D-cysteine, and the like.

In a variation of this protocol, the cysteine-mediated cyclization reaction can be carried out directly on resin. In this case, S-Mmt protected cysteine residues are incorporated into the peptide chain via SPPS. After N-acetylation and with the peptide still bound to the resin, the cysteine side-chain Mmt protecting groups were removed under mildly acid conditions (1% TFA in dichloromethane). On-resin cyclization was the carried out using 1,3-dibromopropane as the cysteine crosslinking reagent in NMP and in the presence of DIPEA as a base. After cyclization, the peptide was cleaved from the resin and purified by HPLC. This protocol is particularly useful whenever it is desirable to introduce an additional cysteine, in addition to those involved in the formation of the intramolecular linkage, into the sequence of the cyclic peptide, e.g., for the purpose of conjugating the cyclic peptide with another molecule or immobilizing on a solid support. In this case, such cysteine residue can be protected with an acid-stable protecting group (e.g., Acm) and appropriately deprotected post-cyclization for further conjugation with a probe, a label, a polymer, a solid support, or another molecule of interest.

Other art-known methods can be applied to prepare cyclic peptides of formula (I) and (II), wherein the linker unit L₁ and L₂, respectively, is —(CH₂)ₙ—Z₁—(CH₂)— as described above. For example, cyclic peptide of this type wherein Z₁ is a disulfide bond (—S—S—) can be prepared via oxidation of two cysteine residues introduced in positions X₅ and X₁₁ for cyclic peptides of formula (I) and in positions X₈ and X₁₁ for cyclic peptides of formula (II). For example, cyclic peptide m5-s1 was prepared in this manner by first assembling the corresponding linear peptide precursor via SPPS, followed by cleavage of the peptide from the resin and disulfide bridge formation by air oxidation in phosphate buffer (pH˜8). Alternative methods for the synthesis of disulfide-bridged cyclic peptides include oxidation of a fully deprotected thiol-containing peptide using potassium ferricyanide or via iodine or thallium trifluoroacetate oxidation of a peptide in which the thiol groups are protected with a benzyl, trityl, or acetamidomethyl protecting group. The oxidation can be performed on either the resin-linked or free peptide. The sulfhydryl protecting groups can be removed during the course of oxidative cyclization as described in Kamber et al., Helv. Chim. Acta, 63(4):899-915 (1980) and Albericio et al. Int. J. Peptide Protein Res., 37:402-413 (1991).

Cyclic peptides provided herein in which the Z₁ is an amide group (i.e., —NHCO— or —CONH—) can be prepared by a variety of well-established methods such as those described in Taylor J. W., Biopolymers, 66, 49-75 (2002). Typically, the cyclization reaction is carried out on-resin via coupling a side-chain deprotected Asp or Glu residue with a side-chain deprotected Lys, Orn, Dab, or Dap residue. In an embodiment, the linear peptide precursor is assembled using SPPS and Fmoc chemistry and using an acid-labile Mtt-protected Lys (or Mtt-protected Orn, Dab, Dap) in combination with an Asp (or Glu) residue protected with a phenylisopropyl ester (OPip) group. The side chains of other Lys, Orn, Dab, and Dap residues in the sequence, if present, are protected with Boc, whereas the side chains of other Asp and Glu residues in the sequence, if present, are protected as t-butyl esters. After assembling the peptide chain, the Mtt and OPip groups are removed with 1% TFA, followed by cyclization under amide coupling conditions. After cyclization, the cyclic peptide is cleaved from the resin and purified.

Cyclic peptides provided herein in which the Z₁ is an olefinic group (i.e., —CH═CH—) or an ethynyl group —(CH₂)₂— can be prepared via a ring-closing olefin metathesis (RCM) reaction according to established synthetic protocols such as those described in Kim Y-W et al., Nature Protocols, 2011, 6:6, 761-771. In this case, the cyclization reaction is carried out between two amino acids containing a side-chain terminal olefin group. Various amino acid building blocks suitable for this chemistry are commercially available (e.g., Anaspec, AAPPTec). In an embodiment, the RCM reaction is carried out on resin-bound, fully protected peptides in 1,2-dichloroethane (DCE) using Grubbs' first-generation catalyst at room temperature. Solution phase synthetic methods for the preparation of cyclic peptides of this type are also available, such as those described in Aihara, K. et al, Org. Lett. 2015, 17, 696-699. When desired, the inter-side-chain olefinic linkage can be reduced postcyclization to give rise to a saturated alkyl linkage (e.g., for cyclic peptides provided herein, where Z₁═—(CH₂)₂—). In an embodiment, the reduction step is carried out via hydrogenation in the presence of Pd/C as the catalysis. After cyclization (and reduction whenever desired), the cyclic peptide is then cleaved from the resin and purified. Various alkenyl group-containing amino acids such as 2-(2′-propenyl)glycine, 2-(3′-butenyl)glycine, 2-(4′-pentenyl)glycine, 2-(5′-hexenyl)glycine, 2-(6′-heptenyl)glycine, 2-(7′-octenyl)glycine, among others, are commercially available and they can be directly used for the synthesis of the cyclic peptides described herein according to the aforementioned protocols. Additional alkenyl group-containing amino acids can be prepared by methods known in the art.

Cyclic peptides provided herein in which the Z₁ is a triazole group can be prepared via a metal-catalyzed or strain-promoted alkyne/azide cycloaddition reaction according to established synthetic protocols such as those described in Kawamoto, S. A. et al, J Med Chem. 2012; 55(3): 1137-1146. In this case, an azide-containing amino acid and an alkyne-containing amino acid are incorporated into the peptide chain via SPPS. On-resin or in solution cyclized is then carried out using in the presence of a copper or ruthenium salts as the catalyst. Since the reaction is highly chemoselective, the cyclization reaction can be performed on a fully protected peptide as well as a fully deprotected peptide. After cyclization, the cyclic peptide is isolated and purified.

Either naturally-occurring and non-naturally-occurring amino acids (or both), as well amino acid analogs and amino acid isosteres can be used in the synthesis of the cyclic peptides provided herein. Any of the synthetic procedures and strategies described above can be adapted to introduce a non-naturally-occurring amino acid, an amino acid analog, or an isostere of a naturally occurring or non-naturally occurring amino acid into the cyclic peptide. As exemplified by the compounds Biot-m5-s7.2 and Biot-m5-s7.4 described in Table 2, an amino acid analog and a non-naturally-occurring amino acid such as Nle and D-cysteine, respectively, were incorporated into the cyclic peptide via SPPS using appropriate N-Fmoc-protected building blocks. In a similar manner, other amino acid analogs such as N-substituted glycine residues can be introduced into the cyclic peptides, as exemplified by Biot-m5-s7.3 in Table 2. N-substituted glycine residues are also referred to as “peptoid unit” and can be synthesized according to well established protocols such as those described in Zuckermann, R. N. et al., Comprehensive Biomaterials (2011), vol. 2, 53-76. To prepare Biot-m5-s7.3, a Fmoc-protected glycine residue was introduced as X₁₀ residue during SPPS synthesis. After Fmoc deprotection with piperidine, the N-terminal amino group was acylated with bromoacetic acid in the presence of N,N-diisopropylcarbodiimide (DIC) as coupling agent. The N-acylated peptide was then reacted with 1-butylamine resulting in the formation of the desired peptoid unit via nucleophilic displacement of the bromoacetyl group by the amine. The rest of the synthesis was completed according to the general procedure described above.

The synthetic procedures and strategies described above can be readily adapted for conjugating the cyclic peptide with another molecule such as, for example, an affinity label molecule, a fluorescent molecule, a (radio)isotopic label molecule, a polymer, a PET or MRI contrast agent, a targeting agent, or a therapeutic agent. For example, after completing the synthesis of desired peptide sequence via SPPS and removal of the N-terminal Fmoc group, a molecule of interest can be conjugated to the N-terminal amino group of the peptide via acylation, alkylation, or reductive amination chemistry. This procedure is exemplified by the synthesis of the biotinylated peptides described in Table 2. Alternatively, amino acid residues carrying protecting groups with orthogonal reactivity can be used. For example, an orthogonally protected amino acid can be introduced into the peptide sequence (e.g., NE-Alloc-Lys, Acm-Cys, Asp(OAllyl)); the orthogonal protecting group is removed selectively under conditions in which all the other protecting groups in the peptide are stable; a molecule is conjugated to the unmasked group (e.g., amino, thiol, carboxylic group) in the partially protected peptide under appropriate reaction conditions. This procedure is exemplified by the synthesis of the fluorosulfonylated peptides described further below. The synthesis of C-terminal conjugated peptide can be achieved using a safety catch resin (e.g., 4-sulfamoylbenzoyl resin). After synthesis of the cyclic peptide on the resin, the resin is activated (typically via alkylation with iodoacetonitrile) followed by reaction with a thiol to release the peptide as a C-terminal thioester. A molecule of interest can be then conjugated to the C-terminus of the peptide via nucleophilic substitution at the thioester group by means of an amine, hydrazine, or aminothiol group.

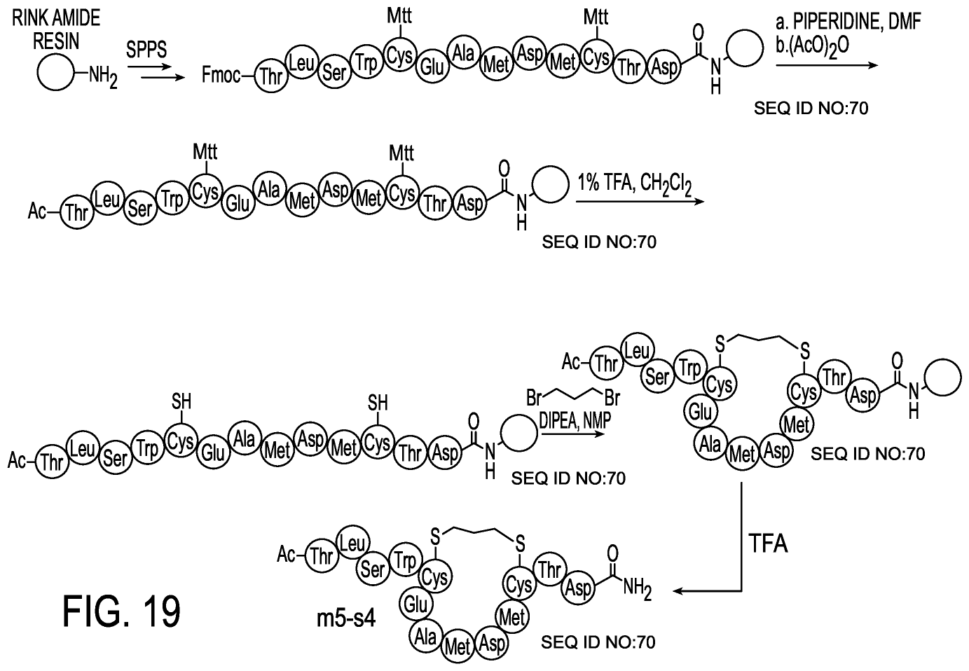

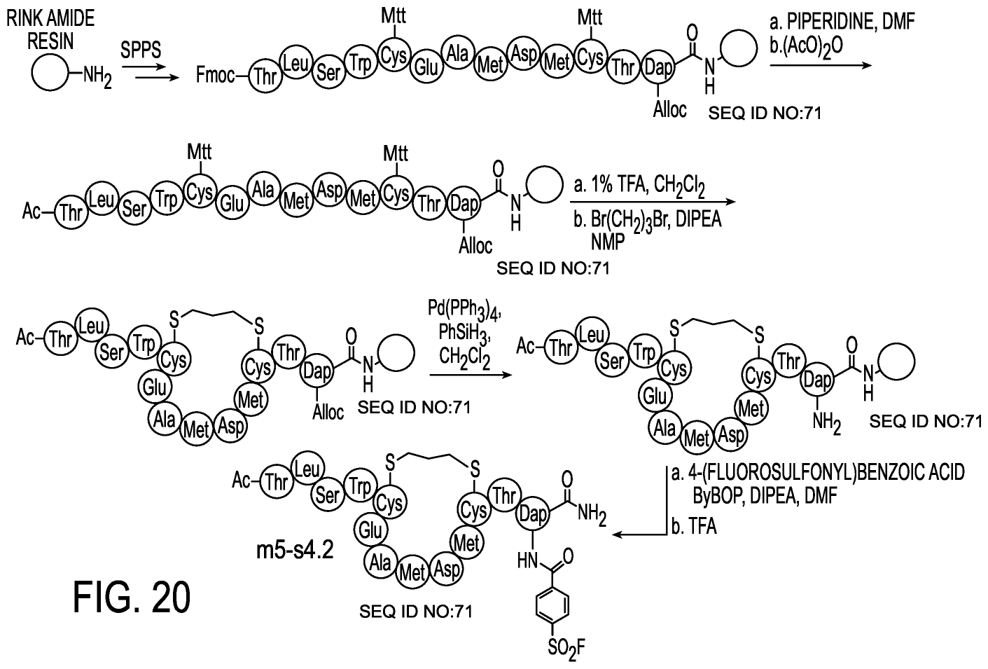

The synthetic procedures and strategies described above can be readily adapted also to introduce a warhead into the cyclic peptide. An exemplary procedure is provided in FIG. 20, which refers to the synthesis of peptide m5-s4.1 described in Table 4. In this case, a cyclic peptide was first prepared via SPPS and Fmoc chemistry, followed by on-resin cyclization via cysteine alkylation with 1,3-dibromopropane according to the general procedure described earlier (FIG. 19). During peptide assembly, an Alloc-protected ornithine residue was introduced as the C-terminal amino acid. Post-cyclization, the Alloc protecting group was selectively removed with Pd(Ph₃)₄ catalyst in the presence of PhSiH₃ to unmask the side-chain amino group of the ornithine residue. The side-chain amino group was then acylated with 4-(fluorosulfonyl)benzoic acid in the presence of PyBop and DIPEA. The cyclic peptide product was then cleaved from the resin and purified to yield the desired cyclic peptide containing a warhead in the form of fluorosulfonyl group. Using a similar protocol, other fluorosulfonyl group-containing peptides such as m5-s4.2 and m5-s4.3 were prepared. A person skilled in the art will appreciate how this protocol can be readily adapted to introduce this and other types of warhead groups into various positions within the cyclic peptide molecule.

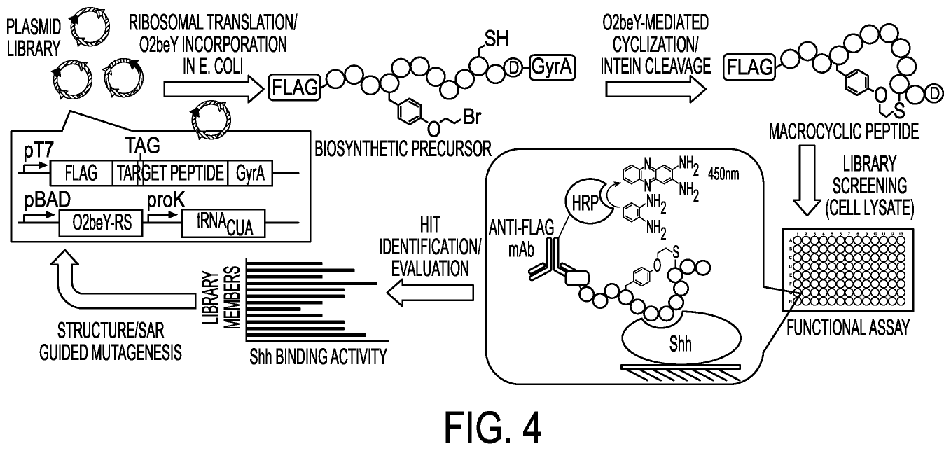

While chemical synthesis is one method for preparing the Hedgehog inhibitors described herein, selected cyclic peptides provided herein can be prepared also by recombinant methods. As shown in FIG. 4, one such method involves the incorporation of the non-canonical amino acid O-2-bromoethyl-tyrosine (O2beY) into a recombinant peptide via amber stop codon suppression by means of an engineered aminoacyl-tRNA synthetase. Upon expression and ribosomal synthesis of the precursor polypeptide in the cell (E. coli), O2beY reacts with a proximal cysteine residue in the peptide sequence, resulting in the formation of a side-chain-to-side-chain cyclic peptide. Depending on the relative position of the O2beY/Cys pair within the peptide sequence, the cyclic peptide features an inter-side-chain linkage in the form of

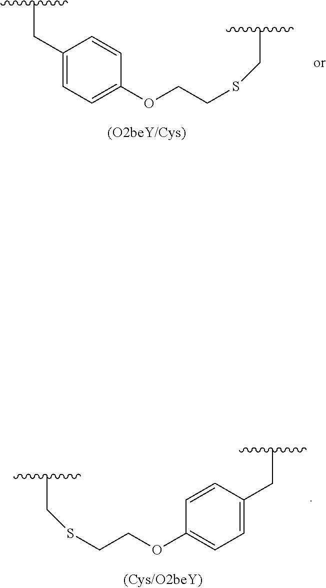 [SMILES: *CSCCOc1ccc(C*)cc1 ; *CSCCOc1ccc(C*)cc1]

Peptide or protein based tags such as, for example a FLAG tag or an intein tag can be fused to the N-terminus or C-terminus of the cyclic peptide by engineering the gene that encodes for the peptide. Similarly, various permutations of the amino acid sequence encompassed by the cyclic peptide can be carried out by site-directed or site-saturation mutagenesis of the peptide-encoding gene. When the peptide is produced in an expression host, these sequence permutations will be limited to mostly naturally occurring amino acids. When the peptide is produced via a cell-free expression system, sequence permutations will be normally limited to amino acids and amino acid analogs that are accepted by the ribosome. Cyclic peptides containing different types of thioether-based linkages can be obtained by varying the nature of the cysteine-reactive non-canonical amino acid. Prerequisites in this regard are that the non-canonical amino acid must be sufficiently stable in a biological milieu (e.g., cell culture or lysate), a suitable aminoacyl-tRNA synthetase for the genetic incorporation of such amino acid is available or can be engineered, and that non-canonical amino acid is accepted by the ribosome during ribosomal synthesis. A person skilled in the art will recognize that the recombinant methods described above can be applied to prepare only certain types of the cyclic peptides provided herein. A specific advantage of this approach is the possibility to produce large collections of the cyclic peptides featuring various sequence permutations with naturally occurring amino acids. As illustrated in FIGURE 4, this capability can be exploited to optimize the functional properties of the cyclic peptide (e.g., Shh binding affinity), e.g. via the screening of libraries of variants of such peptide. Further modulation of the biological and physicochemical properties of the cyclic peptide can by made by producing the same or an analogous peptide by chemical synthesis and by means of chemical modifications introduced according to the procedures described above.

### 5.4 Biological Activity of Cyclic Peptides

As for any peptide/protein interaction, various analytical and biophysical methods can be applied to test and assess the ability of the compounds provided herein to bind to Hedgehog proteins. These methods include, but are not limited to immunoassays (e.g., ELISA), radio(immune)assays, fluorescence-based assays, colorimetric assays, and/or techniques such as surface plasmon resonance (SPR), FRET, fluorescence polarization, isothermal calorimetry, NMR, and the like. A method that can be used is a solid phase immunoassay, in which the Hedgehog protein is immobilized on a plate or test tube and then incubated with the peptide at a fixed or variable concentration. In this assay, the peptide is functionalized with a label in the form of an antibody epitope (e.g., ‘FLAG tag’ peptide sequence) or an affinity tag molecule (e.g., biotin). After washing the unbound peptide, the Hedgehog-bound peptide is detected and quantified by means of a colorimetric assay using an enzyme-conjugated antibody (e.g., horseradish peroxidase-conjugated anti-FLAG antibody) or enzyme-conjugated capture protein (e.g., horseradish peroxidase-conjugated streptavidin). This assay measures the direct binding of the peptide to the protein and it can be applied to measure both the relative binding affinity of the cyclic peptide compared to a reference molecule (e.g., a linear HHIP L2-derived peptide) as well as the equilibrium dissociation constant (K_{D}) for the interaction between the cyclic peptide and the Hedgehog protein.

The immunoassay described above can be adapted to serve as a competition assay. In this case, a fixed amount of the labeled peptide is incubated with the immobilized Hedgehog protein in the presence of a test compound (e.g., another cyclic peptide or a small-molecule compound). The test molecule competes with the cyclic peptide for binding to the immobilized Hedgehog protein. The amount of bound peptide is quantified using the colorimetric assay described above and the extent of inhibition in the presence of the test compound is compared to that in the absence of it. From the resulting dose-response curves, a IC₅₀ value for the test compound can be obtained. Using this assay and the cyclic compounds provided herein, the Hedgehog inhibitory activity of a test molecule can be evaluated. Since the cyclic peptides described herein inhibit the signaling activity of Hedgehog proteins, these compounds can be used to screen and identify compounds that are able to inhibit the signaling function of Hedgehog proteins.

The ability of the compounds provided herein to inhibit ligand-induced activation of the Hedgehog pathway in a cell can be assessed by means of a cell based assay. In an exemplary procedure, a suitable reporter plasmid is introduced in a Hedgehog responsive mammalian cell. Typically, the reporter plasmid contains a reporter gene encoding (e.g., a gene encoding for firefly luciferase) under a Gli-controlled promoter, thereby providing a reporter gene that is responsive to the activity of the Gli transcription factors. Typically, a second control plasmid is introduced in the cell that contains a second reporter gene (e.g., Renilla luciferase gene) under a constitutive promoter for signal normalization purposes. The reporter cells are incubated with the Hedgehog protein in the presence and in the absence of the cyclic peptide. The extent of Hedgehog pathway suppression can be determined based on relative activity of the luciferase reporter enzyme, which in turn can be determined by measuring the luminescence signal after addition of a luminogenic substrate of the luciferase enzyme.

The ability of the compounds to suppress a transcriptional response associated with activation of the Hedgehog pathway can be assessed by incubating a Hedgehog responsive mammalian cell with the Hedgehog ligand in the presence and in the absence of the cyclic peptide inhibitor. The messenger RNA (mRNA) is extracted and a DNA the mRNA (cDNA) is produced via reverse transcription. The transcriptional level of canonical Hedgehog target genes such as Gli1 and Ptch1 is then determined via real-time PCR and/or via PCR followed by DNA gel electrophoresis. From the resulting dose-response curves, the activity of the cyclic peptide toward suppressing Hedgehog induced transcriptional activity can be established and quantitatively measured by means of IC₅₀ values.

The ability of the cyclic peptides to inhibit proliferation and reduce viability of cancer cell can be measured using protocols well established in the art. For example, the antiproliferative activity of these compounds can be measured using the commercially available Click-iT EdU cell proliferation assay. In these case, cells treated with the Hedgehog ligand and cyclic peptide inhibitor are incubated with 5-ethynyl-2′-deoxyuridine (EdU), which is incorporated into the cell during DNA synthesis. The amount of viable cells in a sample is then determined by means of fluorescently labeled secondary reagent followed by flow cytometry analysis. The cytotoxicity of the compounds can be measured using well established cell viability assays such as the MTT (3-(4,5-dimethylthiazol-2-yl)-2,5-diphenyltetrazolium bromide) assay. In live cells, MTT is converted into a colored metabolite (formazan), providing a readout of the amount of viable cells in a sample. Among others, the aforementioned assays can be used to evaluate the potency of the cyclic peptides toward inhibiting proliferation/viability of a cell, e.g., a cancer cell. Methods for assessing and evaluating the in vivo potency of a compound (e.g., anticancer activity of the compound in a xenograft model of the tumor) are well known in the art and will not be dealt with here.

### 5.5 Applications and Methods of Use

Applications of the cyclic peptide Hedgehog inhibitors provided herein include in vitro and in vivo applications. The cyclic peptide Hedgehog inhibitors provided herein may be particularly useful in the treatment of diseases or conditions, whose development or progression is associated with physiological or aberrant stimulation of the Hedgehog pathway.

The compounds provided herein may be particularly useful in the treatment of neoplasms or other proliferative diseases in a patient affected by such disease(s). As used herein, the terms “cancer”, “proliferative disease” and “neoplasms” refer to a condition in which cells have the capacity for autonomous growth, i.e., an abnormal state or condition characterized by rapidly proliferating cell growth. Proliferative and neoplastic disease states may be categorized as pathologic, i.e., characterizing or constituting a disease state, or may be categorized as non-pathologic, i.e., a deviation from normal but not associated with a disease state.

The compounds provided herein may also be useful in the treatment of pulmonary diseases and developmental disorders in a patient affected by such disease(s). In addition, the compounds described above may also be used in vitro for research, diagnostic, or clinical purposes such as, for example, for determining the presence and/or expression levels of Hedgehog proteins in a sample or a tissue, for determining the susceptibility of a patient's disease to inhibition of the Hedgehog pathway by means of a compound of formula (I) or (II), for elucidating the role of Hedgehog pathway signaling in a cellular pathway or process, and the like.

In some embodiments, compounds provided herein are used to inhibit the signaling function of at least one homolog of Hedgehog, i.e., Shh, Ihh, and/or Dhh. In some embodiments, the compounds provided herein are used to inhibit the Hedgehog pathway in cells. In some embodiments, the cells are wild type. In some embodiments, the cells are subject to at least one genetic modification. In some embodiments, the cells are cancer cells.

In some embodiments, compounds are provided for use as therapeutics. In some embodiments, a method is provided of treating a proliferative disease in a subject suffering therefrom, the method comprising administering to the subject a therapeutically effective amount of the cyclic peptide Hedgehog inhibitor. In certain embodiments, the proliferative disease is a benign neoplasm. In certain embodiments, the proliferative disease is cancer.

The cyclic peptide described herein may be used in the treatment or prevention of neoplasms. In certain embodiments, the neoplasm is a benign neoplasm. In other embodiments, the neoplasm is a malignant neoplasm.

In some embodiments, the cancer is a hematopoietic neoplastic disorders. As used herein, the term “hematopoietic neoplastic disorders” includes, but is not limited to, diseases involving hyperplastic/neoplastic cells of hematopoietic origin, e.g., arising from myeloid, lymphoid or erythroid lineages, or precursor cells thereof. Diseases arising from poorly differentiated acute leukemias include, but are not limited to erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML); lymphoid malignancies include, but are not limited to acute lymphoblastic leukemia (ALL) which includes, but is not limited to, B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Steinberg disease.

In certain embodiments, the cancer is a solid tumor. Exemplary solid cancers that may be treated using the cyclic peptides described herein include colon cancer, lung cancer, bone cancer, pancreatic cancer, stomach cancer, esophageal cancer, skin cancer, brain cancer, liver cancer, ovarian cancer, cervical cancer, uterine cancer, testicular cancer, prostate cancer, bladder cancer, kidney cancer, neuroendocrine cancer, breast cancer, gastric cancer, eye cancer, gallbladder cancer, laryngeal cancer, oral cancer, penile cancer, glandular tumors, rectal cancer, small intestine cancer, sarcoma, carcinoma, melanoma, urethral cancer, vaginal cancer, to name but a few. In some embodiments, the cyclic peptides provided herein are used to treat leukemia. In some embodiments, the cyclic peptides provided herein are used to treat lymphoma. In some embodiments, the cyclic peptides provided herein are used to treat pancreatic cancer. In some embodiments, the cyclic peptides provided herein are used to treat lung cancer. In some embodiments, the cyclic peptides provided herein are used to treat breast cancer. In some embodiments, the cyclic peptides provided herein are used to treat colon cancer.

In some embodiments, the compounds of formula (I) or (II) block or inhibit paracrine Hedgehog signaling, for example by inhibiting the activation of the Hedgehog pathway in the surrounding stroma by Hedgehog ligand-secreting tumor cells. This paracrine mode of Hedgehog pathway activation in the stroma surrounding the tumors has recently been shown to create a favorable environment for tumor growth (Yauch, Gould et al. 2008; Theunissen and de Sauvage 2009; Tian, Callahan et al. 2009)

In some embodiments, the compounds of formula (I) or (II) block or inhibit autocrine Hedgehog signaling, for example by inhibiting the activation of Hedgehog pathway in a tumor cells by Hedgehog ligand(s) secreted by the same or other tumor cells. An autocrine mode of Hedgehog pathway activation has also been associated with tumor growth and proliferation (Rubin and de Sauvage 2006).

In some embodiments, the above-mentioned cancer is associated with abnormal Hedgehog signaling, e.g., abnormal/aberrant activation of the Hedgehog pathway mediated by the Hedgehog ligands. Examples of cancers known to be linked to abnormal or aberrant Hedgehog signaling include basal cell carcinoma, medulloblastoma, rhabdomyosarcoma, glioma, breast cancer, esophageal cancer, gastric cancer, pancreatic cancer, prostate cancer, small-cell lung cancer, biliary tract cancer, bladder cancer and oral cancer (Varjosalo and Taipale 2008). Additionally, tumor types such as small-cell lung, gastric, pancreatic, and prostate cancer have been reported to display abnormal activation of the Hedgehog pathway in the absence of known mutations (Peukert and Miller-Moslin 2010).

In some embodiments, the above-mentioned cancer/tumor is associated with overexpression or increased/abnormal activity of the Patched receptor.

In another embodiment, the above-mentioned cancer is a brain tumor (e.g., brain ependynoma, a medulloblastoma), an ovary tumor (e.g., ovary carcinoma, endometriosis or adenocarcinoma), a breast tumor (e.g., breast fibroadenoma or carcinoma), a glioblastoma, a skin tumor (e.g., skin melanoma), a meningioma, an astrocytoma, a liver tumor (e.g., liver cholangiocarcinoma), a prostate carcinoma, basal cell carcinoma, gastrointestinal cancers (e.g., gastric cancer), esophageal cancer, a bladder tumor (e.g., bladder adenocarcinoma), a lung tumor, hematological malignancies (e.g., lymph node lymphoma), a vascular endothelium hemangioma, pancreatic cancer a kidney carcinoma or a thyroid follicular adenoma.

In another aspect, a method is provided of preventing or delaying cancer development in a subject (e.g., in a subject at risk of developing cancer), said method comprising administering to said subject an effective amount of the cyclic peptide Hedgehog inhibitors described here.

The cyclic peptide Hedgehog inhibitors described here may be used in the treatment or prevention of an inflammatory disease. In certain embodiments, the inflammatory disease is an autoimmune disease. Examples of autoimmune diseases include, but are not limited to, acute disseminated encephalomyelitis (ADEM), Addison's disease, ankylosing spondylitis, antiphospholipid antibody syndrome (APS), autoimmune hemolytic anemia, autoimmune hepatitis, autoimmune inner ear disease, Bechet's disease, bullous pemphigoid, coeliac disease, Chagas disease, Churg-Strauss syndrome, chronic obstructive pulmonary disease (COPD), Crohn's disease, dermatomyositis, diabetes mellitus type 1, endometriosis, Goodpasture's syndrome, Graves' disease, Guillain-Barre syndrome (GBS), Hashimoto's disease, Hidradenitis suppurativa, idiopathic thrombocytopenic purpura, inflammatory bowel disease (IBD), interstitial cystitis, lupus erythematosus, morphea, multiple sclerosis, myasthenia gravis, narcolepsy, neuromyotonia, pemphigus vulgaris, pernicious anaemia, Polymyositis, polymyalgia rheumatica, primary biliary cirrhosis, psoriasis, rheumatoid arthritis, schizophrenia, scleroderma, Sjogren's syndrome, temporal arteritis (also known as “giant cell arteritis”), Takayasu's arteritis, Vasculitis, Vitiligo, and Wegener's granulomatosis. Examples of other types of inflammatory diseases include, but are not limited to, allergy including allergic rhinitis/sinusitis, skin allergies (urticaria/hives, angioedema, atopic dermatitis), food allergies, drug allergies, insect allergies, and rare allergic disorders such as mastocytosis, asthma, arthritis including osteoarthritis, rheumatoid arthritis, and spondyloarthropathies, primary angitis of the CNS, sarcoidosis, organ transplant rejection, fibromyalgia, fibrosis, pancreatitis, and pelvic inflammatory disease.

The cyclic peptide Hedgehog inhibitors described here may be used in the treatment or prevention of conditions of the eye. In certain embodiments, this condition is diabetic retinopathy.

In some embodiments, the cyclic peptide Hedgehog inhibitors described here may be used in the treatment of interstitial pneumonitis. In some embodiments, the cyclic peptide is used in the treatment of interstitial pulmonary fibrosis. In some embodiments, the pulmonary fibrosis is bleomycin-induced. In some embodiments, the pulmonary fibrosis is not bleomycin-induced.

In certain embodiments, the cyclic peptide Hedgehog inhibitors described here may be used in the treatment of developmental disorders. In certain embodiments, a method is provided of treating a developmental disorder, the method comprising administering to the subject a therapeutically effective amount of a compound of formula (I) or (II). In some embodiments, the developmental disorder is phocomelia. In some embodiments, the developmental disorder is cyclopia. In some embodiments, the developmental disorder is treated at a pre-natal stage. In some embodiments, the developmental disorder is treated in an intrauterine fashion. In some embodiments, the developmental disorder is treated at a post-natal stage. In some embodiments, the subject is human. In some embodiments, the subject is a human embryo.

Suppression of Hedgehog signaling has been found to be beneficial for exerting protective effect on neurons, e.g., as described in Kottmann, A. H., PCT App. WO2010117800. Thus, inhibition of the Hedgehog pathway can be useful in the treatment of a subject afflicted with or at risk of developing a neurodegenerative disorder. Among the possible mechanisms, the neuroprotective effect of Hedgehog pathway inhibition has been linked to an increase in glial cell-derived neurotrophic factor (GDNF), resulting in the protection of the neurons. In some embodiments, compounds of formula (I) or (II) may be used in the treatment or prevention of neurodegenerative disorders. In some embodiments, the neurodegenerative disorder is Parkinson's Disease (PD), Amyotrophic Lateral Sclerosis (ALS), Alzheimer's Disease (AD), Supra Nuclear Palsy, spinocerebellar ataxias, multiple system atrophy, or corticobasal degeneration.

Suppression of Hedgehog signaling has been found to be beneficial also for improving blood vessel density and blood vessel patency in a tissue, e.g., as described in Olive K. P. et al., US Pat. App. 2012/0020876). In turn, Hedgehog signaling inhibition can be useful, for example, for increasing the blood flow to an ischemic tissue and for increasing drug delivery and/or radiation penetration to a disease-affected tissue, e.g., a tumor tissue. In some embodiments, the cyclic peptide Hedgehog inhibitors described here may be used for improving blood vessel density and/or blood vessel patency in a tissue. In some embodiments, the tissue is an ischemic tissue, a tumor tissue, a non-tumor tissue, or a poorly permeable tissue. In some embodiments, compounds of formula (I) or (II) described here may be used for improving the delivery of an exogenous or an endogenous agent to a tissue. In some embodiments, compounds of formula (I) or (II) may be used for improving the delivery of therapeutic agent to a tissue. In some embodiments, compounds of formula (I) or (II) may be used for improving the delivery of an anticancer agent to a tissue. Examples of anticancer agents that can be used in combination with a compound of formula (I) or (II) in the context of this application are provided further below. In some embodiments, compounds of formula (I) or (II) may be used for improving the delivery of an imaging agent to a tissue. In certain embodiments, the imaging agent is a magnetic resonance imaging (MRI) contrast agent, computerized axial tomography (CAT) contrast agent, or positron emission tomography (PET) contrast agent. In certain embodiments, the compound provided herein and the agent to be delivered to the tissue are administered concurrently. In certain embodiments, the compound provided herein and the agent to be delivered to the tissue are administered sequentially.

In some embodiments, a method is provided of controlling stem cell differentiation, wherein the method comprises contacting at least one stem cell with a cyclic peptide inhibitor of Hedgehog protein(s) provided herein. In some embodiments, the resulting differentiation is terminal. In some embodiments, the resulting differentiation is non-terminal. In some embodiments, differentiation is from a stem cell to an intermediate cell stage (such as a progenitor cell). In some embodiments, differentiation is from a progenitor cell to a more specialized cell. In some embodiments, differentiation is from a definitive endoderm cell to a pancreatic precursor cell. In some embodiments, differentiation is from a stem cell to an endoderm cell. In some embodiments, a method is provided of controlling cell dedifferentiation, wherein the method comprises contacting at least one cell with a cyclic peptide provided herein, thereby resulting in a stem cell.

In certain embodiments, the cyclic peptides described herein are used to control cell differentiation. In certain embodiments, the differentiation is related to cellular development. In certain embodiments, the differentiation is related to organ development. In certain embodiments, the differentiation is related to organ system development. In certain embodiments, the differentiation is related to organism development.

In some embodiments, cyclic peptide inhibitor of Hedgehog protein(s) provided herein may be used to block hair regrowth. In certain embodiments, a compound of formula (I) or (II), or a composition thereof, may be applied to the skin post-depilation to prevent or slow hair regrowth. In some embodiments, a compound of formula (I) or (II), or a composition thereof, may be applied to the skin post-depilation to prevent or slow hair follicle development.

In certain embodiments, compounds and pharmaceutical compositions provided herein can be employed in combination therapies, that is, the compounds provided herein, or pharmaceutical compositions thereof, can be administered concurrently with, prior to, or subsequent to, at least one other desired therapeutic or medical procedure. The combination of prophylactic/therapeutic agents and/or compositions provided herein may be administered or co-administered (e.g., consecutively, simultaneously, at different times) in any conventional dosage form. Co-administration, in the context of the present disclosure, refers to the administration of more than one prophylactic or therapeutic agent in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the at least one active agent(s) is used/administered in combination with at least one agent(s) currently used to prevent or treat the disorder in question (e.g., an anticancer agent). The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a compound provided herein may be administered concurrently with another anticancer agent), or they may achieve different effects (e.g., control of any adverse effects).

For example, other therapies or anticancer agents that may be used in combination with the compounds provided herein include surgery, radiotherapy (γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, cyclophosphamide, melphalan, ifosfamide), antimetabolites (methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. Other chemotherapeutic agents include as 13-cis retinoic acid (RA), histone deacetylase inhibitor (SAHA), and kinase inhibitors. Additionally, the present invention also encompasses the use of certain cytotoxic or anticancer agents currently in clinical trials and which may ultimately be approved by the FDA (including, but not limited to, epothilones and analogues thereof and geldanamycins and analogues thereof). For a more comprehensive discussion of updated cancer therapies and a list of the FDA approved oncology drugs, one can refer to www.nci.nih.gov, www.fda.gov/cder/cancer/druglistframe.htm, and The Merck Manual, Seventeenth Ed. 1999, the entire contents of which are hereby incorporated by reference.

In some embodiments, at least one compound provided herein is used in combination with at least one of the aforementioned anticancer agents. In other embodiments, at least one compound provided herein is used in combination with another inhibitor of the Hedgehog pathway. Inhibitors of the Hedgehog pathway that can be used in combination with the compounds provided herein include, among others, Smo antagonists such as cyclopamine, HhAntag691, GDC-0449, IPI-926 and LDE225, the Shh inhibitor Robotnikinin, and the Gli inhibitors GANT61 and JK184. Other Hedgehog pathway inhibitors are described in (Peukert and Miller-Moslin 2010; Stanton and Peng 2010; Sharpe, Wang et al. 2015). The combination of the Hedgehog inhibitors described herein with inhibitors targeted against other components of the Hedgehog pathway can provide an additive or synergistic effect toward suppressing Hedgehog pathway signaling in a normal cell or cancer cell.

In certain embodiments, inventive compounds are useful in treating a subject in clinical remission. In some embodiments, the subject has been treated by surgery and may have limited unresected disease.

The amount of the agent or pharmaceutical composition which is effective in the prevention and/or treatment of a particular disease, disorder or condition (e.g., cancer) will depend on the nature and severity of the disease, the chosen prophylactic/therapeutic regimen (i.e., compound, cells), the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.001 to 1000 mg/kg of body weight/day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a patient, in the context of the present disclosure, should be sufficient to effect a beneficial prophylactic and/or therapeutic response in the patient over time. The size of the dose will also be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

The terms “treat/treating/treatment” and “prevent/preventing/prevention” as used herein, refers to eliciting the desired biological response, i.e., a therapeutic and prophylactic effect, respectively. The therapeutic effect may comprise at least one of a decrease/reduction in tumor, a decrease/reduction in the severity of the cancer (e.g., a reduction or inhibition of metastasis development), a decrease/reduction in symptoms and cancer-related effects, an amelioration of symptoms and cancer-related effects, and an increased survival time of the affected host animal, following administration of the agent/composition provided herein. A prophylactic effect may comprise a complete or partial avoidance/inhibition or a delay of cancer development/progression (e.g., a complete or partial avoidance/inhibition or a delay of metastasis development), and an increased survival time of the affected host animal, following administration of a compound provided herein. Similar considerations can be made in the context of the treatment or prevention of other diseases or conditions, for which the compounds provided herein can provide a pharmacological benefit. As described above, these include, for example, an inflammatory disease, a developmental disorder, and a proliferative disease.

In another aspect, “pharmaceutically acceptable” compositions are provided, which comprise a therapeutically effective amount of at least one of the compounds described herein, formulated together with at least one pharmaceutically acceptable carrier (additive) and/or diluent. As described in detail, the pharmaceutical compositions provided herein may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

In some embodiments, the compound provided herein is in the form of a salt, e.g., a pharmaceutically acceptable salt. As used herein the term “pharmaceutically acceptable salt” refers to salts of compounds that retain the biological activity of the parent compound, and which are not biologically or otherwise undesirable. Pharmaceutically acceptable salts are well known in the art and they are described, for example, in Berge et al., J. Pharmaceutical Sciences, 1977, 66, 1-19, which is incorporated herein by reference. Such salts can be prepared in situ during the final isolation and purification of the cyclic peptide, or may be prepared separately by reacting a free base function with a suitable acid. Pharmaceutically acceptable acid addition salts may be prepared from inorganic and organic acids. Representative acid addition salts include, but are not limited to acetate, adipate, alginate, citrate, aspartate, benzoate, benzenesulfonate, bisulfate, butyrate, camphorate, camphor sulfonate, decanoate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, octanoate, oxalate, palmitoate, pectinate, persulfate, 3-phenylpropionate, picrate, pivalate, propionate, succinate, tartrate, thiocyanate, phosphate, glutamate, bicarbonate, ptoluenesulfonate, and undecanoate. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid. Basic addition salts also can be prepared by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like. Salts derived from organic bases include, but are not limited to, salts of primary, secondary and tertiary amines.

In certain embodiments, the present disclosure provides a method for the inhibition of Hedgehog ligand-induced transcription in cells, the method comprising contacting said cells with an effective amount of a compound of formula (I) or (II). In certain embodiments, a compound of formula (I) or (II) inhibits the Hedgehog pathway upstream of Patched. In certain embodiments, a compound of formula (I) or (II) inhibits the Hedgehog pathway by preventing binding of at least one homolog of the Hedgehog protein to the Patched receptor.

In another aspect, a method is provided for the identification and characterization of an agent that is capable of inhibiting at least one analog of Hedgehog, i.e., Shh, Ihh, and/or Dhh. For example, such method may comprise adding a test agent to a reaction mixture containing a cyclic peptide of formula (I) or (II) and at least one analog of Hedgehog, i.e., Shh, Ihh, and/or Dhh. Binding between the cyclic peptide and the Hedgehog protein(s) is then determined and compared to the binding of the cyclic peptide to the Hedgehog protein(s) in the absence of the test agent. A lower binding in the presence of the test agent is indicative that such agent may be useful for inhibiting Hedgehog protein-mediated signaling in a cell.

Determination of the binding interaction between the cyclic peptide and the Hedgehog protein(s) can be monitored by any number of means including, but not limited to an immunoassay, a radio(immune)assay, a fluorescence-based assay, a colorimetric assay, and/or techniques such as surface plasmon resonance (SPR), FRET, fluorescence polarization, and an alpha-screen. The above-mentioned methods may be performed in a cell-free or a cell-based assay. Representative examples of suitable assays for measuring binding of the cyclic peptides described herein and the Hedgehog protein(s) are provided in Examples 1 and 8. Example 8 further describes a procedure involving the use of the compounds described herein for measuring the ability of another agent to bind Hedgehog proteins.

Kits or packages are also provided comprising a compound provided herein and/or a pharmaceutical composition thereof. In certain embodiments, such kits comprise a compound provided herein useful for inhibiting Hedgehog ligand-mediated Hedgehog pathway signaling in vitro, in a cell, or in an animal. The kit optionally comprises instructions for prescribing and directing the use of a compound provided herein. In certain embodiments, such kits comprises a compound provided herein useful for preventing and/or treating cancer. In certain embodiments, such kits comprise the combination of a compound provided herein and another chemotherapeutic agent. The agents may be packaged separately or together. The kit optionally includes instructions for prescribing the medication. In certain embodiments, the kit includes multiple doses of each agent. The kit may include sufficient quantities of each component to treat a subject for a week, two weeks, three weeks, four weeks, or multiple months. The kit may include a full cycle of chemotherapy. In certain embodiments, the kit includes multiple cycles of chemotherapy.

### 5.6. Formulations

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Compounds provided herein may be combined with a pharmaceutically acceptable carrier to form a pharmaceutical composition. In certain embodiments, the pharmaceutical composition includes a pharmaceutically acceptable amount of an inventive compound. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, from about 5% to about 70%, or from about 10% to about 30%.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations provided herein include those suitable for oral, nasal, topical (including buccal and sublingual), rectal, vaginal and/or parenteral administration. The formulations may conveniently be presented in unit dosage form and may be prepared by any methods well known in the art of pharmacy. In certain embodiments, a formulation provided herein comprises an excipient selected from the group consisting of cyclodextrins, liposomes, micelle forming agents, e.g., bile acids, and polymeric carriers, e.g., polyesters and polyanhydrides; and a compound provided herein. In certain embodiments, an aforementioned formulation renders orally bioavailable a compound provided herein.

Methods of preparing these formulations include the step of bringing into association a compound provided herein with the carrier and, optionally, at least one accessory ingredient. In general, the formulations are prepared by uniformly and intimately bringing into association a compound provided herein with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound provided herein as an active ingredient. A compound provided herein may also be administered as a bolus, electuary or paste.

In solid dosage forms for oral administration (capsules, tablets, pills, dragees, powders, granules and the like), the active ingredient is mixed with at least one pharmaceutically acceptable carrier, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such carriers as lactose or milk sugars, as well as high molecular weight polyethylene glycols and the like.

A tablet may be made by compression or molding, optionally with at least one accessory ingredient. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions provided herein, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally comprise opacifying agents and may be of a composition that they release the active ingredient(s) only, or, in a certain portion of the gastrointestinal tract, optionally, in a delayed manner. Examples of embedding compositions that can be used include polymeric substances and waxes. The active ingredient can also be in micro-encapsulated form, if appropriate, with at least one of the above-described excipients.

Liquid dosage forms for oral administration of the compounds include pharmaceutically acceptable emulsions, microemulsions, solutions, suspensions, syrups and elixirs. In addition to the active ingredient, the liquid dosage forms may comprise inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may comprise suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions provided herein for rectal or vaginal administration may be presented as a suppository, which may be prepared by mixing at least one compound provided herein with at least one suitable nonirritating excipient or carrier comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations provided herein which are suitable for vaginal administration also include pessaries, tampons, creams, gels, pastes, foams or spray formulations comprising such carriers as are known in the art to be appropriate.

Dosage forms for the topical or transdermal administration of a compound provided herein include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may comprise, in addition to an active compound provided herein, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can comprise, in addition to a compound provided herein, excipients such as lactose, talc, silicic acid, aluminum hydroxide, calcium silicates and polyamide powder, or mixtures of these substances. Sprays can additionally comprise customary propellants, such as chlorofluorohydrocarbons and volatile unsubstituted hydrocarbons, such as butane and propane.

Transdermal patches have the added advantage of providing controlled delivery of a compound provided herein to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also provided herein.

Pharmaceutical compositions provided herein suitable for parenteral administration comprise at least one compound provided herein in combination with at least one pharmaceutically-acceptable sterile isotonic aqueous or nonaqueous solution, dispersion, suspension or emulsion, or sterile powder which may be reconstituted into a sterile injectable solution or dispersion just prior to use, which may comprise sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also comprise adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

Drug-eluting forms include coated or medicated stents and implantable devices. Drug-eluting stents and other devices may be coated with a compound or pharmaceutical preparation and may further comprise a polymer designed for time-release.

In certain embodiments, a compound or pharmaceutical preparation is administered orally. In other embodiments, the compound or pharmaceutical preparation is administered intravenously. In certain embodiments, a compound is attached via a cleavable linker to a solid support that is administered with a catheter. Alternative routes of administration include sublingual, intramuscular, and transdermal administrations.

When the compounds provided herein are administered as pharmaceuticals, to humans and animals, they can be given per se or as a pharmaceutical composition comprising, for example, 0.1% to 99.5%, or 0.5% to 90%, of active ingredient in combination with a pharmaceutically acceptable carrier.

The preparations provided herein may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. In an embodiment, oral administration can be used.

These compounds may be administered to humans and other animals for therapy by any suitable route of administration, including orally, nasally, as by, for example, an aerosol, a spray, rectally, intravaginally, parenterally, intracisternally and topically, as by powders, ointments or drops, including buccally and sublingually.

Regardless of the route of administration selected, the compounds provided herein, which may be used in a suitable hydrated form, and/or the pharmaceutical compositions provided herein, are formulated into pharmaceutically-acceptable dosage forms by conventional methods known to those of skill in the art.

Actual dosage levels of the active ingredients in the pharmaceutical compositions provided herein may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound provided herein employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds provided herein employed in the pharmaceutical composition at levels lower than that required to achieve the desired therapeutic effect and then gradually increasing the dosage until the desired effect is achieved.

In some embodiments, a compound or pharmaceutical composition provided herein is provided to a subject chronically. Chronic treatments include any form of repeated administration for an extended period of time, such as repeated administrations for at least one month, between a month and a year, at least one year, or longer. In many embodiments, a chronic treatment involves administering a compound or pharmaceutical composition provided herein repeatedly over the life of the subject. A chronic treatments can involve regular administrations, for example, at least once a day, at least once week, or at least once a month. In general, a suitable dose such as a daily dose of a compound provided herein will be that amount of the compound that is the lowest dose effective to produce a therapeutic effect. Such an effective dose will generally depend upon the factors described above. Generally doses of the compounds provided herein for a patient, when used for the indicated effects, will range from about 0.0001 to about 100 mg per kg of body weight per day. In an embodiment, the daily dosage will range from 0.001 to 50 mg of compound per kg of body weight, and even more, for example, from 0.01 to 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six, or more sub-doses administered separately at appropriate intervals throughout the day, optionally, in unit dosage forms.

While it is possible for a compound provided herein to be administered alone, in an embodiment, the compound is administered as a pharmaceutical formulation (composition) as described above.

The compounds provided herein may be formulated for administration in any convenient way for use in human or veterinary medicine, by analogy with other pharmaceuticals.

### 6. EXAMPLES

### 6.1. Example 1. Design of Shh-Targeting Cyclic Peptides

Crystallographic studies have provided insights into the structure of Shh in complex with Hedgehog-Interacting Protein (HHIP), a membrane protein that acts as a negative regulator of the Hh pathway (FIG. 2) (Chuang and McMahon 1999). In this complex, HHIP is found to interact with Shh primarily via an extended loop (L2) located in the extracellular domain of HHIP. (Bishop, Aricescu et al. 2009; Bosanac, Maun et al. 2009) Based on this information, the inventors recognized that a cyclic peptide encompassing the HHIP L2 loop sequence would provide a viable starting point for the development of an agent capable of disrupting the Shh/Patched protein-protein interaction. Specifically, the inventors recognized that, starting from a 13-residue peptide sequence spanning residues 375 to 387 of HHIP, a cyclic peptide mimic of HHIP L2 could be obtained by bridging the positions occupied by Met5 (corresponding to Met379 in HHIP) and Leu11 (corresponding Leu385 in HHIP) or, alternatively, by bridging the positions occupied by Met8 (corresponding to Met382 in HHIP) and LeuI1 (corresponding Leu385 in HHIP). To this end, a suitable linker was identified, which corresponds to a thioether-based linker generated via a nucleophilic substitution reaction between a cysteine residue and the cysteine-reactive amino acid O-2-bromoethyl-tyrosine (O2beY). (Bionda, Cryan et al. 2014) The introduction of the inter-side-chain linkage was expected to reduce the conformational flexibility of the cyclic peptides compared to a L2-derived linear peptide, resulting in enhanced binding affinity toward Shh due to the reduced entropic costs upon binding of the peptide to the protein. A molecular model of the i/i+6 thioether-bridged cyclic peptide, called HL2-m1, was generated and docked into the structure of Shh. These analyses supported the design by showing a good overlap between the backbone of the modeled cyclic peptide and that of HHIP L2 loop in the Shh-bound structure (FIG. 3). Similar conclusions could be drawn for the i/i+3 thioether-bridged cyclic peptide, called HL2-2m1.

To assess the Shh binding properties of the candidate inhibitor HL2-m1, this cyclic peptide was produced as fused to a FLAG tag sequence by recombinant methods. To this end, a gene encoding for a 13mer peptide sequence spanning the HHIP L2 loop (HHIP₃₇₅₋₃₈₇) was cloned into a pET-based expression vector. The codon corresponding to Met5 residue in the HL2-m1 sequence (Met375 in HHIP) was mutated to an amber stop codon (TAG) to allow for the site-selective incorporation of O2beY via amber stop codon suppression. (Liu and Schultz 2010) Residue Leu11 (Leu385 in HHIP) was mutated to cysteine to enable the formation of the desired thioether bridge. The HL2-m1 encoding sequence was then fused, via a GlySerGlySer linker (SEQ ID. NO. 72), to an N-terminal FLAG tag for detection purposes. The HL2-m1 encoding sequence was also fused to a C-terminal GyrA intein (Smith, Vitali et al. 2011) containing a poly-histidine tag to facilitate purification and isolation. The resulting polypeptide construct (FLAG-HL2m-GyrA-H₆) was expressed in E. coli cells in the presence of O2beY and a O2beY-specific orthogonal aminoacyl-tRNA synthetase/tRNA pair. (Bionda, Cryan et al. 2014) After protein purification via Ni-affinity chromatography, the FLAG-tagged HL2-m1 peptide (corresponding to FLAG-HL2-m in Table 1) was cleaved from the GyrA intein with thiophenol and purified by HPLC. The identity of the cyclic peptide was confirmed by MS analysis (calc. m/z for [M+H]⁺: 3039.2 Da; obs: 3039.8 Da). Using a similar strategy, two additional cyclic peptides were prepared, namely (i) a HL2-m1 analog in which the orientation of the linker is reversed compared to HL2-m1 (i.e., FLAG-HL2-mlr, Table 1) and (ii) the i/i+3 thioether-bridged cyclic peptide corresponding to FLAG-HL2-2m in Table 3. In addition, a reference compound corresponding to a linear L2-derived peptide sequence (FLAG-HL2-pep: MDYKDDDDK-GSGS-TLDDMEEMDGLSD) (SEQ ID NO: 63) was prepared for comparison purposes.

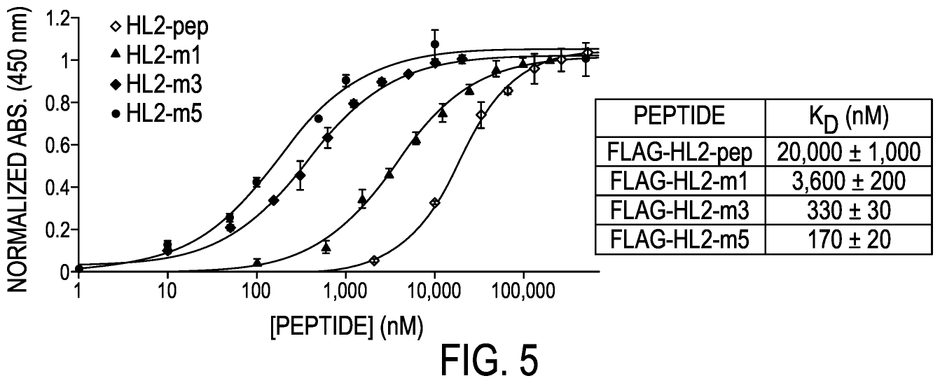

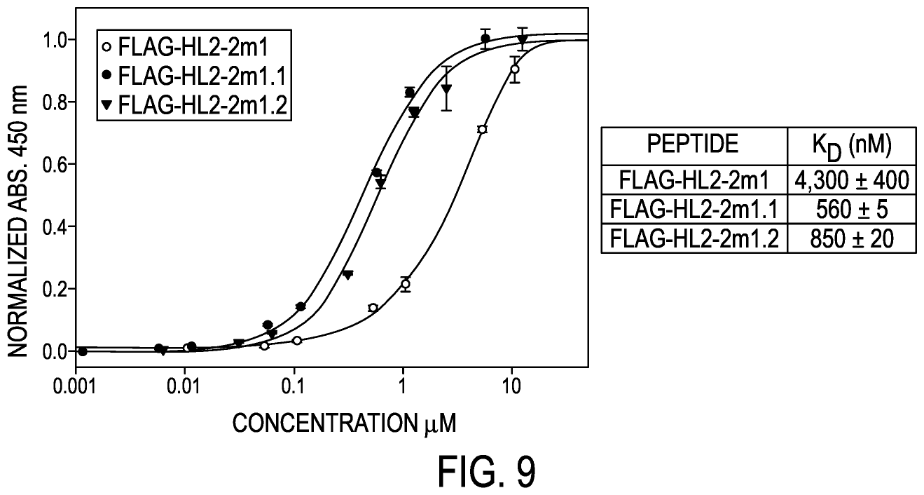

To measure the binding affinity of the peptides for Shh, an in vitro assay was established in which recombinant GST-fused Shh is immobilized on microtiter plates and then exposed to the FLAG-tagged peptide at varying concentrations. The Shh-bound peptide is then quantified colorimetrically (λ₄₅₀) using a horseradish peroxidase (HRP)-conjugated anti-FLAG antibody. Using this assay, the FLAG-HL2-m1 and FLAG-HL2-2m1 peptides were determined to bind Shh with a K_{D} of 3.6 FM and 4.3 μM, respectively (FIGS. 5 and 9). In comparison, the linear L2-derived peptide (FLAG-HL2-pep) binds Shh with significantly reduced affinity (K_{D}=20 μM). These results demonstrated the functionality and superior performance of the cyclic peptides as Shh targeting agents compared to a linear L2-derived peptide. Furthermore, reversal of the orientation of the O2beY/Cys pair (i.e., FLAG-HL2-mlr vs. FLAG-HL2-m1) resulted in cyclic peptides with comparable Shh binding affinity, indicating that variations in the inter-side-chain crosslink are tolerated.

Experimental Procedures.

Cloning, expression, and purification of GST-Hh proteins. Genes encoding for human Shh, Ihh, and Dhh were amplified by PCR and cloned into the Nco I/Xho I cassette of the expression vector pET42b (Novagen). This process resulted in the C-terminal fusion of the Hh protein sequence to that of glutathione-S-transferase (GST) protein containing a poly-histidine tag. The GST-Hh fusion proteins were expressed in BL21(DE3) cells by growing recombinant cells in LB medium with kanamycin (30 μg/mL). At an OD₆₀₀ of 0.6, cells were induced with IPTG (1 mM) and grown for 20 hours at 27° C. The proteins were purified by Ni-NTA chromatography (Invitrogen) according to the manufacturer's instructions. After elution, the proteins were buffer exchanged with PBS buffer (10 mM Na₂HPO₄, 1.8 mM KH₂PO₄, 137 mM NaCl, 2.7 mM KCl, pH 7.4) and stored at −80° C. The identity and purity of the purified proteins were confirmed by DNA sequencing and SDS-PAGE gel electrophoresis.

Cloning of FLAG-HL2-m1, FLAG-HL2-mlr, and FLAG-HL2-pep constructs. A DNA sequence encoding for the HL2-m1 sequence N-terminally fused to a FLAG tag sequence and C-terminally fused to M. xenopi GyrA intein (MDYKDDDDK-GSGS-TLDD(amber stop)EEMDGCSD-GyrA) (SEQ ID NO: 64) was assembled by PCR. The resulting gene was cloned into the BamH I/Xho I cassette of the expression vector pET22b (Novagen), resulting in the fusion of poly-histidine (H₆) tag to the C-terminus of the intein. Using a similar procedure, plasmid vectors for the expression of FLAG-HL2-mlr-GyrA (MDYKDDDDK-GSGS-TLDDCEEMDG(amber stop)SD-GyrA) (SEQ ID NO: 65) and FLAG-HL2-pep-GyrA (MDYKDDDDK-GSGS-TLDDMEEMDGLSD-GyrA) (SEQ ID NO: 66) constructs were prepared.

Biosynthesis and purification of macrocyclic peptides. The plasmids encoding for the GyrA-fused peptides were co-transformed into E. coli BL21(DE3) cells harboring a pEVOL_O2beY-RS vector (Bionda, Cryan et al. 2014) for the co-expression of an orthogonal O2beY-RS/tRNAcuA pair for amber stop codon suppression with O2beY. The corresponding protein products were expressed in 1.0 L M9 media containing ampicillin (100 μg/mL), chloramphenicol (34 μg/mL), and O2beY (2 mM) by induction with arabinose (0.06% m/v) and IPTG (1 mM). The GyrA-fused peptides were purified by Ni-NTA chromatography (Invitrogen) and the eluted proteins were buffer exchanged with potassium phosphate buffer (10 mM potassium phosphate, 150 mM NaCl, pH 7.5). Cleavage of the intein was carried out in the presence of 20 mM TCEP and 10 mM thiophenol. The cleaved peptides were purified by fractionation via solid phase extraction, followed by further purification by reverse-phase HPLC using a Grace C18 column (120 Å; 250×10 mm) and a 5->95% gradient of acetonitrile in water (+0.1% TFA). The peptide identity was confirmed by MALDI-TOF mass spectrometry and the concentration was determined by HPLC (OD₂₂₀) using a calibration curve generated with a reference peptide of identical length. Typical yields for the recombinantly produced cyclic peptides obtained using this procedure was between 0.5 and 1.5 mg/L culture.

Hh protein binding assay. Shh binding activity/affinity of the linear and cyclic peptides was measured using the immunoassay outlined in FIG. 4. For these experiments, GST-Shh was immobilized on 96-well microtiter plates (100 μL of 4 μM GST-Shh solution in PBS buffer), followed by blocking with bovine serum albumin. After washing, each well was incubated with 100 μL of purified FLAG-fused peptide at varying concentrations for 1 hour at room temperature. After washing, each well was added with 100 μL of 1:2500 dilution of HRP-conjugate mouse anti-FLAG polyclonal antibody (Sigma-Aldrich) for 1 hour at room temperature. After washing, 100 μL of 2.2 mM o-phenylenediamine dihydrochloride, 4.2 mM urea hydrogen peroxide, 100 mM dibasic sodium phosphate and 50 mM sodium citrate, pH 5.0 was added to each well, followed by measurement of the absorbance at 450 nm using a Tecan Infinite 1000 plate reader. Equilibrium dissociation constants (K_{D}) were determined by fitting the dose-response curves (FIG. 5) to a 1:1 binding isotherm equation via non-linear regression using SigmaPlot.

### 6.2. Example 2. Affinity Maturation of Cyclic Peptides

The cyclic peptide HL2-m1 was selected as the parent molecule for the development of cyclic peptide inhibitors with further improved Shh binding affinity. To this end, we took advantage of the capability of producing this cyclic peptide, and sequence-permutated variants thereof, directly in bacterial cells. The applied strategy is outlined in FIG. 4 and entails the generation of FLAG-HL2-m1 variant libraries in which at least one amino acid position of the cyclic peptide sequence is/are genetically randomized using degenerate codons (e.g., NNK codon). The resulting cyclic peptide libraries are biosynthesized in bacterial cells arrayed on 96-well plates, where each well contains an individual member of the library. The cells are lysed and the released FLAG-tagged peptides are screened in a high throughput manner for their Shh binding activity using the colorimetric immunoassay described in Example 1. Library members with improved Shh binding affinity (‘hits’) are identified by comparing their response in the assay to that of the parent molecule from which the library was prepared (e.g., HL2-m1 sequence) or another reference (cyclic) peptide.

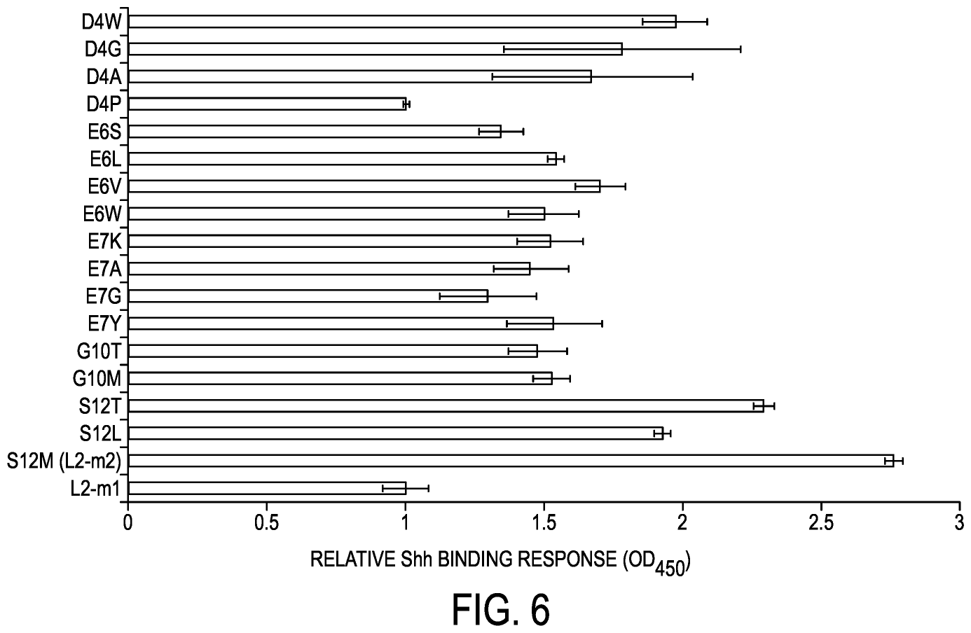

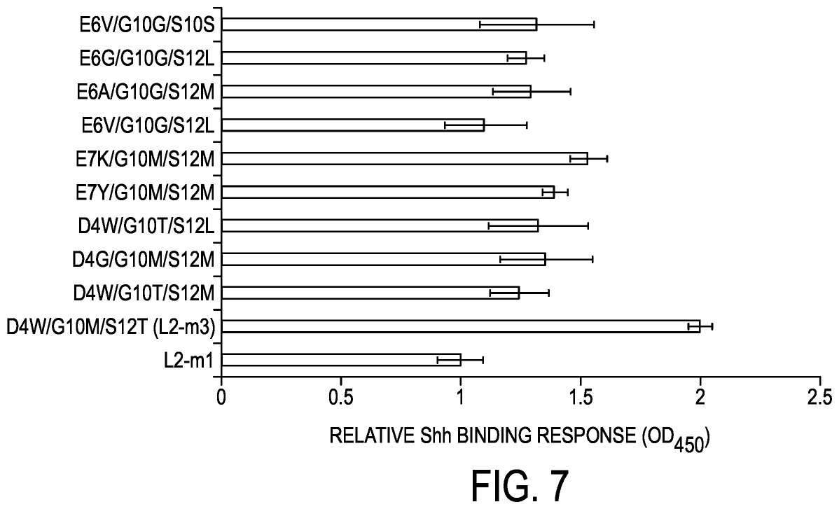

In a first round of affinity maturation, five positions within the parent FLAG-HL2-m1 cyclic peptide (i.e., Asp4, Glu6, Glu7, Gly10, Ser12) were individually randomized by site-saturation mutagenesis using a NNK codon. The resulting cyclic peptide libraries were arrayed on 96-well plates (90 clones/plate), followed by in-cell production of the corresponding cyclic peptide and screening using the in vitro Shh binding assay. From each library, multiple library members were found to exhibit improved Shh binding activity compared to the parent compound FLAG-HL2-m1 (FIG. 6). Among them, a variant containing a Ser12Met mutation, called FLAG-HL2-m2, was selected as the most promising hit. Based on the structure-activity information gathered from these experiments, second-generation libraries were prepared by recombining the most beneficial mutations identified for position 4 (A/D/G/W), 6 (L/S/V/W/E), 7 (K/Y/A/E), 10 (G/M/T), and 12 (L/M/T/S). Upon screening of the resulting libraries (˜500 recombinants) according to the strategy outlined in FIG. 4, several cyclic peptide variants with improved Shh binding activity compared to HL-m1 were identified (FIG. 7). Among them, the cyclic peptide HL2-m3 was selected as the most promising one. By sequencing, the HL2-m3 sequence was determined to contain a total of three mutations, namely D4W, G10M, and S12T, compared to the HL2-m1 sequence (Table 1). Upon expression and purification as stable GyrA-fusion, the FLAG-tagged cyclic peptide was cleaved from the intein with thiophenol. After purification, FLAG-HL2-m1 was determined to bind Shh with a K_{D} of 330 nM (FIG. 5), which corresponds to a 11-fold increase in Shh binding affinity compared to FLAG-HL2-m1.

Next, all of the yet unmodified positions within the HL2-m3 sequence (relative to HL2-m1) were randomized by site-saturation mutagenesis. These positions correspond to residue Thr1, Leu2, Asp3, Glu6, Glu7, Met8, Asp12 of the HL2-m3 sequence. Screening of the resulting libraries (˜650 recombinant clones) revealed the presence of several cyclic peptide variants with Shh binding activity comparable to HL2-m3 or HL2-m1. In addition, two improved HL2-m3-derived variants were identified, which carry a Glu7Ala and a Asp3Ser mutation, respectively. The mutations were combined to yield a further improved Shh-binding cyclic peptide, which was named HL2-m5. HL2-m5 contains a total of five amino acid substitutions compared to HL1-m1 (Table 1). Upon isolation in pure form, FLAG-HL2-m5 was found to bind Shh with a K_{D} of 170 nM (FIG. 5), which corresponds to a nearly 120-fold higher binding affinity for Shh compared to the linear L2-derived peptide (i.e., FLAG-HL2-pep).

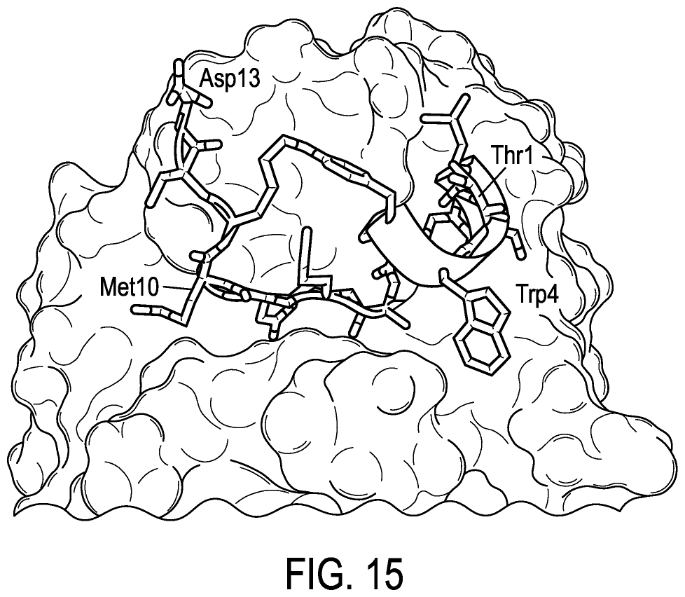

To gain further insights into the role of the beneficial mutations accumulated in HL2-m5, a model of the cyclic peptide in complex with Shh was generated using Rosetta simulations (FIG. 15). Inspection of the complex indicated the occurrence of beneficial interactions between the Trp4 and Met10 residues of HL2-m5 with regions of the Shh surface that are not contacted by the corresponding residues in the L2 loop of HHIP (FIG. 3). Particularly interesting is the role of Trp4, whose aryl ring inserts into a nearby cleft on the Shh surface. Experimentally, the energetic importance of this interaction is corroborated by the identification of a Asp4Trp mutation among the most active compounds isolated from the single site mutagenesis libraries derived from HL2-m1 (FIG. 6). The beneficial effect of the Ala residue in position 7 of cyclic peptide HL2-m5 is supported by the approximately 2-fold increase in Shh binding affinity as a result of Asp7Ala mutation. An identical substitution was also identified as beneficial during screening of the HL2-m1 derived libraries (FIG. 6). Residue 7 is located at the C-terminal end of the two-turn alpha-helix and the beneficial effect of the alanine substitution at this position can be rationalized based on stabilization of an α-helical conformation in this region of the molecule. As observed for the HL2-m1/Shh complex, the side-chain of the Asp9 residue of HL2-m5 in the modeled peptide/Shh complex is found to interact with the Zn(II) ion within the binding cleft of Shh (FIG. 15), thus mimicking the energetically important interaction found between the metal ion and Asp383 residue in HHIP (FIG. 2). (Bishop, Aricescu et al. 2009; Bosanac, Maun et al. 2009)

Using a similar strategy, the HL2-2m1 scaffold was also further optimized via affinity maturation. In this case, a large cyclic peptide library was produced on a phage display format by simultaneous randomization of positions Leu2, Asp3, Met5, and Ser12 via site-saturation mutagenesis. The resulting library (1×10⁶ recombinants) was panned against immobilized Shh followed by elution and amplification of the Shh-bound phages. After three cycles of library enrichments, two optimized sequences were obtained, called HL2-2m1.1 and HL2-2 m1.2 (Table 3). Upon expression and isolation in FLAG tag-fused forms, these cyclic peptides were determined to bind Shh with submicromolar K_{D} (FIG. 9).

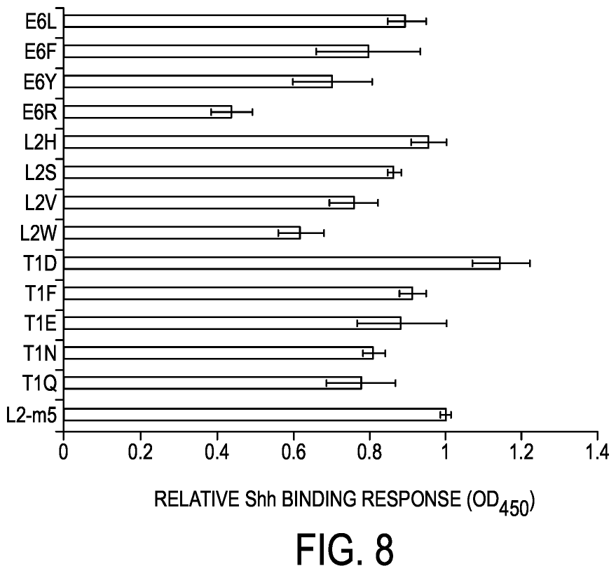

FIG. 8 shows relative Shh binding activity for representative cyclic peptides from HL2-m5-derived site-saturation mutagenesis libraries. Shh binding activity was determined using the colorimetric assay with immobilized GST-Shh protein, FLAG tag-fused peptides, and HRP-conjugated anti-FLAG antibodies. The absorbance values (X axis) are normalized to that of cyclic peptide FLAG-HL2-m5. Indicated mutations (Y axis) are relative to the FLAG-HL2-m5 sequence according to numbering scheme shown in Table 1.

Experimental Procedures.

Library construction. The single-site site-saturation libraries were constructed via overlap extension PCR using pET22_FLAG-(HL2-m)-D-GyrA as the template and the appropriate mutagenizing primers. The PCR product was cloned into the BamH I/Xho I cassette of pET22_FLAG-(HL2-m1)-D-GyrA and the recombinant plasmids transformed in DH5a cells and selected on LB plates containing ampicillin (100 μg/mL). The recombination libraries were prepared in a similar manner but using primers with partially randomized codons (codons: KGB, WAM, KGG, WYG, AYG) to encode for the desired subset of amino acids at each target position. The resulting plasmid libraries were pooled and transformed into cells containing the pEVOL_O2beY-RS vector (Bionda, Cryan et al. 2014) for co-expression of the orthogonal O2beY-RS/tRNA_{CUA} pair. Recombinant cells were selected on LB plates containing ampicillin (100 μg/mL) and chloramphenicol (34 ug/mL) and individual colonies from these plates were used to inoculate 1.0 mL LB media containing the two antibiotics in 96-deep well plates. After overnight growth at 37° C., 50 μL from each well was used to inoculate a replica plate containing 1 mL M9 medium containing ampicillin (100 μg/mL) and chloramphenicol (34 μg/mL). Cells were grown to an OD₆₀₀ of 0.6 in a plate shaker at 37° C., and then induced with arabinose (0.06% m/v) and O2beY (2 mM). After one hour, cells were induced with IPTG (1 mM) and grown at 27° C. for 18-20 hours.

Library screening. The 96-well plate cell cultures were pelleted by centrifugation and then washed once with PBS buffer. Cell pellets were then resuspended in lysis buffer (50 mM potassium phosphate, 150 mM NaCl, 10 mM MgCl₂, 0.8 μg/ml DNase, 0.8 mg/mL lysozyme, pH 7.5) and incubated for 1 hour and 15 minutes at 37° C. After centrifugation, 200 uL of the clarified lysate was used for measuring Shh binding activity using the immunoassay described earlier. Positive hits were identified upon comparison with the reference macrocycle and deconvoluted via DNA sequencing of plasmids extracted from the master plate.

### 6.3. Example 3. Synthesis of Cyclic Peptides

A solid-phase method was developed to access the cyclic peptides by chemical means. This strategy involves the incorporation of a dipeptide building block encompassing the O2beY/Cys thioether crosslink during solid-phase peptide synthesis (SPPS), followed by on-resin cyclization and cleavage/deprotection of the peptide from the resin (FIG. 17). As shown in FIG. 16, the dipeptide building block was prepared in a few steps from commercially available Tyr(OAllyl) (1) and cystine tert-butyl ester (4). The synthetic route for the preparation of HL2-m5 is shown in FIG. 17 and it was described in detail earlier. Using this approach, the cyclic peptide HL2-m1 was obtained in N-acetylated, C-amidated form with an overall yield of 15%. The same protocol was applied to afford the cyclic peptides HL2-m1 and HL2-m3 (Table 1) in similar yields.

Experimental Procedures.

Synthesis of N-Alloc-L-Tyrosine allyl ester (2). L-Tyrosine allyl ester (pToluene sulfonate salt) (1) (1.7 g, 4.32 mmol) was dissolved in 15 mL of water. Sodium carbonate was added to the solution (1,361 g, 12.96 mmol, 3 equiv), then allyl chloroformate (6.48 mmol, 0.68 mL, 1.5 equiv) was added dropwise to the reaction at 0° C. The reaction was stirred for 15 hours, after which it was quenched by addition of 1 M HCl (15 mL) and extracted with ethyl acetate (2×40 mL). The combined organic layers were washed with water (70 mL) and dried over Na₂SO₄. After removal of the solvent by rotary evaporation, the crude product was purified on a silica gel column using hexanes/ethyl acetate from 9:1 to 8:2 as eluent to yield 2 as a colorless oil (0.92 g, 70%). ¹H-NMR (400 MHz, MeOD) δ 6.99-6.97 (d, 2H, J=8.4 Hz), 6.67-6.65 (d, 2H, J=8.4 Hz), 5.88-5.79 (m, 2H, J=6.4 Hz), 5.26-5.09 (m, 4H, J=9.4 Hz), 4.54-4.53 (d, 2H, J=5.6 Hz), 4.45-4.44 (d, 2H, J=4.8 Hz), 4.34 (t, 1H, J=6.0 Hz), 3.01-2.79 (m, 2H, J=8.8 Hz). ¹³C-NMR (100 MHz, MeOD) δ 171.7, 171.4, 156.7, 155.8, 132.7, 131.7, 129.7, 127.3, 117.1, 116.0, 114.7, 65.2, 64.9, 59.9, 55.7, 36.3, 19.4, 12.9 MS-ES: Calc. Mass for C₁₆H₉NO₅: 305.3 Da. Obs. Mass for [M−H]⁻: 304.3 Da.

Synthesis of N-Alloc O-(2-bromoethyl)-L-Tyrosine allyl ester (3). N-Alloc-L-tyrosine allyl ester 2 (0.92 g, 3.02 mmol) was dissolved in 15 mL dry DMF under argon flow. K₂CO₃ (1.25 g, 9.06 mmol, 3 equiv) was added to the reaction and stirred vigorously for 10 minutes. Then 1,2-dibromoethane (0.8 mL, 9.06 mmol, 3 equiv) was added to the suspension dropwise. The reaction was stirred overnight and then quenched with HCl 1M (15 mL). The crude product was extracted using ethyl acetate (2×40 mL). The combined organic layers were washed with brine and dried over Na₂SO₄. After removal of the solvent by rotary evaporation, the crude product was purified on silica gel column using hexanes/ethyl acetate from 9:1 to 7:3 to yield 3 as a colorless oil (0.43 mg, 35%) and recovered starting material (0.55 g, 60%). ¹H-NMR (400 MHz, MeOD) δ 7.02-7.00 (d, 2H, J=8.4 Hz) 6.80-6.78 (d, 2H, J=8.8 Hz), 5.89-5.78 (m, 2H, J=6.0 Hz), 5.29-5.15 (m, 4H, J=10.0 Hz), 4.57-4.56 (d, 2H, J=5.6 Hz), 4.52-4.50 (d, 2H, J=5.2 Hz), 4.57-4.51 (m, 1H,), 4.23-4.20 (t, 2H, J=6.4 Hz), 3.59-3.56 (t, 2H, J=6.4 Hz), 3.08-2.97 (m, 2H, J=6.4 Hz). ¹³C-NMR (100 MHz, MeOD) δ 171.1, 157.1, 155.3, 132.4, 131.3, 130.3, 128.4, 118.9, 117.6, 114.7, 67.7, 65.9, 65.6, 54.7, 37.2, 28.9. ESI-MS: Calc. Mass for C₁₈H₂₂BrNO: 412.28 Da. Obs. Mass for [M+Na]⁺: 434.3 Da.

Synthesis of (2R,2′R)-di-tert-butyl 3,3′-disulfanediylbis(2-((((9H-fluoren-9-yl)methoxy)carbonyl)amino)propanoate) (5). L-Cystine tert-butyl ester 4 (2 mmol, 704 mg) was suspended in 10 mL of THF and N-methyl morpholine (4 mmol, 0.520 mL, 2 equiv) was added to the suspension. The solution was chilled to 0° C. in an ice bath and then 9-fluorenylmethyl-N-succinimidyl carbonate (Fmoc-OSu) (2 mmol, 675 mg) was added slowly portion-wise. The reaction was stirred for 18 hours, allowing it to return at room temperature. The solvent was removed under reduced pressure and the crude product was dissolved in 25 mL of ethyl acetate. The organic layer was washed with 20 mL of HCl 0.1 M and then with 20 m1 of brine. The organic layer was dried over Na₂SO₄ filtered and evaporated. The crude product was purified by silica gel column using hexanes/diethyl ether (7:3) to yield 5 as a white solid (1.2 g, 75%). ¹H-NMR (400 MHz, CDCl₃) 7.76-7.74 (d, 4H, J=7.6 Hz), 7.62-7.60 (d, 4H, J=7.2 Hz), 7.41-7.37 (t, 2H, J=7.2 Hz), 7.32-7.29 (t, 2H, J=7.2 Hz), 4.48-4.46 (m, 2H), 4.39-4.38 (t, 4H, J=7.2 Hz), 4.25-4.23 (t, 2H, J=7.2 Hz), 3.24-3.15 (m, 4H), 1.47 (s, 9H). ¹³C-NMR (100 MHz, CDCl3) 169.4, 155.5, 143.6, 127.5, 126.9, 125.0, 125.0, 119.8, 82.9, 79.8, 67.1, 60.2, 46.9, 28.6, 28.2, 27.8, 27.3. ESI-MS. Calc. Mass for C₄₄H₄₈N₂O₈S₂: 796.29 Da Obs. Mass: 819.4 [M+Na].

Synthesis of N-Fmoc-L-Cysteine t-butyl ester (6). 1.2 g of N,N′-Fmoc-Cystine t-butyl ester (5) (1.72 mmol) was dissolved in 20 mL of THF. Triphenylphosphine (0.9 g, 3.44 mmol, 2 equiv) was added to the solution and the reaction mixture was stirred for 2 hours at room temperature. Water (2 mL) was then added and the reaction mixture was stirred for 10 hours. The solvent was removed by rotary evaporation and the residue was taken up in EtOAc, washed with 10% citric acid and brine, dried over sodium sulfate and concentrated. The crude product was purified on silica gel column using hexanes/ethyl acetate from 95:5 to 8:2 ratio to yield 6 as a colorless oil (0.4 g, 60%). ¹H-NMR (400 MHz, CDCl3) 7.78-7.76 (d, 2H, J=7.6 Hz), 7.62-7.60 (d, 2H, J=7.2 Da), 7.42-7.39 (t, 2H, J=7.6 Da), 7.34-7.30 (t, 2H, J=7.6 Hz), 4.54 (m, 1H), 4.43-4.39 (t, 2H, J=7.2 Hz), 4.25-4.21 (t, 1H, J=6.8 Hz), 3.00-2.98 (m, 2H, J=7.6 Hz), 1.43 (s, 9H). ¹³C-NMR (100 MHz, CDCl3) 171.0, 159.9, 141.1, 127.5, 127.4, 126.9, 125.0, 124.6, 119.8, 82.5, 68.2, 67.1, 60.2, 46.9, 27.9, 27.7, 20.8. MS-ESI⁻: Calc. Mass for C₂₂H₂₅NO₄S: 399.51 Da Obs. Mass: 422.3 [M+Na].

Synthesis of (R)-tert-butyl 2-((((9H-fluoren-9-yl)methoxy)carbonyl)amino)-3-((2-(4-((S)-2-(((allyloxy)carbonyl)amino)-3-oxo-3-(prop-1-en-1-yloxy)propyl)phenoxy)ethyl)thio) propanoate (6b). N-alloc-O-(2-bromoethyl)-L-Tyrosine allyl ester 3 (0.43 mg, 1.04 mmol) and N-Fmoc-L-cysteine t-butyl ester 6 (0.41 mg, 1.04 mmol) were dissolved in 5 mL of dry ethyl acetate. Tetrabutylammonium bromide (1.29 g, 4.0 mmol) was dissolved in 5 mL of nitrogen-sparged NaHCO₃ solution (0.5 M), which was added to the reaction mixture dropwise under argon. The reaction was stirred vigorously for 16 hours, then diluted with ethyl acetate. The organic layer was washed with water and brine, dried over Na₂SO₄ and concentrated. The crude product was purified on silica gel column using hexanes/ethyl acetate from 9:1 to 7:3 to yield 7 as a colorless oil (0.29 g, 40%). ¹H-NMR (400 MHz, CDCl₃) δ 7.73-7.71 (d, 2H, J=7.2 Hz), 7.57-7.55 (d, 2H, J=7.6 Hz), 7.37-7.34 (t, 2H, J=7.6 Hz), 7.28-7.24 (t, 2H, J=8.0 Hz), 6.99-6.96 (d, 2H, J=8.4 Hz), 6.78-6.75 (d, 2H, J=8.4 Hz), 5.82 (m, 2H, J=6.4 Hz), 5.29-5.15 (m, 4H, J=10.8 Hz), 4.57-4.55 (d, 2H, J=5.6 Hz), 4.52-4.50 (d, 2H, J=5.2 Hz), 4.35-4.34 (t, 2H, J=3.6 Hz), 4.18 (t, 1H, J=6.8 Hz), 4.07 (t, 2H, J=7.2 Hz), 3.10-2.99 (m, 4H, J=5.2 Hz), 2.90-2.87 (t, 2H, J=6.0 Hz), 1.45 (s, 9H). ¹³C-NMR (100 MHz, CDCl₃) δ 171.1, 169.5, 157.39, 155.5, 155.3, 143.6, 141.1, 131.2, 130.2, 127.9, 127.5, 126.9, 124.9, 119.8, 118.9, 117.7, 114.5, 82.8, 67.6, 66.9, 65.8, 65.6, 60.2, 54.0, 54.2, 46.9, 37.1, 35.0, 31.7, 27.8, 14.0.

Synthesis of (R)-2-((((9H-fluoren-9-yl)methoxy)carbonyl)amino)-3-((2-(4-((S)-2-(((allyloxy)carbonyl)amino)-3-oxo-3-(prop-1-en-1-yloxy)propyl)phenoxy)ethyl)thio) propanoic acid (7). To a solution of 6b (0.29 g, 0.4 mmol) in CH₂Cl₂ (6 mL) was added 4 mL of trifluoroacetic acid (TFA) at 0° C. The reaction was stirred at 0° C. for 2 hours. The product was concentrated in vacuo, then washed extensively with diethyl ether. The final product was yielded as a white crystalline powder. ¹H-NMR (400 MHz, MeOD) δ 7.77-7.75 (d, 2H, J=7.2 Hz), 7.65-7.63 (d, 2H, J=6.8 Hz), 7.37-7.34 m (t, 2H, J=7.6 Hz), 7.29-7.25 (t, 2H, J=7.6 Hz), 7.08-7.057 (d, 2H, J=8.4 Hz), 6.82-6.79 (d, 2H, J=8.8 Hz), 5.85 (m, 2H), 5.29-5.11 (m, 4H), 4.57-4.30 (m, 8H, J=5.6 Hz), 4.19 (t, 1H), 4.11-4.08 (t, 2H, J=6.0 Hz), 2.99-2.84 (m, 6H). ¹³C-NMR (100 MHz, MeOD) δ 173.5, 171.5, 171.4, 169.8, 157.6, 157.4, 156.1, 156.0, 155.8, 143.7, 141.3, 132.4, 131.7, 131.3, 130.4, 128.1, 128.0, 127.7, 127.1, 125.1, 124.8, 120.0, 119.2, 119.1, 118.3, 114.8, 114.7, 83.2, 67.9, 67.3, 66.2, 55.9, 55.0, 47.1, 37.4, 35.9, 31.9.

Solid-phase peptide synthesis. The macrocyclic peptides were manually synthesized via standard solid-phase Fmoc chemistry using MBHA rink amide resin (loading: 0.25 mmol/g) in a polypropylene reaction vessel. Standard Fmoc-protected amino acids were used as building blocks, with the exception of Asp10, for which N-Fmoc-Asp(OEpe)CO₂H was used. Loading of the first amino acid and subsequent elongation steps were carried out using 5 equiv Fmoc-protected amino acid preactivated with COMU (4.95 equiv) and DIPEA (10 equiv) in DMF for 1 hour at room temperature. The Fmoc protecting group was removed with 30% piperidine in DMF (2×10 min). To introduce the dipeptide building block, compound 7 (75 mg 0.11 mmol) was pre-activated with COMU/DIPEA in DMF and added to the resin for one hour at room temperature. Prior to the cyclization reaction, deprotection of the Alloc/allyl groups was carried out using Pd(PPh₃)₄ (1 equiv)/PhSiH₃ (20 equiv) in dry DCM (2×45 minutes). Peptide cyclization was carried out at millimolar pseudo-dilution using a mixture of PyBOB (2 equiv), HOBt (2 equiv), and DIPEA (4 equiv) in DMF, for two cycles of 12 hours. After addition of the last amino acid in the sequence, the resin-bound peptide was acetylated by two treatments with a mixture of acetic anhydride (0.5 M), DIPEA (0.015 M, and HOBt (0.125 M) in DMF for 10 minutes. The peptides were cleaved from the solid support using a solution of TFA:H₂O:TIS (95:2.5:2.5 v/v/v) for 3 hours at room temperature. After removal of the resin by filtration, the crude peptide was precipitated with cold MTBE, re-dissolved in 1:1 water/acetonitrile solution, and lyophilized. The crude peptide was purified by reverse-phase HPLC using an Agilent 1200 system equipped with a Grace C18 column (10 μm; 90 \acute{Å}; 250×10 mm) at a flow rate of 2.5 mL/min and a linear gradient starting from 20% to 80% acetonitrile in water (+0.1% TFA) over 25 min. The purity and identity of all peptide was confirmed by analytical HPLC and LC-MS.

| | | Observed Mass | |
|---|---|---|---|
| Peptide | Calc. Mass | [M + H]⁺ | Retention Time |
| HL2-pep | 1511.59 Da | 1512.44 Da | 9.9 min |
| HL2-m1 | 1559.59 Da | 1559.70 Da | 11.3 min |
| HL2-m3 | 1718.89 Da | 1719.9 Da | 13.2 min |
| HL2-m5 | 1632.84 Da | 1633.82 Da | 13.42 min |

### 6.4. Example 4. Synthesis of Additional Cyclic Peptides

Our finding that the linker in the cyclic peptide L2 mimics can be varied while preserving the ability of the cyclic peptides to bind tightly to Shh (see comparison between FLAG-HL2-m1 vs. FLAG-HL2-2 m1 in Example 1) prompted us to investigate alternative linkers and peptide cyclization strategies for the purpose of developing inhibitors of Hedgehog proteins. To this end, a panel of cyclic peptides were prepared based on the optimized amino acid sequence of HL2-m5, in which the i/i+6 bridge connecting residue 5 and 11 was produced via alkylation of two cysteine residues with a bifunctional alkylating reagent. As described in FIG. 18, these peptides were prepared by assembling the linear peptide via solid-phase peptide synthesis, followed by cyclization in solution. As described earlier, an alternative synthetic protocol involving on-resin cyclization of the peptide was also developed (FIG. 19).

Using this strategy and different cysteine crosslinking reagents (e.g., diiodomethane, 1,3-dibromopropane, o,o-dibromo-xylene, etc.), a panel of cyclic peptides incorporating the optimized HL2-m5 peptide sequence but containing variable inter-side-chain linkages between position 5and 11 was obtained (i.e., m5-s2 through m5-s1, Table 2). In addition, a disulfide-bridged peptide (m5-s, Table 2) was generated by oxidative cyclization of the linear, cysteine-containing peptide in the presence of air. After purification, the peptides were purified by HPLC and their identity confirmed by LC-MS.

| COMPOUND | CROSSLINKING REAGENT | MW | OBS. MASS |
|---|---|---|---|
| M5-S1 | disulfide bridge | 1544.75 | 1568.30 |
| | | | (M + Na) |
| M5-S2 | CH₂I₂ | 1558.78 | 1582.35 |
| | | | (M + Na) |
| M5-S3 | 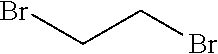 | 1572.80 | 1595.97 (M + Na) |
| M5-S4 | 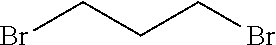 | 1586 | 1611.30 (M + Na) |
| M5-S5 | 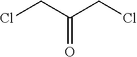 | 1600.81 | 1625.25 (M + Na) |
| M5-S6 | 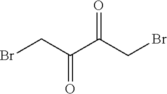 | 1628.82 | 1652.71 (M + Na) |
| M5-S7 | 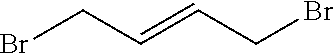 | 1598.84 | 1621.29 (M + Na) |
| M5-S8 | 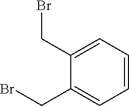 | 1648.9 | 1672.55 (M + Na) |
| M5-S9 | 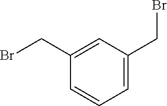 | 1648.9 | 1672.73 (M + Na) |
| M5-S10 | 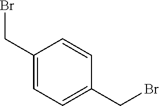 | 1648.9 | 1672.07 (M + Na) |
| M5-S11 | 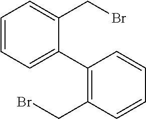 [SMILES: BrCc1ccccc1-c1ccccc1CBr] | 1726.95 | 1750.1 (M + Na) |

Experimental Procedures.

In solution cyclization method. The linear precursor peptide corresponding to TLSWCEAMDMCTD (SEQ ID. NO. 70) was assembled via standard Fmoc-chemistry and solid-phase peptide using Rink amide at a peptide loading of 0.59 mmol/g. In this protocol, Trt-protected cysteines were used as building blocks. After automated synthesis of the peptide, the N-terminal amino group was Fmoc-deprotected with piperidine and acetylated as described earlier. The peptide was cleaved from the resin using a solution of TFA/H₂O/TIS (95:2.5:2.5 v/v/v) and precipitated in cold diethyl ether. For the cyclization reaction, 0.02 mmol of the crude peptide was dissolved in H₂O/THF (9:1), followed by addition of TCEP (2 equiv) and K₂CO₃ (3 equiv). Then, the crosslinking reagent (3 equiv) and TEA (6 equiv) were added to the solution. The reaction was monitored by HPLC and MALDI-TOF MS. Upon completion of the cyclization reaction, the cyclic peptide was purified by HPLC.

On-resin cyclization method. The linear precursor peptide corresponding to TLSWCEAMDMCTD (SEQ ID. NO. 70) was assembled as described above, with the difference that Mtt-protected cysteines were used as building blocks. After the N-acetylation step, the Mmt protecting groups were removed exposing the resin to a solution of 1% TFA in dichloromethane. For the cyclization step, 0.02 mmol of the resin-bound peptide was diluted in 5 mL NMP and the suspension was added with 3 equiv of the crosslinking reagents and 3 equiv of DIPEA. After cyclization was complete, the peptide was cleaved from the resin and purified by RP-HPLC.

### 6.5. Example 5. Shh Binding Activity of Cysteine-Crosslinked Peptides

For assessing the Shh binding affinity of the cysteine-crosslinked peptides described in Example 4, biotinylated variants of these peptides were prepared by introducing a biotin moiety to the N-terminal peptide of the peptide followed by a tetrapeptide (Gly-Ser-Gly-Ser) (SEQ ID NO: 72) spacer (i.e., peptides Biot-m5-s1 through Biot-m5-s10). The biotinylated peptides were tested in a modified variant of the Shh binding assay described in Example 1, where HRP-conjugated streptavidin is used instead of the HRP-conjugated anti-FLAG antibody. As shown by the data reported in the table below, many of the cyclic peptides were found to be able to bind Shh with low micromolar affinity.

| | | OBSERVED | |
|---|---|---|---|
| COMPOUND | MW | MASS | K_{D} (SHH) |
| BIOT-M5-S1 | 2017.26 | 2043.45 (M + Na) | 1.7 μM |
| BIOT-M5-S2 | 2031.29 | 2056.48 (M + Na) | 3.4 μM |
| BIOT-M5-S3 | 2045.31 | 2068.47 (M + Na) | 5.9 μM |
| BIOT-M5-S4 | 2059.34 | 2082.53 (M + Na) | 6.4 μM |
| BIOT-M5-S5 | 2071.35 | 2094.74 (M + Na) | 1.1 μM |
| BIOT-M5-S6 | 2121.41 | 2145.37 (M + Na) | >50 μM |
| BIOT-M5-S7 | 2121.41 | 2144.81 (M + Na) | 9.2 μM |
| BIOT-M5-S8 | 2121.41 | 2144.95 (M + Na) | >50 μM |

The amino acid sequence of cyclic peptide m5-s5 was further modified at the level of residue 10, which was identified as a promising site for improving contacts between the cyclic peptide and the Hedgehog protein (FIG. 15). Accordingly, a Nleu and a N(butyl)glycine were incorporated at this position to yield Biotm5-s7.2 and Biotm5-s7.2-3 (Table 2). To further examine the effect of the inter-side-chain linkage, other two m5-s5-based derivatives were prepared in which the Cys residue at position 11 was replaced with DCys and homocysteine (Biotm5-s7.4 and Biotm5-s7.2-5; Table 2). These cyclic peptides were found to be viable Shh inhibitors exhibiting low micromolar to submicromolar affinity for binding to Shh.

### 6.6. Example 6. Suppression of Hedgehog Pathway Activation in Cells

Having demonstrated the ability of the cyclic peptides such as HL2-m5 to target Shh in vitro, we next examined the activity of this representative compound toward disrupting Shh-mediated Hedgehog pathway signaling in cells. To this end, we utilized a cell-based luciferase reporter assay, (Chen, Taipale et al. 2002) in which mouse embryo fibroblasts (NIH3T3) are transfected with vectors encoding for a Firefly luciferase (FF) gene under a Gli-controlled promoter and a Renilla luciferase (Ren) gene under a constitutive promoter. Hedgehog pathway suppression is measured based on the decrease in Firefly/Renilla luminescence ratio in the presence of the inhibitor. In preliminary experiments, this assay was validated using the Smo inhibitor cyclopamine, which caused full inhibition of Shh-induced luminescence in the cells at a concentration of 10 μM, in accordance with previous reports. (Chen, Taipale et al. 2002)

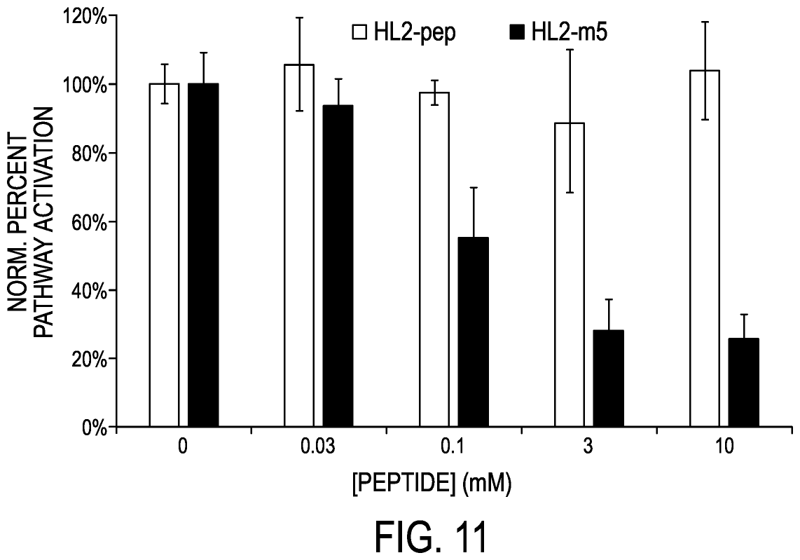

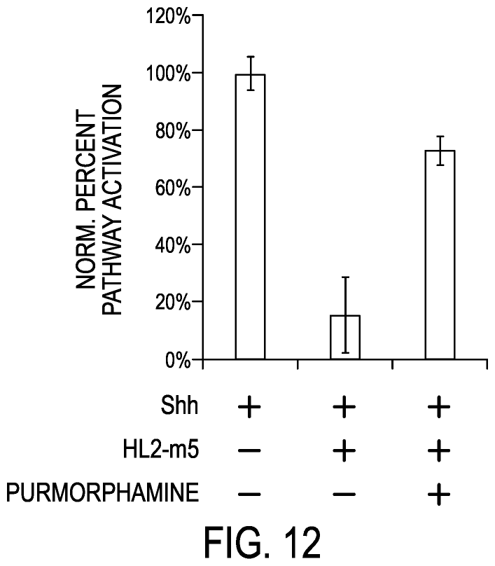

After transfection with the luciferase reporter plasmids, NIH3T3 cells showed strong luminescence in the presence of recombinant N-palmitoylated Sonic Hedgehog (Shh-N) and low luminescence in the absence of Shh-N, thereby confirming Shh-dependent activation of the Hedgehog pathway in the cells. Upon incubation of Shh-N-stimulated cells with HL-m5, a dose-dependent suppression of the luminescence signal was observed (FIG. 11) from which a half-maximal inhibitory concentration (IC₅₀) of 250 nM was determined. In contrast, the linear L2-derived peptide (HL2-pep) showed no inhibitory activity at concentrations up to 30 μM under identical conditions. Incubation of HL-m5-treated cells with purmorphamine, a Smo agonist, (Sinha and Chen 2006) restored activation of the signaling pathway (FIG. 12), thus demonstrating that HL2-m5-dependent inhibition occurs the level of Shh/Patched interaction. Notably, the inhibitory activity of HL2-m5 toward blocking Hedgehog pathway activation in cells is nearly two orders of magnitude higher than that of robotnikinin (IC₅₀˜15 μM), (Stanton, Peng et al. 2009) as determined using a similar cell-based assay. In addition, the IC₅₀ value exhibited by HL2-m5 in the cell-based assay (250 nM) is very similar to the K_{D} value of this compound measured with the in vitro Shh binding assay (170 nM), indicating that the macrocyclic peptide targets Shh with high affinity and specificity even in the presence of cells and complex growth medium.

Gli-reporter Assay. NIH-3T3 cells were passaged twice and then plated in 24-well culture dishes at 5×10⁵ cells/well in DMEM containing 10% FBS and 1% penicillin/streptomycin. After 24 hours, the cells were transfected (TransIT-2020) with a mixture of a firefly luciferase reporter construct under the control of a Gli1 inducible promoter and a Renilla luciferase reporter construct under a constitutive promoter (40:1) (Cignal GLI Reporter Luciferase Kit, Qiagen). Cells were allowed to reach confluency, at which point the media was changed to Opti-MEM containing 1% FBS and added with 4 nM Shh-N(R&D Systems, Minneapolis, Minn.) in sterile PBS buffer. Synthetic HL2-lin and HL2-m5 were added at the same time at varying concentration (0.01-30 uM), and control cells were prepared by adding vehicle only (1% DMSO). Purmorphamine-treated cells were prepared by adding 5 μM purmorphamine to wells containing 10 μM HL2-m5. After growth for 24 hours at 37° C. in a humidified chamber, the cells were harvested and analyzed for Firefly and Renilla luciferase activity using a Tecan Spark-20 plate reader and a DLR kit (Promega) according to the manufacturer's instructions. Luminescence values were normalized to those of the Shh pathway activated control cells. Mean values and standard deviations were calculated from experiments performed at least in duplicate.

### 6.7. Example 7. Suppression of Hedgehog-Dependent Transcriptional Activity

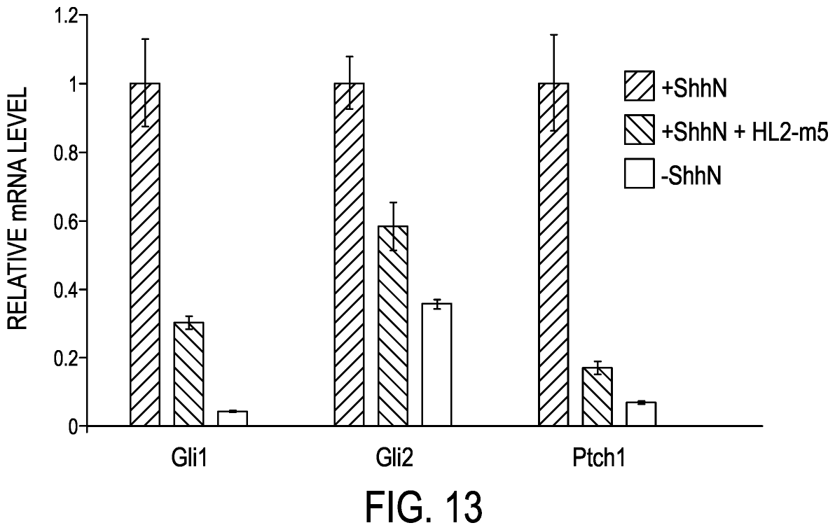

To further validate HL2-m5 as a Hedgehog pathway antagonist, the effect of this compound on the transcriptional activity of two canonical target genes of the pathway, Gi and Ptch1 (FIG. 1) was examined via real-time PCR. As shown in (FIG. 13), a significant reduction (75-85%) of the mRNA levels corresponding to these genes was observed in ShhN-stimulated cells upon incubation with HL2-m5 at 10 μM, relative to compound-untreated cells. Treatment with the macrocyclic peptide also suppresses the mRNA level for the transcription factor Gli2. For both Ptch1 and Gi2, the corresponding transcriptional levels in HL2-m5-treated cells approach those observed in unstimulated cells grown in the absence of Shh-N ligand (FIG. 13). No changes in cell morphology, growth behavior and titer were noted in the presence of HL2-m5, indicating a lack of cytotoxicity at the highest concentration range applied in these experiments. Taken together, the results above demonstrate that the macrocyclic peptide is able to potently inhibit Shh-dependent Hedgehog pathway activation in living cells and suppress signature transcriptional responses resulting from ligand-induced stimulation of the pathway.

Gene transcription analyses. NIH-3T3 cells we passaged twice and plated at a density of 1:3 in DMEM containing 10% FBS and 1% penicillin/streptomycin in 6-well cell culture dishes. Cells were allowed to reach confluency, at which point the media was changed to Opti-MEM containing 1% FBS and added with Shh-N(4 nM). At the same time, the cells were incubated with HL2-m5 (25 uM) or vehicle-only (1% DMSO). After growth for 24 hours at 37° C. in a humidified chamber, the cells were harvested, and total mRNA was collected using TRIazol reagent (ThermoFisher) according to the manufacturer's instructions. cDNA was generated using 1 ug of mRNA using First-Strand RT-PCR with random hexamers (Super-Script First-Strand RT-PCR, ThermoFisher). The relative amounts of Gi, Gi2, and Ptch1 mRNA transcripts were determined by real-time PCR (Bio-Rad CFX thermocycler) using the primers listed in Table S1 and SYBR green TAQ reagent (Bio-Rad) according to the manufacture's protocol. The mRNA levels for the biomarker genes were normalized to that of the reference house-keeping gene cyclophilin. Mean values and standard deviations were calculated from measurements performed in quadruplicate.

### 6.8. Example 8. Hedgehog Analogs

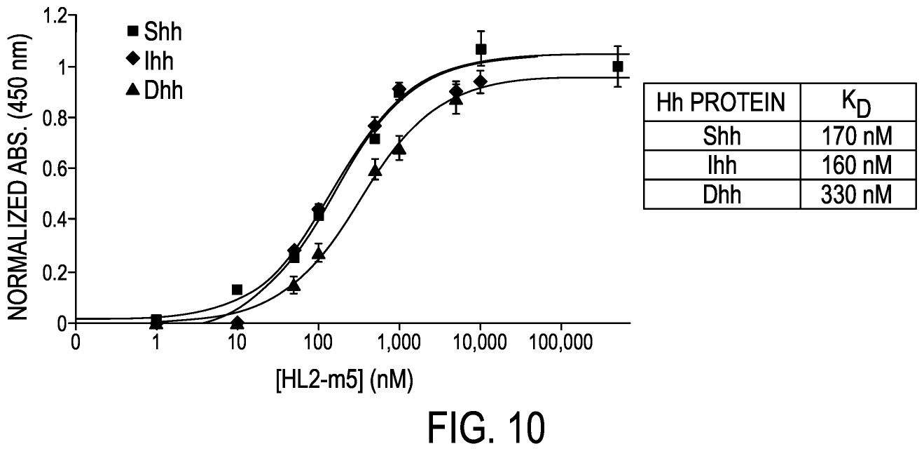

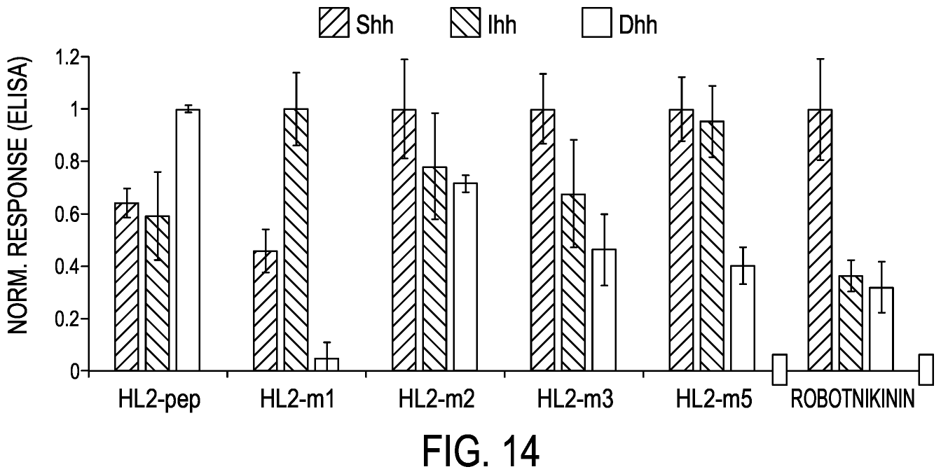

While Shh is the most abundant isoform among Hedgehog proteins, paracrine/autocrine Hedgehog signaling in normal and cancer cells is also mediated by the Indian (Ihh) and Desert (Dhh) isoforms. (Azoulay, Terry et al. 2008; Ibuki, Ghaffari et al. 2013) Hh-targeted inhibitors capable of targeting multiple isoforms of this protein are thus expected to be particularly useful toward suppressing ligand-induced activation of this pathway. Since the Hh isoform selectivity of robotnikinin had not previously investigated, this property was examined by means of a competition assay, whereby inhibition of FLAG-HL2-m5 binding to plate-immobilized GST-Shh, GST-Ihh, or GST-Dhh is measured via the HRP-conjugated anti-FLAG antibody. These experiments showed that robotnikinin has significantly lower affinity toward Ihh and Dhh relative to Shh (FIG. 14). By comparison, HL2-m5 was found to interact with all three analogs of Hh proteins, showing nearly identical activity toward Shh and Ihh (FIG. 14). Consistent with this trend, direct binding experiments showed that HL2-m5 interacts with Ihh and Dhh with a K_{D} of 160 and 330 nM, respectively FIG. 10). Thus, the affinity of HL2-m5 for Ihh and Dhh is nearly identical and only two-fold lower, respectively, than that for Shh (170 nM) as determined using the same assay. These results indicate that the macrocyclic peptide can act as an effective inhibitor for all analogs of Hedgehog protein.

Similar experiments were extended to other representative cyclic peptides, namely HL2-m1, HL2-m2, and HL2-m3 (FIG. 14). The L2-derived linear peptide HL2-pep was found to bind preferentially to Dhh over Shh and Ihh (Dhh>Shh≈Ihh), a selectivity profile that mirrors that of full-length HHIP. (Martinez-Chinchilla and Riobo 2008) On the other hand, the cyclic peptides were found to bind to the three homologs of Hedgehog with varying degree of selectivity. Altogether, these results showed that the cyclic peptides offer promising Hedgehog-targeting compounds that can exhibit a Hedgehog analog selectivity profile that can be both similar and distinct from that of a L2-derived linear peptide or HHIP itself.

Hedgehog analog selectivity. K_{D} values for FLAG-HL2-m5 binding to Ihh and Dhh were determined using the colorimetric immunoassay described earlier but using GST-Ihh and GST-Dhh-coated plates, respectively. The peptide relative binding affinity for the three isoforms of Hedgehog was determined using the same assay and peptide solutions at a fixed concentration of 0.5-1 μM. In this case, binding responses were subtracted against the blank (no peptide sample) and normalized to the highest value measured across the three Hh isoforms. Mean values and standard deviations were calculated from experiments performed at least in triplicate.

Competition assay. A PBS solution (100 uL) containing 10 μM robotnikinin and 400 nM FLAG-HL2-m5 was added to GST-Shh-, GST-Ihh-, and GST-Dhh-coated wells in a microtiter plate. The plates were then treated and developed as described above. The relative affinity of robotnikinin for the three Hh isoforms was expressed as follows: (1−% inhibition)_{G}ST-Hh/(1−% inhibition)_{G}ST-Sₕₕ, where % inhibition in the presence of GST-Shh was 34%. Mean values and standard deviations were calculated from experiments performed in triplicate.

### 6.9. Example 9. Warhead-Containing Cyclic Peptides

Sulfonylfluoride groups have proven to be useful chemical ‘warheads’ for the development of irreversible inhibitors of proteins (see e.g., Narayanan et al, Chem. Sci., 2015, 6, 2650). Cyclic peptide incorporating a sulfonylfluoride group-based warhead were prepared according to the strategy described earlier and outlined in FIG. 20. Using this approach, a series of m5-st4-derived cyclic peptides were prepared, in which a side-chain sulfonylfluoride group was installed at position 13 (i.e., compound m5-s4.1 through m5-s4.3, Table 4). According to a model of the m5-st4/Shh complex, the side-chain of residue in the peptide is proximal to two lysine residues from the Shh protein, potentially enabling the formation of a covalent complex upon reaction of the lysine residue with the sulfonylfluoride moiety. In the three functionalized peptides, the distance of the sulfonylfluoride group from the peptide backbone as well as its orientation in space was varied by using an ornithine (Orn), a 2,3-diaminopropionic acid (Dap), and a D-lysine (DLys), respectively, for providing the side-chain amino group for conjugation of the 4-(fluorosulfonyl)benzoyl group to the peptide. As illustrated in FIG. 20, the three peptides were synthesized by SPPS via the on-resin cyclization method, followed by selective deprotection of the Alloc-protected amino acid and coupling to 4-(fluorosulfonyl)benzoate. After purification, the three peptides were tested for their Shh binding affinity and found to bind Shh with a comparable affinity to m5-st4.

Synthesis of sulfonylfluoride-functionalized peptide. The N-acetylated cyclic peptide corresponding m5-st4.1, m5-st4.2, and m5-st4.3, were assembled via SPPS and Fmoc chemistry using the on-resin cyclization method described in Example 4. N-Fmoc-Orn(Alloc), N-Fmoc-Dap(Alloc), N-Fmoc-(D)-Lys(Alloc) were used as building block for residue 13. After the on-resin cyclization step, the Alloc group was removed with Pd(PPh₃)₄ and PhSiH₃ in DCM and p-(fluorosulfonyl)benzoic acid was coupled to the deprotected sidechain amino group of the resin-bound peptide using PyBOP (3 equiv) and DIPEA (6 equiv of) in DMF. The peptides was cleaved from the resin and isolated according to the standard procedures described earlier.

The terms and expression that are employed herein are used as terms of description and not of limitation, and there is no intention that in the use of such terms and expressions of excluding any equivalents of the features shown and described and portions thereof, but it is recognized that various modifications are possible within the scope of the invention claimed. Thus, it should be understood that although the present invention has been specifically disclosed by various embodiments and optional features, modification and variation of the concepts herein disclosed may be resorted to those skilled in the art, and that such modifications and variations are considered to be within the scope of the invention as defined by the appended claims.

Unless otherwise indicated, the disclosure is not limited to specific molecular structures, substituents, synthetic methods, reaction conditions, or the like, as such may vary. It is to be understood that the embodiments are not limited to particular compositions or biological systems, which can, of course, vary.

A skilled artisan will appreciate that starting materials, biological materials, reagents, synthetic methods, purification methods, analytical methods, assay methods, and biological methods other than those specifically exemplified can be employed in the practice of the invention. All art-known functional equivalents of any such materials and methods are intended to be included in the invention.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

### REFERENCES

Azoulay, S., S. Terry, et al. (2008). J. Pathol. 216(4): 460-470.
Beachy, P. A., S. S. Karhadkar, et al. (2004). Nature 432(7015): 324-331.
Berman, D. M., S. S. Karhadkar, et al. (2003). Nature 425(6960): 846-851.
Bionda, N., A. L. Cryan, et al. (2014). ACS Chem. Biol. 9(9): 2008-2013.
Bishop, B., A. R. Aricescu, et al. (2009). Nat. Struct. Mol. Biol. 16(7): 698-U629.
Bosanac, I., H. R. Maun, et al. (2009). Nat. Struct. Mol. Biol. 16(7): 691-697.
Carpenter, D., D. M. Stone, et al. (1998). Proc. Natl. Acad. Sci. USA 95(23): 13630-13634.
Chames, P., M. Van Regenmortel, et al. (2009). British Journal of Pharmacology 157(2): 220-233.
Chatterjee, J., C. Gilon, et al. (2008). Acc. Chem. Res. 41(10): 1331-1342.
Chen, J. K., J. Taipale, et al. (2002). Proc. Natl. Acad. Sci. USA 99(22): 14071-14076.
Chiang, C., L. T. T. Ying, et al. (1996). Nature 383(6599): 407-413.
Chuang, P. T., T. Kawcak, et al. (2003). Genes Dev. 17(3): 342-347.
Chuang, P. T. and A. P. McMahon (1999). Nature 397(6720): 617-621.
Cooper, M. K., J. A. Porter, et al. (1998). Science 280(5369): 1603-1607.
Denef, N., D. Neubuser, et al. (2000). Cell 102(4): 521-531.
Dierks, C., R. Beigi, et al. (2008). Cancer Cell 14(3): 238-249.
Dierks, C., J. Grbic, et al. (2007). Nat. Med. 13(8): 944-951.
Hegde, G. V., K. J. Peterson, et al. (2008). Mol. Cancer Res. 6(12): 1928-1936.
Ibuki, N., M. Ghaffari, et al. (2013). Int. J. Cancer. 133(8): 1955-1966.
Ingham, P. W. and A. P. McMahon (2001). Gene Dev. 15(23): 3059-3087.
Ingham, P. W. and A. P. McMahon (2001). Genes Dev. 15(23): 3059-3087.
Ingham, P. W. and M. Placzek (2006). Nat. Rev. Genet. 7(11): 841-850.
Jiang, J. and C. C. Hui (2008). Dev. Biol. 15(6): 801-812.
Kasper, M., G. Regl, et al. (2006). Eur. J. Cancer 42(4): 437-445.
Kim, J., M. Kato, et al. (2009). Proc. Natl. Acad. Sci. USA 106(51): 21666-21671.
Lees, C., S. Howie, et al. (2005). Gastroenterology 129(5): 1696-1710.
Liu, C. C. and P. G. Schultz (2010). Annu. Rev. Biochem. 79: 413-444.
Liu, S. L., G. Dontu, et al. (2006). Cancer Res. 66(12): 6063-6071.
Martinez-Chinchilla, P. and N. A. Riobo (2008). Method. Enzymol. 446: 189-204.
Olsen, C. L., P. P. Hsu, et al. (2004). BMC Cancer 4.
Peukert, S. and K. Miller-Moslin (2010). Chemmedchem 5(4): 500-512.
Reya, T., S. J. Morrison, et al. (2001). Nature 414(6859): 105-111.
Robarge, K. D., S. A. Brunton, et al. (2009). Bioorg. Med. Chem. Lett. 19(19): 5576-5581.
Rubin, L. L. and F. J. de Sauvage (2006). Nature Rev. Drug Discov. 5(12): 1026-1033.
Sharpe, H. J., W. Wang, et al. (2015). Nat. Chem. Biol. 11(4): 246-255.
Sinha, S. and J. K. Chen (2006). Nat. Chem. Biol. 2(1): 29-30.
Smith, J. M., F. Vitali, et al. (2011). Angew. Chem. Int. Ed. Engl. 50(22): 5075-5080.
Stanton, B. Z. and L. F. Peng (2010). Mol. Biosyst. 6(1): 44-54.
Stanton, B. Z., L. F. Peng, et al. (2009). Nat. Chem. Biol. 5(3): 154-156.
Tada, M., F. Kanai, et al. (2008). Clin. Cancer Res. 14(12): 3768-3776.
Thayer, S. P., M. P. di Magliano, et al. (2003). Nature 425(6960): 851-856.
Theunissen, J. W. and F. J. de Sauvage (2009). Cancer Res. 69(15): 6007-6010.
Tian, H., C. A. Callahan, et al. (2009). Proc. Natl. Acad. Sci. USA 106(11): 4254-4259.
Tojo, M., H. Kiyosawa, et al. (2002). Brit. J. Dermatol. 146(1): 69-73.
Varjosalo, M. and J. Taipale (2008). Genes & Development 22(18): 2454-2472.
Watkins, D. N., D. M. Berman, et al. (2003). Nature 422(6929): 313-317.
Wen, X., C. K. Lai, et al. (2010). Mol. Cell Biol. 30(8): 1910-1922.
Wong, S. Y. and J. F. Reiter (2008). Curr. Top. Dev. Biol. 85: 225-260.
Yauch, R. L., S. E. Gould, et al. (2008). Nature 455(7211): 406-U461.
Yoon, J. W., Y. Kita, et al. (2002). J. Biol. Chem. 277(7): 5548-5555.
Zhao, C., A. Chen, et al. (2009). Nature 460(7255): 652-652.

1. A cyclic peptide of Formula (I) or Formula (II), or a pharmaceutically acceptable salt thereof: 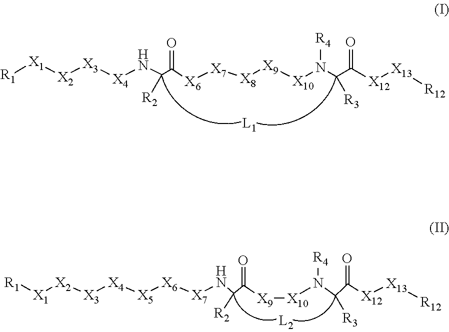
wherein:
X₁ is Thr, Asp, Phe, Glu, Asn, Gln, Ser, Tyr, substituted Phe, substituted Tyr, or substituted Trp;
X₂ is Leu, His, Ser, Val, Trp, Ile, Phe, Tyr, Thr, Nval, Nleu, substituted Phe, substituted Tyr, or substituted Trp;
X₃ is Asp, Ser, Lys, Thr, Val, Asn, Leu, Glu, Ile, Gln, Arg, Orn, Dab, Dap, Nleu, or Nval;
X₄ is Asp, Trp, Gly, Ala, Pro, Tyr, Phe, Glu, Aib, substituted Phe, substituted Tyr, substituted Trp, or an amino acid of formula (IV) 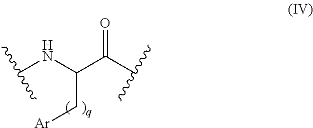
wherein:
q is 0, 1, or 2; and Ar is a C₅-C₁₅ aryl group or a C₅-C₁₅ substituted aryl group;
X₆ is Glu, Ser, Leu, Val, Trp, Gly, Ala, Arg, Tyr, Phe, Ile, Thr, Asp, Lys, Orn, Dab, Dap, substituted Phe, substituted Tyr, substituted Trp, Nval, or Nleu;
X₇ is Glu, Ala, Lys, Gly, Tyr, Phe, Arg, Asp, Orn, Dab, Dap, substituted Phe, substituted Tyr, substituted Trp, Aib, 3,3,3-trifluoro-alanine, alpha,alpha-bis-trifluoromethyl-glycine, or 2-cyclopropylglycine;
X₈ is Met, Nleu, Gly, or Pro;
X₉ is Asp, Glu, Cys, His, or an amino acid residue of formula (III) 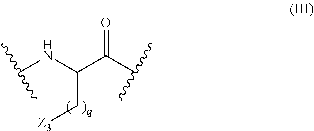
wherein:
q is 0, 1, or 2; and Z₃ is selected from the group consisting of —CONHOH, —N(OH)COCH₃, —CONHOCH₃, —ONHCOCH₃, —P(═O)(OH)₂, —P(═O)H(OH), —SO₃H, —SO₂NH₂, —NHSO₂CH₃, —CONHSO₂CH₃, —NHCONHSO₂CH₃, imidazole, 1,2,3-triazole, 1,2,4-triazole, tetrazole, —CH(OH)CF₃, —C(OH)₂CF₃, thiazolidine-2,4-dione, oxazolidine-2,4-dione, 1,2,4-oxadiazol-5(4H)-one, 1,2,4-thiadiazol-5(4H)-one, 3H-1,2,3,5-oxathiadiazole 2-oxide, 1,2,4-oxadiazole-5(4H)-thione, isoxazol-3-ol, isothiazol-3-ol, pyrrolidine-2,4-dione, furan-2,4(3H,5H)-dione, 3-hydroxycyclopent-2-enone, 3-hydroxycyclobut-3-ene-1,2-dione, and 2,6-difluorophenol;
X₁₀ is Gly, Met, D-Met, Nleu, D-Nleu, Thr, D-Thr, Ser, D-Ser, or —N(R₉)CH₂C(O)—, wherein R₉ is a C₁-C₁₅ aliphatic, C₁-C₁₅ substituted aliphatic, C₅-C₁₅ aryl, C₅-C₁₅ substituted aryl, C₆-C₁₅ alkylaryl, or C₆-C₁₅ substituted alkylaryl group;
X₁₂ is Ser, Thr, Met, Leu, Ile, Val, Nleu, or Nval;
X₁₃ is Asp, Glu, or absent;
X₅ is Met, Gly, or Nleu;
R₁ is hydrogen, an acetyl group, a label molecule, or a R₁₃CO— group, wherein R₁₃ is C₅-C₁₅ alkanoic acid or C₅-C₁₅ alkenoic acid;
R₂ and R₃ are, independently, a hydrogen or an alkyl group comprising between one and three carbon atoms;
R₄ is hydrogen, or a linear or branched alkyl group comprising between one and eight carbon atoms;
R₁₂ is —OH or NH₂;
L₁ is a linker unit, such that the linear dimension between the Cα carbon atoms connected by the linker unit is between about 6 and 15 Angstrom units; and
L₂ is a linker unit, such that the linear dimension between the Cα carbon atoms connected by the linker unit is between about 5 and 12 Angstrom units;
and wherein the cyclic peptide is able to bind at least one homolog of a Hedgehog protein.

2. A cyclic peptide of Formula (I), or a pharmaceutically acceptable salt thereof: 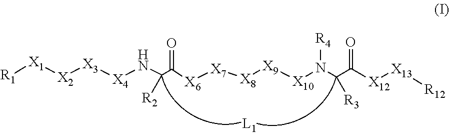
wherein:
X₁ is Thr, Asp, Phe, Glu, Asn, Gln, Ser, or Tyr;
X₂ is Leu, His, Ser, Val, Trp, Ile, Phe, Tyr, or Thr;
X₃ is Asp, Ser, Lys, Thr, Val, Asn, Leu, Glu, Ile, Gln, Gly, Pro, Ala, Trp or Arg;
X₄ is Asp, Trp, Gly, Ala, Pro, Tyr, Phe, or Glu;
X₆ is Glu, Ser, Leu, Val, Trp, Gly, Ala, Arg, Tyr, Phe, Ile, Thr, Asp, or Lys;
X₇ is Glu, Ala, Lys, Gly, Tyr, Phe, Arg, or Asp;
X₈ is Met, Nleu, Gly, or Pro;
X₉ is Asp or Glu;
X₁₀ is Gly, Met, Nleu, Thr, or Ser;
X₁₂ is Ser, Thr, Met, Leu, Ile, or Val;
X₁₃ is Asp, Glu, or absent;
R₁ is hydrogen, an acetyl group, or an acyl group;
R₂ and R₃ are, independently, a hydrogen or an alkyl group comprising between one and three carbon atoms;
R₄ is hydrogen, or a linear or branched alkyl group comprising between one and eight carbon atoms;
R₁₂ is —OH or NH₂; and
L₁ is a linker unit, such that the linear dimension between the Cα carbon atoms connected by the linker unit is between about 6 and 15 Angstrom units.

3. The cyclic peptide of claim 2, wherein X₁ is Thr; X₂ is Leu, His, Ser, Val, or Trp; X₃ is Asp, or Ser; X₄ is Asp, Trp, or Gly; X₆ is Glu, Ser, Leu, Val, Trp, Gly, Ala, Arg, Tyr, or Phe; X₇ is Glu, Ala, Lys, or Gly; X₈ is Met; X₉ is Asp; X₁₀ is Gly, or Met; X₁₂ is Ser, or Thr; X₁₃ is Asp.

4. A cyclic peptide of Formula (II), or a pharmaceutically acceptable salt thereof: 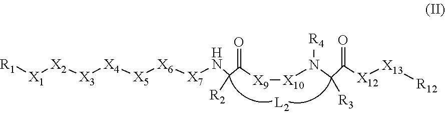
wherein:
X₁ is Thr, Asp, Phe, Glu, Asn, Gln, Ser, or Tyr;
X₂ is Leu, His, Ser, Val, Trp, Ile, Phe, Tyr, or Thr;
X₃ is Asp, Ser, Lys, Thr, Val, Asn, Leu, Glu, Ile, Gln, or Arg;
X₄ is Asp, Trp, Gly, Ala, Pro, Tyr, Phe, or Glu;
X₅ is Met or Gly;
X₆ is Glu, Ser, Leu, Val, Trp, Gly, Ala, Arg, Tyr, Phe, Ile, Thr, Asp, or Lys;
X₇ is Glu, Ala, Lys, Gly, Tyr, Phe, Arg, or Asp;
X₉ is Asp or Glu;
X₁₀ is Gly, Met, Nleu, Thr, or Ser;
X₁₂ is Ser, Thr, Met, Leu, Ile, or Val;
X₁₃ is Asp, Glu, or absent;
R₁ is hydrogen, an acetyl group, or an acyl group;
R₂ and R₃ are, independently, a hydrogen or an alkyl group comprising between one and three carbon atoms;
R₄ is hydrogen or an alkyl group comprising between one and eight carbon atoms;
R₁₂ is —OH or NH₂; and
L₂ is a linker unit, such that the linear dimension between the Cα carbon atoms connected by the linker unit is between about 5 and 12 Angstrom units.

5. The cyclic peptide of claim 4, wherein X₁ is Thr; X₂ is Leu; X₃ is Asp; X₄ is Asp; X₅ is Met; X₆ is Glu; X₇ is Glu; X₉ is Asp; X₁₀ is Gly; X₁₂ is Ser, Leu, or Val; X₁₃ is Asp.

6. The cyclic peptide of claim 1, wherein L₁ is —R₉—S—R₁₀— or —R₁₀—S—R₉—, wherein R₉ is an alkylaryl or a substituted alkylaryl group comprising between 5 and 20 carbon atoms, and R₁₀ is an alkyl or a substituted alkyl group comprising between 1 and 10 carbon atoms.

7. The cyclic peptide of claim 6, wherein L₁ is 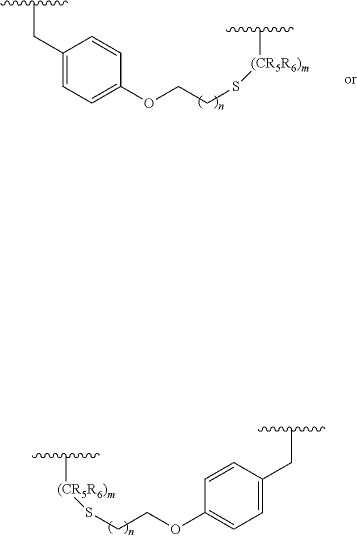
wherein n is an integer number comprised between 1 and 6; m is an integer number comprised between 1 and 3; and R₅ and R₆ are, independently, a hydrogen atom or a methyl group.

8. The cyclic peptide of claim 1, wherein L₁ is —(CH₂)ₙ—Z₁—(CH₂)ₚ—, wherein Z₁ is —S—, —S—S—, —NHCO—, —CONH—, —CH═CH—, —(CH₂)₂—, —CH—CH—, or a triazole group, and n and p are, independently, an integer number between 1 and 6.

9. The cyclic peptide of claim 1, wherein L₁ is —C(R₅R₆)ₘ—S—Z₂—S—C(R₇R₈)ₒ, wherein Z₂ is selected from the group consisting of a C₁-C₁₀ alkyl, C₁-C₁₀ substituted alkyl, C₂-C₁₀ alkenyl, C₂-C₁₀ substituted alkenyl, C₆-C₁₂ aryl, C₆-C₁₂ substituted aryl, C₅-C₁₄ alkylaryl, C₅-C₁₄ substituted alkylaryl group, —CH₂COCH₂—, and —CH₂COCOCH₂—; m and o are, independently, an integer number comprised between 1 and 3; R₅, R₆, R₇, and R₈ are, independently, a hydrogen atom or a methyl group.

10. The cyclic peptide of claim 1, wherein L₂ is —R₉—S—R₁₀— or —R₁₀—S—R₉—, wherein R₉ is an alkylaryl or a substituted alkylaryl group comprising between 5 and 20 carbon atoms, and R₁₀ is an alkyl or a substituted alkyl group comprising between 1 and 10 carbon atoms.

11. The cyclic peptide of claim 10, wherein L₂ is 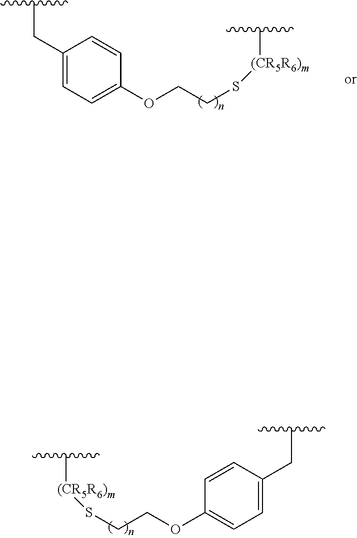
wherein n is an integer number comprised between 1 and 6; m is an integer number comprised between 1 and 3; and R₅ and R₆ are, independently, a hydrogen atom or a methyl group.

12. The cyclic peptide of claim 1, wherein L₂ is —(CH₂)ₙ—Z₁—(CH₂)ₚ—, wherein Z₁ is —S—, —S—S—, —NHCO—, —CONH—, —CH═CH—, —(CH₂)₂—, —CH—CH—, or a triazole group, and n and p are, independently, an integer number between 1 and 6.

13. The cyclic peptide of claim 1, wherein L₂ is —C(R₅R₆)ₘ—S—Z₂—S—C(R₇R₈)ₒ, wherein Z₂ is selected from the group consisting of a C₁-C₁₀ alkyl, C₁-C₁₀ substituted alkyl, C₂-C₁₀ alkenyl, C₂-C₁₀ substituted alkenyl, C₆-C₁₂ aryl, C₆-C₁₂ substituted aryl, C₈-C₁₄ alkylaryl, C₈-C₁₄ substituted alkylaryl group, —CH₂COCH₂—, and —CH₂COCOCH₂—; m and o are, independently, an integer number comprised between 1 and 3; R₅, R₆, R₇, and R₈ are, independently, a hydrogen atom or a methyl group.

14. The cyclic peptide of claim 1, wherein at least one among residues X₁, X₂, X₃, X₄, X₅, X₆, and X₇ is an alpha,alpha-disubstituted amino acid carrying an alpha-methyl group.

15. The cyclic peptide of claim 1, wherein at least one of residue X₁, X₂, X₁₀, X₁₁, X₁₂, and X₁₃ is an N-methylated amino acid.

16. The cyclic peptide of claim 1, wherein the peptide comprises a warhead group.

17. The cyclic peptide of claim 16, wherein the warhead group is —SO₂F, —Ar₂—SO₂F, —(CH₂)ₙSO₂F, —Ar₂—NCS, —NHSO₂CH═CH₂, or —(CH₂)ᵣCH₂Br, wherein Ar₂ is a C₅-C₁₀ aryl or a C₅-C₁₀ substituted aryl group; n is an integral number between 1 and 6; r is an integral number between 1 and 10.

18. The cyclic peptide of claim 1, wherein L₁ is —CH₂SSCH₂—, —CH₂SCH₂SCH₂—, —CH₂S(CH₂)₂SCH₂—, —CH₂S(CH₂)₃SCH₂—, —CH₂S(CH₂)₄SCH₂—, —CH₂S(CH₂)₅SCH₂—, —CH₂SCH₂CH═CHCH₂SCH₂CH₂—,

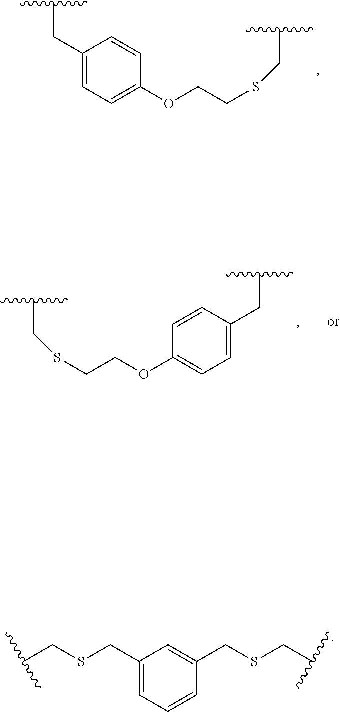 [SMILES: *CSCCOc1ccc(C*)cc1 ; *CSCCOc1ccc(C*)cc1 ; *CSCc1cccc(CSC*)c1]

19. The cyclic peptide of claim 1, wherein the peptide is one selected from a peptide set forth in Tables 1, 2, 3 or 4.

20. The cyclic peptide of claim 1, wherein the peptide comprises a label molecule selected from an affinity label molecule, a photoaffinity label, a dye, a chromophore, a fluorescent molecule, a phosphorescent molecule, a chemiluminescent molecule, an energy transfer agent, a photocrosslinker molecule, a redox-active molecule, an isotopic label molecule, a spin label molecule, a radioactive moiety, a contrast agent molecule, a MRI contrast agent, an isotopically labeled molecule, a PET agent, an electron dense group, a magnetic group, a cofactor, a biotin, a biotin analogue or a combination thereof.


SEQUENCE LISTING (72 sequences)

SEQ ID NO: 1 - protein, 13 aa, Artificial Sequence
  Engineered sequence
TLDDMEEMDGLSD

SEQ ID NO: 2 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEEMDGSD

SEQ ID NO: 3 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEEMDGMD

SEQ ID NO: 4 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDWEEMDMTD

SEQ ID NO: 5 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLSWEAMDMTD

SEQ ID NO: 6 - protein, 27 aa, Artificial Sequence
  Engineered sequence
MDYKDDDDKGSGSTLDDMEEMDGLSDT

SEQ ID NO: 7 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEEMDGSD

SEQ ID NO: 8 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLWDEEMDGSD

SEQ ID NO: 9 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLGDEEMDGSD

SEQ ID NO: 10 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLADEEMDGSD

SEQ ID NO: 11 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLPDEEMDGSD

SEQ ID NO: 12 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDSEMDGSD

SEQ ID NO: 13 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDLEMDGSD

SEQ ID NO: 14 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDVEMDGSD

SEQ ID NO: 15 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDWEMDGSD

SEQ ID NO: 16 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEKMDGSD

SEQ ID NO: 17 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEAMDGSD

SEQ ID NO: 18 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEGMDGSD

SEQ ID NO: 19 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEYMDGSD

SEQ ID NO: 20 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEEMDTSD

SEQ ID NO: 21 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEEMDMSD

SEQ ID NO: 22 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEEMDGTD

SEQ ID NO: 23 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEEMDGLD

SEQ ID NO: 24 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEEMDGMD

SEQ ID NO: 25 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDVEMDGSD

SEQ ID NO: 26 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDGEMDGLD

SEQ ID NO: 27 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDAEMDGMD

SEQ ID NO: 28 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDVEMDGLD

SEQ ID NO: 29 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEKMDMMD

SEQ ID NO: 30 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDDEYMDMMD

SEQ ID NO: 31 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDWEEMDTLD

SEQ ID NO: 32 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDGEEMDMMD

SEQ ID NO: 33 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDWEEMDTMD

SEQ ID NO: 34 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDWEEMDMTD

SEQ ID NO: 35 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLSWEEMDMTD

SEQ ID NO: 36 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLDWEAMDMTD

SEQ ID NO: 37 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLSWEAMDMTD

SEQ ID NO: 38 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLSWLAMDMTD

SEQ ID NO: 39 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLSWFAMDMTD

SEQ ID NO: 40 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLSWYAMDMTD

SEQ ID NO: 41 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TLSWRAMDMTD

SEQ ID NO: 42 - protein, 11 aa, Artificial Sequence
  Engineered sequence
  MISC_FEATURE (4)..(5): Linking point between amino acid and linker
  MISC_FEATURE (9)..(10): Linking point between amino acid and linker
THSWEAMDMTD

SEQ ID NO: 43 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TSSWEAMDMTD

SEQ ID NO: 44 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TVSWEAMDMTD

SEQ ID NO: 45 - protein, 11 aa, Artificial Sequence
  Engineered sequence
  MISC_FEATURE (4)..(5): Linking point between amino acid and linker
  MISC_FEATURE (9)..(10): Linking point between amino acid and linker
TWSWEAMDMTD

SEQ ID NO: 46 - protein, 11 aa, Artificial Sequence
  Engineered sequence
  MISC_FEATURE (4)..(5): Linking point between amino acid and linker
  MISC_FEATURE (9)..(10): Linking point between amino acid and linker
DLSWEAMDMTD

SEQ ID NO: 47 - protein, 11 aa, Artificial Sequence
  Engineered sequence
  MISC_FEATURE (4)..(5): Linking point between amino acid and linker
  MISC_FEATURE (9)..(10): Linking point between amino acid and linker
FLSWEAMDMTD

SEQ ID NO: 48 - protein, 11 aa, Artificial Sequence
  Engineered sequence
  MISC_FEATURE (4)..(5): Linking point between amino acid and linker
  MISC_FEATURE (9)..(10): Linking point between amino acid and linker
ELSWEAMDMTD

SEQ ID NO: 49 - protein, 11 aa, Artificial Sequence
  Engineered sequence
  MISC_FEATURE (4)..(5): Linking point between amino acid and linker
  MISC_FEATURE (9)..(10): Linking point between amino acid and linker
NLSWEAMDMTD

SEQ ID NO: 50 - protein, 11 aa, Artificial Sequence
  Engineered sequence
  MISC_FEATURE (4)..(5): Linking point between amino acid and linker
  MISC_FEATURE (9)..(10): Linking point between amino acid and linker
QLSWEAMDMTD

SEQ ID NO: 51 - protein, 13 aa, Artificial Sequence
  Engineered sequence
MDYKDDDDKGSGS

SEQ ID NO: 52 - protein, 11 aa, Artificial Sequence
  Engineered sequence
  MISC_FEATURE (4)..(5): Linking point between amino acid and linker
  MISC_FEATURE (6)..(6): X= 2-aminoisobutyric acid (Aib)
  MISC_FEATURE (9)..(10): Linking point between amino acid and linker
TLSWEXMDMTD

SEQ ID NO: 53 - protein, 11 aa, Artificial Sequence
  Engineered sequence
  MISC_FEATURE (4)..(5): Linking point between amino acid and linker
  MISC_FEATURE (9)..(9): X= norleucine (Nleu)
  MISC_FEATURE (9)..(10): Linking point between amino acid and linker
TLSWEAMDXTD

SEQ ID NO: 54 - protein, 11 aa, Artificial Sequence
  Engineered sequence
  MISC_FEATURE (4)..(5): Linking point between amino acid and linker
  MISC_FEATURE (9)..(10): Linking point between amino acid and linker
  MISC_FEATURE (9)..(9): X= N-(n-butyl)-glycine
TLSWEAMDXTD

SEQ ID NO: 55 - protein, 11 aa, Artificial Sequence
  Engineered sequence
  MISC_FEATURE (4)..(5): Linking point between amino acid and linker
  MISC_FEATURE (9)..(10): Linking point between amino acid and linker; D-configured amino acid residue.
TLSWEAMDMTD

SEQ ID NO: 56 - protein, 11 aa, Artificial Sequence
  Engineered sequence
  MISC_FEATURE (7)..(8): Linking point between amino acid and linker
  MISC_FEATURE (9)..(10): Linking point between amino acid and linker
TLDDMEEDGSD

SEQ ID NO: 57 - protein, 11 aa, Artificial Sequence
  Engineered sequence
  MISC_FEATURE (7)..(8): Linking point between amino acid and linker
  MISC_FEATURE (9)..(10): Linking point between amino acid and linker
TVSDGEEDGLD

SEQ ID NO: 58 - protein, 11 aa, Artificial Sequence
  Engineered sequence
  MISC_FEATURE (7)..(8): Linking point between amino acid and linker
  MISC_FEATURE (9)..(10): Linking point between amino acid and linker
TRGDGEEDGVD

SEQ ID NO: 59 - protein, 11 aa, Artificial Sequence
  Engineered sequence
  MISC_FEATURE (7)..(8): Linking point between amino acid and linker
  MISC_FEATURE (9)..(10): Linking point between amino acid and linker
TVSDGEEDGLD

SEQ ID NO: 60 - protein, 11 aa, Artificial Sequence
  Engineered sequence
  MISC_FEATURE (4)..(5): Linking point between amino acid and linker
  MISC_FEATURE (9)..(10): Linking point between amino acid and linker
  MISC_FEATURE (11)..(11): X = Orn(Pfsb) = N -(4-(fluorosulfonyl)benzoyl)-L-ornithine
TLSWEAMDMTX

SEQ ID NO: 61 - protein, 11 aa, Artificial Sequence
  Engineered sequence
  MISC_FEATURE (4)..(5): Linking point between amino acid and linker
  MISC_FEATURE (9)..(10): Linking point between amino acid and linker
  MISC_FEATURE (11)..(11): X = Dap(Pfsb) = (S)-2-amino-3-(4-(fluorosulfonyl)benzamido)propanoic acid
TLSWEAMDMTX

SEQ ID NO: 62 - protein, 11 aa, Artificial Sequence
  Engineered sequence
  MISC_FEATURE (4)..(5): Linking point between amino acid and linker
  MISC_FEATURE (9)..(10): Linking point between amino acid and linker
  MISC_FEATURE (11)..(11): X = DLys(Pfsb) = N -(4-(fluorosulfonyl)benzoyl)-D-lysine
TLSWEAMDMTX

SEQ ID NO: 63 - protein, 26 aa, Artificial Sequence
  Engineered sequence
MDYKDDDDKGSGSTLDDMEEMDGLSD

SEQ ID NO: 64 - protein, 25 aa, Artificial Sequence
  Engineered sequence
MDYKDDDDKGSGSTLDDEEMDGCSD

SEQ ID NO: 65 - protein, 25 aa, Artificial Sequence
  Engineered sequence
MDYKDDDDKGSGSTLDDCEEMDGSD

SEQ ID NO: 66 - protein, 26 aa, Artificial Sequence
  Engineered sequence
MDYKDDDDKGSGSTLDDMEEMDGLSD

SEQ ID NO: 67 - protein, 13 aa, Artificial Sequence
  Engineered sequence
  MISC_FEATURE (5)..(5): X=Tyrosine analog (Y*)
  MISC_FEATURE (11)..(11): Linking point between amino acid and linker
TLDDXEEMDGCSD

SEQ ID NO: 68 - protein, 5 aa, Artificial Sequence
  Engineered sequence
EAMDM

SEQ ID NO: 69 - protein, 13 aa, Artificial Sequence
  Engineered sequence
  MISC_FEATURE (5)..(5): X=Tyrosine analog (Y*)
  MISC_FEATURE (11)..(11): Linking point between amino acid and linker
TLSWXEAMDMCTD

SEQ ID NO: 70 - protein, 13 aa, Artificial Sequence
  Engineered sequence
TLSWCEAMDMCTD

SEQ ID NO: 71 - protein, 13 aa, Artificial Sequence
  Engineered sequence
  MISC_FEATURE (13)..(13): X = Diaminopimelic acid (Dap)
TLSWCEAMDMCTX

SEQ ID NO: 72 - protein, 4 aa, Artificial Sequence
  Engineered sequence
GSGS